(19) United States
(12) Patent Application Publication
(10) Pub. No.: US 2023/0243827 A1
(43) Pub. Date: Aug. 3, 2023

(54) CORONAVIRUS DIAGNOSTIC COMPOSITIONS, METHODS, AND USES THEREOF

(71) Applicant: Sichuan Clover Biopharmaceuticals, Inc., Chengdu Sichuan (CN)

(72) Inventors: Peng LIANG, Chengdu Sichuan (CN); Joshua LIANG, Chengdu Sichuan (CN)

(73) Assignee: Sichuan Clover Biopharmaceuticals, Inc., Chengdu Sichuan (CN)

(21) Appl. No.: 18/009,701

(22) PCT Filed: Jun. 10, 2021
(86) PCT No.: PCT/CN2021/099293

§ 371 Date: Dec. 9, 2022

(30) Foreign Application Priority Data
Jun. 10, 2020 (WO) PCT/CN2020/095332
Apr. 13, 2021 (WO) PCT/CN2021/087051

(51) Int. Cl. G01N 33/569; G01N 33/543

(52) U.S. Cl. CPC G01N 33/56983; G01N 33/54388; G01N 2333/165; G01N 2469/20

(57) ABSTRACT

The present disclosure discloses recombinant peptides and proteins comprising coronavirus viral antigens and immunogens, e.g., coronavirus S protein peptides, useful for analyzing an analyte such as neutralizing antibodies. In some aspects, the recombinant peptides and proteins comprise a secreted fusion protein comprising a soluble coronavirus viral antigen joined by in-frame fusion to a C-terminal portion of a collagen which is capable of self-trimerization to form a disulfide bond-linked trimeric fusion protein. Diagnostic methods and related kits are also disclosed.

### CROSS-REFERENCE TO RELATED APPLICATIONS

This application claims priority to and the benefit of International Patent Application Nos. PCT/CN2020/095332, filed Jun. 10, 2020, and PCT/CN2021/087051, filed Apr. 13, 2021, the disclosures of which applications are incorporated herein by reference in their entireties for all purposes.

### SUBMISSION OF SEQUENCE LISTING AS ASCII TEXT FILE

The content of the following submission on ASCII text file is incorporated herein by reference in its entirety: a computer readable form (CRF) of the Sequence Listing (file name: 165762000542SEQLIST.TXT, date recorded: Jun. 9, 2021, size: 575 KB).

### FIELD

The present disclosure relates in some aspects to recombinant peptides and proteins comprising coronavirus viral antigens and immunogens, e.g., coronavirus S protein peptides, for detecting and/or analyzing a coronavirus infection, e.g., for the purpose of diagnosing the coronavirus infection.

### BACKGROUND

Coronaviruses are enveloped, positive-sense single-stranded RNA viruses. They have the largest genomes (26-32 kb) among known RNA viruses, and are phylogenetically divided into four genera (a, R, y, 8), with betacoronaviruses further subdivided into four lineages (A, B, C, D). Coronaviruses infect a wide range of avian and mammalian species, including humans. Human coronaviruses may circulate annually in humans and generally cause mild respiratory diseases, although severity can be greater in infants, elderly, and the immunocompromised. In contrast, certain other coronaviruses, including the Middle East respiratory syndrome coronavirus (MERS-CoV), the severe acute respiratory syndrome coronavirus (SARS-CoV), and the most recent 2019 new coronavirus (2019-nCoV), also known as SARS-CoV-2, are highly pathogenic. The high pathogenicity and airborne transmissibility of these coronaviruses have raised concern about the potential for another coronavirus pandemic. There is an urgent need for effective tests for diagnosing coronavirus infection. Provided are methods, uses and articles of manufacture that meet such and other needs.

### SUMMARY

In some aspects, provided herein are methods for analyzing a sample, comprising: contacting a sample with a protein (e.g., an S-Trimer, NTD/RBD-Trimer, RBD-Trimer, S1-Trimer, or S2-Trimer disclosed herein) comprising an S protein peptide or fragment or epitope thereof of a coronavirus, and detecting a binding between the protein and an analyte capable of specific binding to the S protein peptide or fragment or epitope thereof of the coronavirus. In some embodiments, the analyte is an antibody, a receptor, or a cell recognizing the S protein peptide or fragment or epitope thereof. In some embodiments, the binding indicates the presence of the analyte in the sample, and/or an infection by the coronavirus in a subject from which the sample is derived.

In some aspects, the methods herein provide sensitive detection of an analyte capable of specific binding to the S protein peptide or fragment or epitope thereof, either during viral infections and/or after vaccination with a protein or peptide disclosed herein. In any of the preceding embodiments, the analyte can be an IgG antibody, an IgM antibody, or an IgE antibody, e.g., one that is specific to an S protein peptide or fragment or epitope thereof. In any of the preceding embodiments, the analyte can be a neutralizing antibody against the coronavirus, such as SARS-CoV-2. In any of the preceding embodiments, the method can be an ELISA or lateral flow assay.

In some aspects, provided herein are kits comprising the protein provided herein and a substrate, pad, or vial containing or immobilizing the protein, optionally wherein the kit is an ELISA or lateral flow assay kit.

In some embodiments of the method disclosed herein, the protein is immobilized within a test zone of a chromatographic strip on a test strip.

In any of the preceding embodiments, the chromatographic strip can further comprise a control zone, and wherein a control capture agent is immobilized within the control zone.

In any of the preceding embodiments, the test strip can further comprise a sample binding zone comprising a binding pad, and one end of the binding pad is in capillary communication with one end of the chromatographic strip.

In any of the preceding embodiments, the test strip can further comprise a sample addition zone comprising a sample pad, wherein the sample pad can be in capillary communication with the binding pad or the chromatographic strip.

In any of the preceding embodiments, the analyte can be a neutralizing antibody against the surface antigen of the coronavirus.

In any of the preceding embodiments, the analyte can be a broad neutralizing antibody against the surface antigen of the coronavirus.

In any of the preceding embodiments, the analyte can be an IgG antibody, e.g, one that is specific to an S protein peptide or fragment or epitope thereof.

In any of the preceding embodiments, the analyte can be an IgM antibody, e.g, one that is specific to an S protein peptide or fragment or epitope thereof.

In any of the preceding embodiments, the analyte can be an IgE antibody, e.g, one that is specific to an S protein peptide or fragment or epitope thereof.

In any of the preceding embodiments, the analyte can be an IgA antibody, e.g, one that is specific to an S protein peptide or fragment or epitope thereof.

In any of the preceding embodiments, the analyte can be an IgD antibody, e.g, one that is specific to an S protein peptide or fragment or epitope thereof.

In any of the preceding embodiments, the analyte can be a human antibody, e.g, one that is specific to an S protein peptide or fragment or epitope thereof.

In any of the preceding embodiments, the sample can be derived from a subject infected with the coronavirus.

In any of the preceding embodiments, the sample can be serum from a subject infected with the coronavirus and has recovered.

In any of the preceding embodiments, the sample can further comprise a receptor for the surface antigen of the coronavirus.

In any of the preceding embodiments, the sample can comprise a neutralizing antibody that blocks interaction between the receptor and the surface antigen of the coronavirus.

In some embodiments, disclosed herein is a protein comprising a plurality of recombinant polypeptides, each recombinant polypeptide comprising a surface antigen of a coronavirus linked to a C-terminal propeptide of collagen, wherein the C-terminal propeptides of the recombinant polypeptides form inter-polypeptide disulfide bonds.

In some embodiments, the coronavirus is a Severe Acute Respiratory Syndrome (SARS)-coronavirus (SARS-CoV), a SARS-coronavirus 2 (SARS-CoV-2), a SARS-like coronavirus, a Middle East Respiratory Syndrome (MERS)-coronavirus (MERS-CoV), a MERS-like coronavirus, NL63-CoV, 229E-CoV, OC43-CoV, HKU1-CoV, WIV1-CoV, MHV, HKU9-CoV, PEDV-CoV, or SDCV.

In any of the preceding embodiments, the surface antigen can comprise a coronavirus spike (S) protein or a fragment or epitope thereof, wherein the epitope is optionally a linear epitope or a conformational epitope, and wherein the protein comprises three recombinant polypeptides.

In some embodiments, the coronavirus S protein fusion peptides comprise an ecto-domain (e.g., without transmembrane and cytoplasmic domains) of an S protein or its fragments from a coronavirus, such as SARS-CoV-2, which is fused in-frame to a C-propeptide of a collagen that is capable of forming disulfide bond-linked homo-trimer. The resulting recombinant protein, such as an S-trimer, can be expressed and purified from transfected cells, and are expected to be in native-like conformation in trimeric form. This solves the problems of mis-folding of a viral antigen often encountered when it is expressed as a recombinant peptide or protein in soluble forms without the transmembrane and/or cytoplasmic domains. Such mis-folded viral antigens do not faithfully preserve the native viral antigen conformation, and often fail to be recognized by neutralizing antibodies elicited by the virus.

In any of the preceding embodiments, the surface antigen can comprise a signal peptide, an S1 subunit peptide, an S2 subunit peptide, or any combination thereof.

In any of the preceding embodiments, the surface antigen can comprise a signal peptide, a receptor binding domain (RBD) peptide, a receptor binding motif (RBM) peptide, a fusion peptide (FP), a heptad repeat 1 (HR1) peptide, or a heptad repeat 2 (HR2) peptide, or any combination thereof.

In any of the preceding embodiments, the surface antigen can comprises a receptor binding domain (RBD) of the S protein.

In any of the preceding embodiments, the surface antigen can comprise an S1 subunit and an S2 subunit of the S protein.

In any of the preceding embodiments, the surface antigen can be free of a transmembrane (TM) domain peptide and/or a cytoplasm (CP) domain peptide.

In any of the preceding embodiments, the surface antigen can comprise a protease cleavage site, wherein the protease is optionally furin, trypsin, factor Xa, or cathepsin L.

In any of the preceding embodiments, the surface antigen can be free of a protease cleavage site, wherein the protease is optionally furin, trypsin, factor Xa, or cathepsin L, or can contain a mutated protease cleavage site that is not cleavable by the protease.

In any of the preceding embodiments, the surface antigen can be soluble or do not directly bind to a lipid bilayer, e.g., a membrane or viral envelope.

In any of the preceding embodiments, the surface antigens can be the same or different among the recombinant polypeptides of the protein.

In any of the preceding embodiments, the surface antigen can be directly fused to the C-terminal propeptide, or can be linked to the C-terminal propeptide via a linker, such as a linker comprising glycine-X-Y repeats, wherein X and Y and independently any amino acid and optionally proline or hydroxyproline.

In any of the preceding embodiments, the protein can be soluble or do not directly bind to a lipid bilayer, e.g., a membrane or viral envelope.

In any of the preceding embodiments, the protein can bind to a cell surface receptor of a subject, optionally wherein the subject is a mammal such as a primate, e.g., human.

In any of the preceding embodiments, the cell surface receptor can be angiotensin converting enzyme 2 (ACE2), dipeptidyl peptidase 4 (DPP4), dendritic cell-specific intercellular adhesion molecule-3-grabbing non integrin (DC-SIGN), or liver/lymph node-SIGN (L-SIGN).

In any of the preceding embodiments, the C-terminal propeptide can be of human collagen.

In any of the preceding embodiments, the C-terminal propeptide can comprise a C-terminal polypeptide of proα1(I), proα1(II), proα1(III), proα1(V), proα1(XI), proα2(I), proα2(V), proα2(XI), or proα3(XI), or a fragment thereof.

In any of the preceding embodiments, the C-terminal propeptides can be the same or different among the recombinant polypeptides.

In any of the preceding embodiments, the C-terminal propeptide can comprise any of SEQ ID NOs: 67-80 or an amino acid sequence at least 90% identical thereto capable of forming inter-polypeptide disulfide bonds and trimerizing the recombinant polypeptides.

In any of the preceding embodiments, the C-terminal propeptide can comprise a sequence comprising glycine-X-Y repeats (e.g., linked to the N-terminus of any of SEQ ID NOs: 67-80), wherein X and Y and independently any amino acid and optionally proline or hydroxyproline, or an amino acid sequence at least 90% identical thereto capable of forming inter-polypeptide disulfide bonds and trimerizing the recombinant polypeptides.

In any of the preceding embodiments, the surface antigen in each recombinant polypeptide can be in a prefusion conformation.

In any of the preceding embodiments, the surface antigen in each recombinant polypeptide can be in a postfusion conformation.

In any of the preceding embodiments, the surface antigen in each recombinant polypeptide can comprise any of SEQ ID NOs: 27-66 or an amino acid sequence at least 80% identical thereto.

In any of the preceding embodiments, the recombinant polypeptide can comprise any of SEQ ID NOs: 1-26 or an amino acid sequence at least 80% identical thereto.

### BRIEF DESCRIPTION OF THE DRAWINGS

FIG. 1 shows structural features of an exemplary S-Trimer. (A) Schematic illustration of the structural domains of S-Trimer and (B) its trimeric and covalently-linked three-dimensional conformation.

FIG. 2 shows results of an exemplary S-Trimer antigen-based SARS-CoV-2 antibody test in ELISA format.

FIG. 3 is adapted from Posthuma-Trumpie et al., Anal Bioanal Chem (2009) 393:569-582 and shows an exemplary lateral flow immunoassay (LFIA) in sandwich format. Nanoparticle labelled analyte-binding agent 1 is dried at the conjugate release pad. Analyte-binding agent 2 may be sprayed at the test line (T). A control is sprayed at the control line (C). Sample flows from the sample pad to the conjugate pad and into the membrane. Strips are mounted in a device for protection and easier handling. Either analyte-binding agent 1 or analyte-binding agent 2 may be an S-Trimer that binds to S-reactive antibodies in COVID-19 patient sera.

FIG. 4 is adapted from Posthuma-Trumpie et al., Anal Bioanal Chem (2009) 393:569-582 and shows an exemplary lateral flow (immuno)assay in tube format where the conjugate is dehydrated in a test tube. Tube and strip are stored in a sealed aluminum pouch and a desiccant. To run the test, sample (and buffer) are pipetted into the test tube, conjugate is dissolved and the strip is inserted. Response at the test line (T) is dependent on the analyte concentration; response at the control line (C) indicates a proper flow through the membrane.

FIG. 5 shows results of an exemplary S-Trimer antigen-based SARS-CoV-2 antibody test for IgM and IgG.

FIG. 6 shows results of an exemplary S-Trimer antigen-based SARS-CoV-2 antibody test for IgG and neutralizing antibodies.

FIG. 7 shows lateral flow assay results of serially diluted samples of a convalescent serum using either an S-Trimer (FIG. 7, upper panel) or an S1-Trimer (FIG. 7, lower panel) as the antigen.

FIG. 8 shows lateral flow assay results of multiple samples of convalescent sera using either a prototypic SARS-CoV-2 S-Trimer (FIG. 8, upper panel) or a B.1.351 South African variant S-Trimer (FIG. 8, lower panel) as the antigen.

### DETAILED DESCRIPTION

Point-of-care assays are generally designed to detect an analyte based on a structural feature of that analyte. An example of such an assay is a lateral flow immunoassay. Lateral flow immunoassays are widely used as point-of-care tests across multiple industry sectors, including healthcare diagnostics, disease diagnostics, environmental testing, animal health testing, and food and feed testing. Most lateral flow assays use either a sandwich format or a competitive format (Dzantiev et al., TrAC Trends in Analytical Chemistry, 55, 2014; Sajid et al., Journal of Saudi Chemical Society, 19, 2015). In an exemplary sandwich format, primary antibodies specific to a target analyte are immobilized at a test line and labeled antibodies specific to the target analyte are loaded in a section of the test strip upstream of the test line. When sample containing the analyte is applied to the test strip, the analyte is captured by the labeled antibodies and flows towards the test line. The immobilized antibodies at the test line then capture the analyte complexed with the labeled antibody, thereby forming a detectable sandwich with the analyte. The test strip may also contain a control line with an immobilized secondary antibody, wherein the labeled antibodies that pass the test line are captured at the control line to ensure proper operation of the test strip. The intensity of color at test line corresponds to the amount of target analyte and can be measured with either an optical strip reader or visual inspection. Competitive formats are often used to examine low molecular weight compounds which are too small to bind to two antibodies simultaneously, have two general layouts. In the first layout, the test strip has a test line containing an immobilized analyte (the same as being detected), a control line containing an immobilized secondary antibody, and a mobile labeled antibody specific to the analyte loaded in the test strip upstream of the test line. When a sample containing the analyte is applied to the test strip, the mobile labeled antibodies form complexes with the analyte. As the complexes travel down the test strip, the analyte is not bound at the test line and instead is bound at the control line by the immobilized secondary antibodies. When the analyte is not present, the mobile labeled antibodies bind to the immobilized analyte at the test line. In a second layout, the test strip has a test line containing an immobilized antibody specific to the analyte, and a mobile labeled analyte (the same as being detected) loaded in the test strip upstream of the test line. When a sample containing the analyte is applied to the test strip, the mobile labeled analyte competes with the analyte for binding with the immobilized antibodies in the test line and thus less mobile labeled analyte is bound at the test line. Li et al., Analytical Chemistry, 83, 2011.

In the present disclosure, instead of antibodies, coronavirus S protein fusion peptides (e.g., S-Trimer, NTD/RBD-Trimer, S1-Trimer, S2-Trimer, RBD-Trimer, etc.) are used, e.g., in order to detect analytes, such as antigen specific antibodies that recognize the S protein fusion peptides and/or neutralizing antibodies against the viruses (e.g., antibodies that block virus interaction with its cellular receptor(s)).

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference. The section headings used herein are for organizational purposes only and are not to be construed as limiting the subject matter described.

### I. Viral Antigens and Immunogens

The proteins provided herein comprise coronavirus viral antigens and immunogens. The coronavirus viral antigens and immunogens contemplated herein are capable of promoting or stimulating a cell-mediated response and/or a humoral response. In some embodiments, the response, e.g., cell-mediated or humoral response, comprises the production of antibodies, e.g., neutralizing antibodies. In some embodiments, the coronavirus viral antigen or immunogen is an coronavirus S protein peptide.

Coronavirus is a family of positive-sense, single-stranded RNA viruses that are known to cause severe respiratory illness. Viruses currently known to infect human from the coronavirus family are from the alphacoronavirus and betacoronavirus genera. Additionally, it is believed that the gammacoronavirus and deltacoronavirus genera may infect humans in the future. Non-limiting examples of betacoronaviruses include Middle East respiratory syndrome coronavirus (MERS-CoV), Severe Acute Respiratory Syndrome coronavirus (SARS-CoV), Human coronavirus HKU1 (HKU1-CoV), Human coronavirus OC43 (OC43-CoV), Murine Hepatitis Virus (MHV-CoV), Bat SARS-like coronavirus WIV1 (WIV1-CoV), and Human coronavirus HKU9 (HKU9-CoV). Non-limiting examples of alphacoronaviruses include human coronavirus 229E (229E-CoV), human coronavirus NL63 (NL63-CoV), porcine epidemic diarrhea virus (PEDV), and Transmissible gastroenteritis coronavirus (TGEV). A non-limiting example of a deltacoronaviruses is the Swine Delta Coronavirus (SDCV).

A list of Severe acute respiratory syndrome-related coronavirus is disclosed herein:
Bat coronavirus Cp/Yunnan2011
Bat coronavirus RaTG13
Bat coronavirus Rp/Shaanxi2011
Bat SARS coronavirus IHKU3
   Bat SARS coronavirus HKU3-1
   Bat SARS coronavirus HKIU3-10
   Bat SARS coronavirus HKU3-11
   Bat SARS coronavirus HKU3-12
   Bat SARS coronavirus HKU3-13
   Bat SARS coronavirus HKU3-2
   Bat SARS coronavirus HKU3-3
   Bat SARS coronavirus HKU3-4
   Bat SARS coronavirus HKU3-5
   Bat SARS coronavirus HKU3-6
   Bat SARS coronavirus HKU3-7
   Bat SARS coronavirus HKU3-8
   Bat SARS coronavirus HKU3-9
Bat SARS coronavirus Rp1
Bat SARS coronavirus Rp2
Bat SARS CoV Rf1/2004
   Bat CoV 273/2005
Bat SARS CoV Rm1/2004
   Bat CoV 279/2005
Bat SARS CoV Rp3/2004
Bat SARS-like coronavirus
Bat SARS-like coronavirus Rs3367
Bat SARS-like coronavirus RsSHC014
Bat SARS-like coronavirus WIV1
Bat SARS-like coronavirus YNLF_31C
Bat SARS-like coronavirus YNLF_34C
BtRf-BetaCoV/HeB2013
BtRf-BetaCoV/JL2012
BtRf-BetaCoV/SX2013
BtRs-BetaCoV/GX2013
BtRs-BetaCoV/HuB2013
BtRs-BetaCoV/YN2013
Civet SARS CoV 007/2004
Civet SARS CoV SZ16/2003
Civet SARS CoV SZ3/2003
recombinant SARSr-CoV
   SARS coronavirus ExoN1
   SARS coronavirus MA15
   SARS coronavirus MA15 ExoN1
   SARS coronavirus wtic-MB
Rhinolophus affinis coronavirus
SARS bat coronavirus
SARS coronavirus A001
SARS coronavirus A013
SARS coronavirus A021
SARS coronavirus A022
SARS coronavirus A030
SARS coronavirus A031
SARS coronavirus AS
SARS coronavirus B012
SARS coronavirus B024
SARS coronavirus B029
SARS coronavirus B033
SARS coronavirus B039
SARS coronavirus B040
SARS coronavirus BJ01
SARS coronavirus BJ02
SARS coronavirus BJ03
SARS coronavirus BJ04
SARS coronavirus BJJ162
SARS coronavirus BJ182-12
SARS coronavirus BJ182-4
SARS coronavirus BJ182-8
SARS coronavirus BJ182a
SARS coronavirus BJ182b
SARS coronavirus BJ202
SARS coronavirus BJ2232
SARS coronavirus BJ302
SARS coronavirus C013
SARS coronavirus C014
SARS coronavirus C017
SARS coronavirus C018
SARS coronavirus C019
SARS coronavirus C025
SARS coronavirus C028
SARS coronavirus C029
SARS Coronavinis CDC #200301157
SARS coronavirus civet010
SARS coronavirus civet014
SARS coronavirus civet019
SARS coronavirus civet020
SARS coronavirus CS21
SARS coronavirus CS24
SARS coronavirus CUHK-AG01
SARS coronavirus CUHK-AGO2
SARS coronavirus CUHK-AG03
SARS coronavirus CUHK-L2
SARS coronavirus CUHK-Su10
SARS coronavirus CLHK-W1
SARS coronavirus cwt037
SARS coronavirus cwt049
SARS coronavirus ES191
SARS coronavirus ES260
SARS coronavirus FRA
SARS coronavirus Frankfurt 1
   SARS coronavirus Frankfurt1-v01
SARS coronavirus GD01
SARS coronavirus GD03T0013
SARS coronavirus GD322
SARS coronavirus GD69
SARS coronavirus GDH-BJH01
SARS coronavirus GZ-A
SARS coronavirus GZ-B
SARS coronavirus GZ-C
SARS coronavirus GZ-D
SARS coronavirus GZ02
SARS coronavirus GZ0401
SARS coronavirus GZ0402
SARS coronavirus GZ0403
SARS coronavirus GZ43
SARS coronavirus GZ50
SARS coronavirus GZ60
SARS coronavirus HB
SARS coronavirus HC/SZ/61/03
SARS coronavirus HGZ8L1-A
SARS coronavirus HIGZ8L1-B
SARS coronavirus HGZ8L2
SARS coronavirus HHS-2004
SARS coronavirus HKU-36871
SARS coronavirus HKU-39849
SARS coronavirus HKU-65806
SARS coronavirus HKU-66078
SARS coronavirus Hong Kong/03/2003
SARS coronavirus HPZ-2003
SARS coronavirus HSR 1
SARS coronavirus HSZ-A
SARS coronavirus HSZ-Bb
SARS coronavirus HSZ-Bc
SARS coronavirus HSZ-Cb
SARS coronavirus HSZ-Cc
SARS coronavirus HSZ2-A
SARS coronavirus HZS2-Bb
SARS coronavirus HZS2-C
SARS coronavirus HZS2-D
SARS coronavirus HZS2-E
SARS coronavirus HZS2-Fb
SARS coronavirus HZS2-Fc
SARS coronavirus JMD
SARS coronavirus LC1
SARS coronavirus LC2
SARS coronavirus LC3
SARS coronavirus LC4
SARS coronavirus LC5
SARS coronavirus LLJ-2004
SARS coronavirus NS-1
SARS coronavirus P2
SARS coronavirus PC4-115
SARS coronavirus PC4-127
SARS coronavirus PC4-13
SARS coronavirus PC4-136
SARS coronavirus PC4-137
SARS coronavirus PC4-145
SARS coronavirus PC4-199
SARS coronavirus PC4-205
SARS coronavirus PC4-227
SARS coronavirus PC4-241
SARS coronavirus PUMCO1
SARS coronavirus PUMC02
SARS coronavirus PUMC03
SARS coronavirus Rs_672/2006
SARS coronavirus sf098
SARS coronavirus sf099
SARS coronavirus ShanghaiQXC1
SARS coronavirus ShanghaiQXC2
SARS coronavirus Shanhuai LY
SARS coronavirus Sin0409
SARS coronavirus Sin2500
SARS coronavirus Sin2677
SARS coronavirus Sin2679
SARS coronavirus Sin2748
SARS coronavirus Sin2774
SARS coronavirus Sin3408
SARS coronavirus Sin3408L
SARS coronavirus Sin3725V
SARS coronavirus Sin3765V
SARS coronavirus Sin842
SARS coronavirus Sin845
SARS coronavirus Sin846
SARS coronavirus Sin847
SARS coronavirus Sin848
SARS coronavirus Sin849
SARS coronavirus Sin850
SARS coronavirus Sin852
SARS coronavirus Sin_WNV
SARS coronavirus Sino1-11
SARS coronavirus Sino3-11
SARS coronavirus SinP1
SARS coronavirus SinP2
SARS coronavirus SinP3
SARS coronavirus SinP4
SARS coronavirus SinP5
SARS coronavirus SoD
SARS coronavirus SZ1
SARS coronavirus SZ13
SARS coronavirus Taiwan
SARS coronavirus Taiwan JC-2003
SARS coronavirus Taiwan TC
SARS coronavirus Taiwan TC2
SARS coronavirus Taiwan TC3
SARS coronavirus TJ01
SARS coronavirus TJF
SARS coronavirus Tor2
SARS coronavirus TW
   SARS coronavirus TW-GD1
   SARS coronavirus TW-GD2
   SARS coronavirus TW-GD3
   SARS coronavirus TW-GD4
   SARS coronavirus TW-GD5
   SARS coronavirus TW—HP1
   SARS coronavirus TW-HP2
   SARS coronavirus TW-HP3
   SARS coronavirus TW-HP4
   SARS coronavirus TW-JC2
   SARS coronavirus TW-KC1
   SARS coronavirus TW-KC3
   SARS coronavirus TV-PH1
   SARS coronavirus TW-PH2
   SARS coronavirus TW-YM1
   SARS coronavirus TW-YM2
   SARS coronavirus TW-YM3
   SARS coronavirus TW-YM4
SARS coronavirus TW1
SARS coronavirus TW10
SARS coronavirus TW11
SARS coronavirus TW2
SARS coronavirus TW3
SARS coronavirus TW4
SARS coronavirus TW5
SARS coronavirus TW6
SARS coronavirus TW7
SARS coronavirus TW8
SARS coronavirus TW9
SARS coronavirus TWC
SARS coronavirus TWC2
SARS coronavirus TWC3
SARS coronavirus TWH
SARS coronavirus TWJ
SARS coronavirus TWK
SARS coronavirus TWS
SARS coronavirus TWY
SARS coronavirus Urbani
SARS coronavirus Vietnam
SARS coronavirus WF188
SARS coronavirus WH20
SARS coronavirus WHU
SARS coronavirus xw002
SARS coronavirus ZJ01
SARS coronavirus ZJ02
SARS coronavirus ZJ0301
SARS coronavirus ZMY 1
SARS coronavirus ZS-A
SARS coronavirus ZS-B
SARS coronavirus ZS-C
SARS-related bat coronavirus RsSHC014
SARS-related betacoronavirus Rp3/2004
Severe acute respiratory syndrome coronavirus 2

Exemplary SARS CoV-2 strains are shown in the table below.

| | | | Notable | | |
|---|---|---|---|---|---|
| Name/Designation | | Distribution | Mutation(s) | Impact | Sequence |
| D614G | | Worldwide | D614G | Increased | P0DTC2 |
| | | | | infectivity, | |
| | | | | Dominant | |
| | | | | circulating | |
| | | | | since June 2020 | |
| B.1.1.7 | 501Y.V1 | UK/Worldwide | D614G, N501Y, | Increased | B.1.1.7 |
| | | (nearly dominant | P681H | infectivity | Lineages |
| | | in US) | | | |
| B.1.351 | 501.V2, or | South Africa | N501Y, | Increased | B.1.351 |
| | N501Y.V2 | | E484K*, | infectivity, | Lineages |
| | | | K417N | *escape | |
| | | | | mutation* | |
| B. 1.1.248 | P1 | Brazil | N501Y, | Increased | P1 Lineages |
| | | | E484K*, K417T | infectivity, | |
| | | | | *escape | |
| | | | | mutation* | |

The coronavirus viral genome is capped, polyadenylated, and covered with nucleocapsid proteins. The coronavirus virion includes a viral envelope containing type I fusion glycoproteins referred to as the spike (S) protein. Most coronaviruses have a common genome organization with the replicase gene included in the 5′-portion of the genome, and structural genes included in the 3′-portion of the genome.

Coronavirus Spike (S) protein is class I fusion glycoprotein initially synthesized as a precursor protein. Individual precursor S polypeptides form a homotrimer and undergo glycosylation within the Golgi apparatus as well as processing to remove the signal peptide, and cleavage by a cellular protease to generate separate S1 and S2 polypeptide chains, which remain associated as S1/S2 protomers within the homotrimer and is therefore a trimer of heterodimers. The S1 subunit is distal to the virus membrane and contains the receptor-binding domain (RBD) that mediates virus attachment to its host receptor. The S2 subunit contains fusion protein machinery, such as the fusion peptide, two heptad-repeat sequences (HR1 and HR2) and a central helix typical of fusion glycoproteins, a transmembrane domain, and the cytosolic tail domain.

In some cases, the coronavirus viral antigen or immunogen is a coronavirus S protein peptide in a prefusion conformation, which is a structural conformation adopted by the ectodomain of the coronavirus S protein following processing into a mature coronavirus S protein in the secretory system, and prior to triggering of the fusogenic event that leads to transition of coronavirus S to the postfusion conformation. The three-dimensional structure of an exemplary coronavirus S protein (HKU1-CoV) in a prefusion conformation is provided in Kirchdoerfer et al., “Pre-fusion structure of a human coronavirus spike protein,” Nature, 531: 118-121, 2016.

In some cases, the coronavirus viral antigen or immunogen comprises one or more amino acid substitutions, deletions, or insertions compared to a native coronavirus S sequence that provide for increased retention of the prefusion conformation compared to coronavirus S ectodomain trimers formed from a corresponding native coronavirus S sequence. The “stabilization” of the prefusion conformation by the one or more amino acid substitutions, deletions, or insertions can be, for example, energetic stabilization (for example, reducing the energy of the prefusion conformation relative to the post-fusion open conformation) and/or kinetic stabilization (for example, reducing the rate of transition from the prefusion conformation to the postfusion conformation). Additionally, stabilization of the coronavirus S ectodomain trimer in the prefusion conformation can include an increase in resistance to denaturation compared to a corresponding native coronavirus S sequence. Methods of determining if a coronavirus S ectodomain trimer is in the prefusion conformation are provided herein, and include (but are not limited to) negative-stain electron microscopy and antibody binding assays using a prefusion-conformation-specific antibody.

In some cases, the coronavirus viral antigen or immunogen is a fragment of an S protein peptide. In some embodiments, the antigen or immunogen is an epitope of an S protein peptide. Epitopes include antigenic determinant chemical groups or peptide sequences on a molecule that are antigenic, such that they elicit a specific immune response, for example, an epitope is the region of an antigen to which B and/or T cells respond. An antibody can bind to a particular antigenic epitope, such as an epitope on coronavirus S ectodomain. Epitopes can be formed both from contiguous amino acids or noncontiguous amino acids juxtaposed by tertiary folding of a protein. In some embodiments, the coronavirus epitope is a linear epitope. In some embodiments, the coronavirus epitope is a conformational epitope. In some embodiments, the coronavirus epitope is a neutralizing epitope site. In some embodiments, all neutralizing epitopes of the coronavirus S protein peptide or fragment thereof are present as the antigen or immunogen.

In some cases, for example when the viral antigen or immunogen is a fragment of an S protein peptide, only a single subunit of the S protein peptide is present, and that single subunit of the S protein peptide is trimerized. In some embodiments, the viral antigen or immunogen comprises a signal peptide, an S1 subunit peptide, an S2 subunit peptide, or any combination thereof. In some embodiments, the viral antigen or immunogen comprises a signal peptide, a receptor binding domain (RBD) peptide, a receptor binding motif (RBM) peptide, a fusion peptide (FP), a heptad repeat 1 (HR1) peptide, or a heptad repeat 2 (HR2) peptide, or any combination thereof. In some embodiments, the viral antigen or immunogen comprises a receptor binding domain (RBD) of the S protein. In some embodiments, the viral antigen or immunogen comprises an S1 subunit and an S2 subunit of the S protein. In some embodiments, the viral antigen or immunogen comprises an S1 subunit of the S protein but not an S2 subunit. In some embodiments, the viral antigen or immunogen comprises an S2 subunit of the S protein but not an S1 subunit. In some embodiments, the viral antigen or immunogen is free of a transmembrane (TM) domain peptide and/or a cytoplasm (CP) domain peptide.

In some embodiments, the viral antigen or immunogen comprises a protease cleavage site, wherein the protease is optionally furin, trypsin, factor Xa, or cathepsin L.

In some embodiments, the viral antigen or immunogen is free of a protease cleavage site, wherein the protease is optionally furin, trypsin, factor Xa, or cathepsin L, or contains a mutated protease cleavage site that is not cleavable by the protease.

In some embodiments, the viral antigen or immunogen is a SARS-CoV-2 antigen comprising at least one SARS-CoV-2 protein or fragment thereof. In some embodiments, the SARS-CoV-2 antigen is recognized by SARS-CoV-2 reactive antibodies and/or T cells. In some embodiments, the SARS-CoV-2 antigen is an inactivated whole virus. In some embodiments, the SARS-CoV-2 antigen comprises is a subunit of the virus. In some embodiments, the SARS-CoV-2 antigen comprises a structural protein of SARS-CoV-2 or a fragment thereof. In some embodiments, the structural protein of SARS-CoV-2 comprises one or more of the group consisting of the spike (S) protein, the membrane (M) protein, nucleocapsid (N) protein, and envelope (E) protein. In some embodiments, the SARS-CoV-2 antigen comprises or further comprises a non-structural protein of SARS-CoV-2 or a fragment thereof. The nucleotide sequence of a representative SARS-CoV-2 isolate (Wuhan-Hu-1) is set forth as GenBank No. MN908947.3 (Wu et al., Nature, 579:265-269, 2020).

In some embodiments, the viral antigen or immunogen comprises the sequence set forth in SEQ ID NO: 55. In some embodiments, the viral antigen or immunogen comprises an amino acid sequence having at least or about 80%, 85%, 90%, 92%, 95%, or 97% sequence identity to sequence of SEQ ID NO: 55 shown below (underlined sequence indicating the receptor-binding motif (RBM) within the receptor binding domain (RBD) from Thr333-Gly526, bolded). In some embodiments, the viral antigen or immunogen comprises an RBD-Trimer, for example, a SARS-CoV-2 RBD sequence linked to any of SEQ ID Nos: 67-80.

```
                10 20 30 40 50 60
MFVFLVLLPLVSSQCVNLTTRTQLPPAYTNSFTRGVYYPDKVFRSSVLHSTQDLFLPFFS
                70 80 90 100 110 120
NVTWFHAIHVSGTNGTKRFDNPVLPFNDGVYFASTEKSNIIRGWIFGTTLDSKTQSLLIV
              130 140 150 160 170 180
NNATNVVIKVCEFQFCNDPFLGVYYHKNNKSWMESEFRVYSSANNCTFEYVSQPFLMDLE
              190 200 210 220 230 240
GKQGNFKNLREFVFKNIDGYFKIYSKHTPINLVRDLPQGFSALEPLVDLPIGINITRFQT
              250 260 270 280 290 300
LLALHRSYLTPGDSSSGWTAGAAAYYVGYLQPRTFLLKYNENGTITDAVDCALDPLSETK
              310 320 330 340 350 360
CTLKSFTVEKGIYQTSNFRVQPTESIVRFPNITNLCPFGEVFNATRFASVYAWNRKRISN
              370 380 390 400 410 420
CVADYSVLYNSASFSTFKCYGVSPTKLNDLCFTNVYADSFVIRGDEVRQIAPGQTGKIAD
              430 440 450 460 470 480
YNYKLPDDFTGCVIAWNSNNLDSKVGGNYNYLYRLFRKSNLKPFERDISTEIYQAGSTPC
              490 500 510 520 530 540
NGVEGFNCYFPLQSYGFQPTNGVGYQPYRVVVLSFELLHAPATVCGPKKSTNLVKNKCVN
              550 560 570 580 590 600
FNFNGLTGTGVLTESNKKFLPFQQFGRDIADTTDAVRDPQTLEILDITPCSFGGVSVITP
              610 620 630 640 650 660
GTNTSNQVAVLYQDVNCTEVPVAIHADQLTPTWRVYSTGSNVFQTRAGCLIGAEHVNNSY
              670 680 690 700 710 720
ECDIPIGAGICASYQTQTNSPRRARSVASQSIIAYTMSLGAENSVAYSNNSIAIPTNFTI
              730 740 750 760 770 780
SVTTEILPVSMTKTSVDCTMYICGDSTECSNLLLQYGSFCTQLNRALTGIAVEQDKNTQE
              790 800 810 820 830 840
VFAQVKQIYKTPPIKDFGGFNFSQILPDPSKPSKRSFIEDLLFNKVTLADAGFIKQYGDC
              850 860 870 880 890 900
LGDIAARDLICAQKFNGLTVLPPLLTDEMIAQYTSALLAGTITSGWTFGAGAALQIPFAM
              910 920 930 940 950 960
QMAYRFNGIGVTQNVLYENQKLIANQFNSAIGKIQDSLSSTASALGKLQDVVNQNAQALN
              970 980 990 1000 1010 1020
TLVKQLSSNFGAISSVLNDILSRLDKVEAEVQIDRLITGRLQSLQTYVTQQLIRAAEIRA
            1030 1040 1050 1060 1070 1080
SANLAATKMSECVLGQSKRVDFCGKGYRLMSFPQSAPHGVVFLHVTYVPAQEKNFTTAPA
            1090 1100 1110 1120 1130 1140
ICHDGKAHEPREGVFVSNGTHWFVIQRNFYEPQIITTDNTFVSGNCDVVLGLVNNTVYDP
            1150 1160 1170 1180 1190 1200
LQPELDSFKEELDKYFKNHTSPDVDLGDISGINASVVNIQKEIDRLNEVAKNLNESLIDL
            1210 1220 1230 1240 1250 1260
QELGKYEQYIKWPWYIWLGFIAGLIAIVMVTIMLCCMTSCCSCLKGCCSCGSCCKFDEDD
            1270
SEPVLKGVKLHYT
```

In some embodiments, the viral antigen or immunogen comprises a sequence of the spike glycoprotein of the original Wuhan-Hu-1 coronavirus (e.g., NC_045512). In some embodiments, the viral antigen or immunogen comprises a sequence of the spike glycoprotein of a virus in the B.1.526 lineage. In some embodiments, the viral antigen or immunogen comprises a sequence of the spike glycoprotein of a Cluster 5 (ΔFVI-spike) virus. In some embodiments, the viral antigen or immunogen comprises a sequence of the spike glycoprotein of a virus in the B.1.1.7 lineage. In some embodiments, the viral antigen or immunogen comprises a sequence of the spike glycoprotein of a virus in the B.1.1.207 lineage. In some embodiments, the viral antigen or immunogen comprises a sequence of the spike glycoprotein of a virus in the B.1.1.317 lineage. In some embodiments, the viral antigen or immunogen comprises a sequence of the spike glycoprotein of a virus in the B.1.1.318 lineage. In some embodiments, the viral antigen or immunogen comprises a sequence of the spike glycoprotein of a virus in the P.1 lineage. In some embodiments, the viral antigen or immunogen comprises a sequence of the spike glycoprotein of a virus in the B.1.351 lineage. In some embodiments, the viral antigen or immunogen comprises a sequence of the spike glycoprotein of a virus in the B.1.429/CAL.20C lineage. In some embodiments, the viral antigen or immunogen comprises a sequence of the spike glycoprotein of a virus in the B.1.525 lineage. In some embodiments, the viral antigen or immunogen comprises a sequence of the spike glycoprotein of a virus in the B.1.526 lineage. In some embodiments, the viral antigen or immunogen comprises a sequence of the spike glycoprotein of a virus in the B.1.617 lineage. In some embodiments, the viral antigen or immunogen comprises a sequence of the spike glycoprotein of a virus in the B.1.617.2 lineage. In some embodiments, the viral antigen or immunogen comprises a sequence of the spike glycoprotein of a virus in the B.1.618 lineage. In some embodiments, the viral antigen or immunogen comprises a sequence of the spike glycoprotein of a virus in the B.1.620 lineage. In some embodiments, the viral antigen or immunogen comprises a sequence of the spike glycoprotein of a virus in the P.2 lineage. In some embodiments, the viral antigen or immunogen comprises a sequence of the spike glycoprotein of a virus in the P.3 lineage. In some embodiments, the viral antigen or immunogen comprises a sequence of the spike glycoprotein of a virus in the B.1.1.143 lineage. In some embodiments, the viral antigen or immunogen comprises a sequence of the spike glycoprotein of a virus in the A.23.1 lineage. In some embodiments, the viral antigen or immunogen comprises a sequence of the spike glycoprotein of a virus in the B.1.617 lineage. In some embodiments, the viral antigen or immunogen comprises sequences derived from the spike glycoproteins of any two or more viruses, in any suitable combination, selected from the group consisting of Wuhan-Hu-1, a virus in the B.1.526 lineage, a virus in the B.1.1.7 lineage, a virus in the P.1 lineage, a virus in the B.1.351 lineage, a virus in the P.2 lineage, a virus in the B.1.1.143 lineage, a virus in the A.23.1 lineage, and a virus in the B.1.617 lineage.

In some embodiments, the viral antigen or immunogen comprises E484K and/or S477N, e.g., as in a B.1.526 variant. In some embodiments, the viral antigen or immunogen comprises Δ400-402 (ΔFVI), e.g., as in a Cluster 5 (ΔFVI-spike) variant. In some embodiments, the viral antigen or immunogen comprises Δ69-70 (ΔHV), Δ144 (ΔY), N501Y, A570D, D614G, P681H, T716I, S982A, and/or D118H, e.g., as in a B.1.1.7 variant. In some embodiments, the viral antigen or immunogen comprises P681H, e.g., as in a B.1.1.207 variant. In some embodiments, the viral antigen or immunogen comprises L18F, T20N, P26S, D138Y, R190S, K417T, E484K, N501Y, D614G, H655Y, Ti0271, and/or V1176F, e.g., as in a P.1 variant. In some embodiments, the viral antigen or immunogen comprises E484K, e.g., as in a P.2 variant. In some embodiments, the viral antigen or immunogen comprises E484K and/or N501Y, e.g., as in a P.3 variant. In some embodiments, the viral antigen or immunogen comprises L18F, D80A, D215G, Δ242-244 (ΔLAL), R246I, K417N, E484K, N501Y, D614G, and/or A701V, e.g., as in a B.1.351 variant. In some embodiments, the viral antigen or immunogen comprises S13I, W152C, and/or L452R, e.g., as in a B.1.429/CAL.20C variant. In some embodiments, the viral antigen or immunogen comprises Δ69-70 (ΔHV), E484K, and/or F888L, e.g., as in a B.1.525 variant. In some embodiments, the viral antigen or immunogen comprises G142D, L452R, E484Q, and/or P681R, e.g., as in a B.1.617 variant. In some embodiments, the viral antigen or immunogen comprises G142D, L452R, and/or P681R, e.g., as in a B.1.617.2 variant. In some embodiments, the viral antigen or immunogen comprises E484K, e.g., as in a B.1.618 variant. In some embodiments, the viral antigen or immunogen may comprise a fusion polypeptide (protomer) comprising any one or more of the aforementioned mutations in any suitable combination. In some embodiments, the viral antigen or immunogen may comprise a trimer of three fusion polypeptides, and any of the three protomer fusion polypeptides may comprise any one or more of the aforementioned mutations in any suitable combination. In some embodiments, two or all three of the three protomer fusion polypeptides forming a trimer may comprise different mutations and/or different combinations of mutations in each protomer. In some embodiments, the viral antigen or immunogen may comprise a mixture of trimers, and each trimer may comprise different mutations and/or different combinations of mutations.

In some embodiments, the viral antigen or immunogen comprises any one, two, three, four, five or more of the mutations selected from the group consisting of mutations (e.g., substitution(s), deletion(s) and/or insertion(s)) at amino acid positions 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 of SEQ ID NO: 55. In some embodiments, the viral antigen or immunogen comprises any one, two, three, four, five, six, seven, eight, or all of the mutations selected from the group consisting of mutations (e.g., substitution(s), deletion(s) and/or insertion(s)) at amino acid positions 440, 452, 477, 484, 501, 614, 655, 681, and 701. In some embodiments, the viral antigen or immunogen comprises a chimeric polypeptide comprising sequences from different viruses, such as one or more mutations from a first variant of a coronavirus and one or more mutations from a second variant of the coronavirus that is different from the first variant. In some embodiments, such a chimeric viral antigen or immunogen (or a combination of chimeric viral antigens or immunogens) may be used to elicit a broad immune response against both the first and second variants of the coronavirus. In some embodiments, such a chimeric viral antigen or immunogen (or a combination of chimeric viral antigens or immunogens) may be used as an antigen for sensitive detection of an analyte (e.g., SARS-CoV-2 antibodies such as IgG, IgM, and/or IgE that neutralize the virus) that binds to the viral antigen or immunogen, e.g., in an ELISA or lateral flow assay.

In some embodiments, the viral antigen or immunogen comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681K P681R, R682G, R683S, R685G, A701V, T716I, F888L, S982A, T1027I, D1118H, and V1176F. In some embodiments, the viral antigen or immunogen comprises any one, two, three, four, five or more of the mutations selected from the group consisting of N440K, L452R, S477G, S477N, E484K, E484Q, N501Y, D614G, H655Y, P681H, P681R, and A701V.

In some embodiments, the SARS-CoV-2 antigen comprises a truncated, S protein devoid of signal peptide, transmembrane and cytoplasmic domains of a full length S protein. In some embodiments, the SARS-CoV-2 antigen is a recombinant protein, while in other embodiments, the SARS-CoV-2 antigen is purified from virions. In some preferred embodiments, the SARS-CoV-2 antigen is an isolated antigen.

In some embodiments, the viral antigen or immunogen comprises the sequence set forth in SEQ ID NO: 27. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 27, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 27 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F.

In some embodiments, the viral antigen or immunogen comprises the sequence set forth in SEQ ID NO: 28. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 28, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 28 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F.

In some embodiments, the viral antigen or immunogen comprises the sequence set forth in SEQ ID NO: 29. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 29, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 29 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F.

In some embodiments, the viral antigen or immunogen comprises the sequence set forth in SEQ ID NO: 30. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 30, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 30 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118K and V1176F.

In some embodiments, the viral antigen or immunogen comprises the sequence set forth in SEQ ID NO: 31. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 31, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 31 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F.

In some embodiments, the viral antigen or immunogen comprises the sequence set forth in SEQ ID NO: 32. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 32, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 32 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118K and V1176F.

In some embodiments, the viral antigen or immunogen comprises the sequence set forth in SEQ ID NO: 33. In some embodiments, the viral antigen or immunogen comprises an amino acid sequence having at least or about 80%, 81%, 82%, 83%, 84%, 85%, 86%, 87%, 88%, 890/c, 90%, 91%, 92%, 93%, 94%, 95%, 96%, 97%, 98%, or 99% sequence identity to sequence of SEQ ID NO: 33, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 33 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F.

In some embodiments, the viral antigen or immunogen comprises the sequence set forth in SEQ ID NO: 34. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 34, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 34 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118K and V1176F.

In some embodiments, the viral antigen or immunogen comprises the sequence set forth in SEQ ID NO: 35. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 35, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 35 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D118H, and V1176F.

In some embodiments, the viral antigen or immunogen comprises the sequence set forth in SEQ ID NO: 36. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 36, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 36 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, RI90S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F.

In some embodiments, the viral antigen or immunogen comprises the sequence set forth in SEQ ID NO: 37. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 37, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 37 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D118H, and V1176F.

In some embodiments, the viral antigen or immunogen comprises the sequence set forth in SEQ ID NO: 38. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 38, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 38 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, RI90S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F.

In some embodiments, the viral antigen or immunogen comprises the sequence set forth in SEQ ID NO: 39. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 39, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 39 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, RI90S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D118H, and V1176F.

In some embodiments, the viral antigen or immunogen comprises the sequence set forth in SEQ ID NO: 40. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 40, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 40 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F.

In some embodiments, the viral antigen or immunogen comprises the sequence set forth in SEQ ID NO: 41. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 41, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 41 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F.

In some embodiments, the viral antigen or immunogen comprises the sequence set forth in SEQ ID NO: 42. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 42, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 42 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F.

In some embodiments, the viral antigen or immunogen comprises the sequence set forth in SEQ ID NO: 43. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 43, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 43 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F.

In some embodiments, the viral antigen or immunogen comprises the sequence set forth in SEQ ID NO: 44. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 44, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 44 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F.

In some embodiments, the viral antigen or immunogen comprises the sequence set forth in SEQ ID NO: 45. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 45, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 45 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118K and V1176F.

In some embodiments, the viral antigen or immunogen comprises the sequence set forth in SEQ ID NO: 46. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 46, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 46 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F.

In some embodiments, the viral antigen or immunogen comprises the sequence set forth in SEQ ID NO: 47. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 47, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 47 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118K and V1176F.

In some embodiments, the viral antigen or immunogen comprises the sequence set forth in SEQ ID NO: 48. In some embodiments, the viral antigen or immunogen comprises an amino acid sequence having at least or about 80%, 81%, 82%, 83%, 84%, 85%, 86%, 87%, 88%, 890/c, 90%, 91%, 92%, 93%, 94%, 95%, 96%, 97%, 98%, or 99% sequence identity to sequence of SEQ ID NO: 48, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 48 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F.

In some embodiments, the viral antigen or immunogen comprises the sequence set forth in SEQ ID NO: 49. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 49, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 49 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118K and V1176F.

In some embodiments, the viral antigen or immunogen comprises the sequence set forth in SEQ ID NO: 50. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 50, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 50 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D118H, and V1176F.

In some embodiments, the viral antigen or immunogen comprises the sequence set forth in SEQ ID NO: 51. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 51, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 51 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F.

In some embodiments, the viral antigen or immunogen comprises the sequence set forth in SEQ ID NO: 52. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 52.

In some embodiments, the viral antigen or immunogen comprises a signal peptide. In some embodiments, the viral antigen or immunogen comprises the sequence set forth in SEQ ID NO: 53. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 53. In some embodiments, the viral antigen or immunogen comprises the sequence set forth in SEQ ID NO: 54. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 54.

In some embodiments, the viral antigen or immunogen comprises the sequence set forth in SEQ ID NO: 55. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 55, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176. In some embodiments, the viral antigen or immunogen comprises a variant of SEQ ID NO: 55 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, Ti0271, D1118H, and V1176F.

In some embodiments, the viral antigen or immunogen comprises the sequence set forth in SEQ ID NO: 56. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 56, including a sequence comprising substitution, deletion, and/or insertion at one or more amino acid positions selected from the group consisting of 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and 1176 (amino acid positions with respect to SEQ ID NO: 55). In some embodiments, the viral antigen or immunogen comprises a variant of SEQ ID NO: 56 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, RI90S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D118H, and V1176F.

In some embodiments, the viral antigen or immunogen comprises the sequence set forth in SEQ ID NO: 57. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 57, including a sequence comprising substitution, deletion, and/or insertion at one or more amino acid positions of SEQ ID NO: 57.

In some embodiments, the viral antigen or immunogen comprises the sequence set forth in SEQ ID NO: 58. In some embodiments, the viral antigen or immunogen comprises an amino acid sequence having at least or about 80%, 81%, 82%%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 58, including a sequence comprising substitution, deletion, and/or insertion at one or more amino acid positions of SEQ ID NO: 58.

In some embodiments, the viral antigen or immunogen comprises the sequence set forth in SEQ ID NO: 59. In some embodiments, the viral antigen or immunogen comprises an amino acid sequence comprising substitution, deletion, and/or insertion at one or more amino acid positions of SEQ ID NO: 59. In some embodiments, the viral antigen or immunogen comprises the sequence set forth in SEQ ID NO: 60. In some embodiments, the viral antigen or immunogen comprises an amino acid sequence comprising substitution, deletion, and/or insertion at one or more amino acid positions of SEQ ID NO: 60.

In some embodiments, the viral antigen or immunogen comprises the sequence set forth in SEQ ID NO: 61. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 61, including a sequence comprising substitution, deletion, and/or insertion at one or more amino acid positions of SEQ ID NO: 61.

In some embodiments, the viral antigen or immunogen comprises the sequence set forth in SEQ ID NO: 62. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 62, including a sequence comprising substitution, deletion, and/or insertion at one or more amino acid positions of SEQ ID NO: 62.

In some embodiments, the viral antigen or immunogen comprises the sequence set forth in SEQ ID NO: 63. In some embodiments, the viral antigen or immunogen comprises an amino acid sequence having at least or about 80%, 81%, 82%%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 63, including a sequence comprising substitution, deletion, and/or insertion at one or more amino acid positions of SEQ ID NO: 63.

In some embodiments, the viral antigen or immunogen comprises the sequence set forth in SEQ ID NO: 64. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 64, including a sequence comprising substitution, deletion, and/or insertion at one or more amino acid positions of SEQ ID NO: 64.

In some embodiments, the viral antigen or immunogen comprises the sequence set forth in SEQ ID NO: 65. In some embodiments, the viral antigen or immunogen comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to sequence of SEQ ID NO: 65, including a sequence comprising substitution, deletion, and/or insertion at one or more amino acid positions of SEQ ID NO: 65.

In some embodiments, the viral antigen or immunogen does not comprise a transmembrane domain such as SEQ ID NO: 66 or a portion thereof. In some embodiments, the coronavirus viral antigen or immunogen comprises an S protein peptide that is soluble. In some embodiments, the soluble S protein peptide lacks a TM domain peptide and a CP domain peptide. In some embodiments, the soluble S protein peptide does not bind to a lipid bilayer, such as a membrane or viral envelope.

In some embodiments, the S protein peptide is produced from a nucleic acid sequence that has been codon optimized. In some embodiments, the S protein peptide is produced from a nucleic acid sequence that has not been codon optimized.

In some embodiments, the viral antigen or immunogen as referred to herein can include recombinant polypeptides or fusion peptides comprising said viral antigen or immunogen. The terms viral antigen or immunogen may be used to refer to proteins comprising a coronavirus viral antigen or immunogen. In certain cases, the coronavirus viral antigen or immunogen is a coronavirus protein peptide as provided herein.

### II. Recombinant Peptides and Proteins

It is contemplated that the coronavirus viral antigens and immunogens provided herein, e.g., S protein peptides (see, Section I), can be combined, e.g., linked, to other proteins or peptides to form recombinant polypeptides, including fusion peptides. In some embodiments, individual recombinant polypeptides (e.g., monomers) provided herein associate to form multimers, e.g., trimers, of recombinant polypeptides. In some embodiments, association of the individual recombinant polypeptide monomers occurs via covalent interactions. In some embodiments, association of the individual recombinant polypeptide monomers occurs via non-covalent interactions. In some embodiments, the interaction, e.g., covalent or non-covalent, is effected by the protein or peptide to which the coronavirus viral antigen or immunogen, e.g., S protein peptide, is linked. In some embodiments, for example when the coronavirus viral antigen or immunogen is an S protein peptide as described herein, the protein or peptide to which it will be linked can be selected such that the native homotrimeric structure of the glycoprotein is preserved. This can be advantageous for evoking a strong and effective immunogenic response to the S protein peptide. For example, preservation and/or maintenance of the native conformation of the coronavirus viral antigens or immunogens (e.g., S protein peptide) may improve or allow access to antigenic sites capable to generating an immune response. In some cases, the recombinant polypeptide comprising an S protein peptide described herein, e.g., see Section I, is referred to herein alternatively as a recombinant S antigen, recombinant S immunogen, or a recombinant S protein.

It is further contemplated that in some cases, the recombinant polypeptides or multimerized recombinant polypeptides thereof aggregate or can be aggregated to form a protein or a complex comprising a plurality of coronavirus viral antigen and/or immunogen recombinant polypeptides. Formation of such proteins may be advantageous for generating a strong and effective immunogenic response to the coronavirus viral antigens and/or immunogens. For instance, formation of a protein comprising a plurality of recombinant polypeptides, and thus a plurality of coronavirus viral antigens, e.g., coronavirus S protein peptides, may preserve the tertiary and/or quaternary structures of the viral antigen, allowing an immune response to be mounted against the native structure. In some cases, the aggregation may confer structural stability of the coronavirus viral antigen or immunogen, which in turn can afford access to potentially antigenic sites capable of promoting an immune response.

In some embodiments, the coronavirus viral antigen or immunogen can be linked at their C-terminus (C-terminal linkage) to a trimerization domain to promote trimerization of the monomers. In some embodiments, the trimerization stabilizes the membrane proximal aspect of the coronavirus viral antigen or immunogen, e.g., coronavirus S protein peptide, in a trimeric configuration.

Non-limiting examples of exogenous multimerization domains that promote stable trimers of soluble recombinant proteins include: the GCN4 leucine zipper (Harbury et al. 1993 Science 262:1401-1407), the trimerization motif from the lung surfactant protein (Hoppe et al. 1994 FEBS Lett 344:191-195), collagen (McAlinden et al. 2003 J Biol Chem 278:42200-42207), and the phage T4 fibritin Foldon (Miroshnikov et al. 1998 Protein Eng 11:329-414), any of which can be linked to a coronavirus viral antigen or immunogen described herein (e.g., by linkage to the C-terminus of an S peptide) to promote trimerization of the recombinant viral antigen or immunogen. See also U.S. Pat. Nos. 7,268,116, 7,666,837, 7,691,815, 10,618,949, 10,906,944, and 10,960,070, and US 2020/0009244, which are incorporated herein by reference in their entireties for all purposes.

In some embodiments, one or more peptide linkers (such as a gly-ser linker, for example, a 10 amino acid glycine-serine peptide linker) can be used to link the recombinant viral antigen or immunogen to the multimerization domain. The trimer can include any of the stabilizing mutations provided herein (or combinations thereof) as long as the recombinant viral antigen or immunogen trimer retains the desired properties (e.g., the prefusion conformation).

To be therapeutically feasible, a desired trimerizing protein moiety for biologic drug designs should satisfy the following criteria. Ideally it should be part of a naturally secreted protein, like immunoglobulin Fc, that is also abundant (non-toxic) in the circulation, human in origin (lack of immunogenicity), relatively stable (long half-life) and capable of efficient self-trimerization which is strengthened by inter-chain covalent disulfide bonds so the trimerized coronavirus viral antigens or immunogens are structurally stable.

Collagen is a family of fibrous proteins that are the major components of the extracellular matrix. It is the most abundant protein in mammals, constituting nearly 25% of the total protein in the body. Collagen plays a major structural role in the formation of bone, tendon, skin, cornea, cartilage, blood vessels, and teeth. The fibrillar types of collagen I, II, III, IV, V, and XI are all synthesized as larger trimeric precursors, called procollagens, in which the central uninterrupted triple-helical domain consisting of hundreds of “G-X-Y” repeats (or glycine repeats) is flanked by non-collagenous domains (NC), the N-propeptide and the C-propeptide. Both the C- and N-terminal extensions are processed proteolytically upon secretion of the procollagen, an event that triggers the assembly of the mature protein into collagen fibrils which forms an insoluble cell matrix. BMP-1 is a protease that recognizes a specific peptide sequence of procollagen near the junction between the glycine repeats and the C-prodomain of collagens and is responsible for the removal of the propeptide. The shed trimeric C-propeptide of type I collagen is found in human sera of normal adults at a concentration in the range of 50-300 ng/mL, with children having a much higher level which is indicative of active bone formation. In people with familial high serum concentration of C-propeptide of type I collagen, the level could reach as high as 1-6 μg/mL with no apparent abnormality, suggesting the C-propeptide is not toxic. Structural study of the trimeric C-propeptide of collagen suggested that it is a tri-lobed structure with all three subunits coming together in a junction region near their N-termini to connect to the rest of the procollagen molecule. Such geometry in projecting proteins to be fused in one direction is similar to that of Fc dimer.

Type I, IV, V and XI collagens are mainly assembled into heterotrimeric forms consisting of either two α-1 chains and one α-2 chain (for Type I, IV, V), or three different a chains (for Type XI), which are highly homologous in sequence. The type II and III collagens are both homotrimers of α-1 chain. For type I collagen, the most abundant form of collagen, stable α(I) homotrimer is also formed and is present at variable levels in different tissues. Most of these collagen C-propeptide chains can self-assemble into homotrimers, when over-expressed alone in a cell. Although the N-propeptide domains are synthesized first, molecular assembly into trimeric collagen begins with the in-register association of the C-propeptides. It is believed the C-propeptide complex is stabilized by the formation of interchain disulfide bonds, but the necessity of disulfide bond formation for proper chain registration is not clear. The triple helix of the glycine repeats and is then propagated from the associated C-termini to the N-termini in a zipper-like manner. This knowledge has led to the creation of non-natural types of collagen matrix by swapping the C-propeptides of different collagen chains using recombinant DNA technology. Non-collagenous proteins, such as cytokines and growth factors, also have been fused to the N-termini of either pro-collagens or mature collagens to allow new collagen matrix formation, which is intended to allow slow release of the noncollagenous proteins from the cell matrix. However, under both circumstances, the C-propeptides are required to be cleaved before recombinant collagen fibril assembly into an insoluble cell matrix.

Although, other protein trimerization domains, such as those from GCN4 from yeast fibritin from bacteria phage T4 and aspartate transcarbamoylase of Escherichia coli, have been described previously to allow trimerization of heterologous proteins, none of these trimerizing proteins are human in nature, nor are they naturally secreted proteins. As such, any trimeric fusion proteins would have to be made intracellularly, which not only may fold incorrectly for naturally secreted proteins such as soluble receptors, but also make purification of such fusion proteins from thousands of other intracellular proteins difficult. Moreover, the fatal drawback of using such non-human protein trimerization domains (e.g. from yeast, bacteria phage and bacteria) for trimeric biologic drug design is their presumed immunogenicity in the human body, rendering such fusion proteins ineffective shortly after injecting them into the human body.

The use of collagen in a recombinant polypeptide as described herein thus has many advantages, including: (1) collagen is the most abundant protein secreted in the body of a mammal, constituting nearly 25% of the total proteins in the body; (2) the major forms of collagen naturally occur as trimeric helixes, with their globular C-propeptides being responsible for the initiating of trimerization; (3) the trimeric C-propeptide of collagen proteolytically released from the mature collagen is found naturally at sub microgram/mL level in the blood of mammals and is not known to be toxic to the body; (4) the linear triple helical region of collagen can be included as a linker with predicted 2.9 Å spacing per residue, or excluded as part of the fusion protein so the distance between a protein to be trimerized and the C-propeptide of collagen can be precisely adjusted to achieve an optimal biological activity; (5) the recognition site of BMP1 which cleaves the C-propeptide off the pro-collagen can be mutated or deleted to prevent the disruption of a trimeric fusion protein; (6) the C-propeptide domain self-trimerizes via disulfide bonds and it provides a universal affinity tag, which can be used for purification of any secreted fusion proteins created. In some embodiments, the C-propeptide of collagen to which the coronavirus viral antigen and immunogen, e.g., S protein peptide, enables the recombinant production of soluble, covalently-linked homotrimeric fusion proteins.

In some embodiments, the coronavirus viral antigen or immunogen is linked to a C-terminal propeptide of collagen to form a recombinant polypeptide. In some embodiments, the C-terminal propeptides of the recombinant polypeptides form inter-polypeptide disulfide bonds. In some embodiments, the recombinant proteins form trimers. In some embodiments, the coronavirus viral antigen or immunogen is an S protein peptide as described in Section I.

For example, a fusion polypeptide comprising a signal peptide MFVFLVLLPLVSS (SEQ ID NO: 54) on the N-terminus of the fusion polypeptide in SEQ ID NO: 1 may be produced and trimerized via inter-polypeptide disulfide bonds (Cys residues that may form inter-polypeptide disulfide bonds are bolded).

```
                10 20 30 40 50 60
MFVFLVLLPLVSSQCVNLTTRTQLPPAYTNSFTRGVYYPDKVFRSSVLHSTQDLFLPFFS
                70 80 90 100 110 120
NVTWHHALHVSGTNGTKRHDNPVLPHNDGVYFASTEKSNLLRGWIHGMTLDSKTQSLLIV
              130 140 150 160 170 180
NNATNVVIKVCEFQFCNDPFLGVYYHKNNKSWMESEFRVYSSANNCTFEYVSQPFLMDLE
              190 200 210 220 230 240
GKQGNFKNLREFVFKNIDGYFKIYSKHTPINLVRDLPQGFSALEPLVDLPIGINITRFQT
              250 260 270 280 290 300
LLALHRSYLTPGDSSSGWTAGAAAYYVGYLQPRTFLLKYNENGTITDAVDCALDPLSETK
              310 320 330 340 350 360
CTLKSFTVEKGIYQTSNFRVQPTESIVRFPNITNLCPFGEVFNATRFASVYAWNRKRISN
              370 380 390 400 410 420
CVADYSVLYNSASFSTFKCYGVSPTKLNDLCFTNVYADSFVIRGDEVRQIAPGQTGKIAD
              430 440 450 460 470 480
YNYKLPDDFTGCVIAWNSNNLDSKVGGNYNYLYRLFRKSNLKPFERDISTEIYQAGSTPC
              490 500 510 520 530 540
NGVEGFNCYFPLQSYGFQPTNGVGYQPYRVVVLSFELLHAPATVCGPKKSTNLVKNKCVN
              550 560 570 580 590 600
FNFNGLTGTGVLTESNKKFLPFQQFGRDIADTTDAVRDPQTLEILDITPCSFGGVSVITP
              610 620 630 640 650 660
GTNTSNQVAVLYQDVNCTEVPVAIHADQLTPTWRVYSTGSNVFQTRAGCLIGAEHVNNSY
              670 680 690 700 710 720
ECDIPIGAGICASYQTQTNSPRRARSVASQSIIAYTMSLGAENSVAYSNNSIAIPTNFTI
              730 740 750 760 770 780
SVTTEILPVSMTKTSVDCTMYICGDSTECSNLLLQYGSFCTQLNRALTGIAVEQDKNTQE
              790 800 810 820 830 840
VFAQVKQIYKTPPIKDFGGFNFSQILPDPSKPSKRSFIEDLLFNKVTLADAGFIKQYGDC
              850 860 870 880 890 900
LGDIAARDLICAQKFNGLTVLPPLLTDEMIAQYTSALLAGTITSGWTFGAGAALQIPFAM
              910 920 930 940 950 960
QMAYRFNGIGVTQNVLYENQKLIANQFNSAIGKIQDSLSSTASALGKLQDVVNQNAQALN
              970 980 990 1000 1010 1020
TLVKQLSSNFGAISSVLNDILSRLDKVEAEVQIDRLITGRLQSLQTYVTQQLIRAAEIRA
            1030 1040 1050 1060 1070 1080
SANLAATKMSECVLGQSKRVDFCGKGYHLMSFPQSAPHGVVFLHVTYVPAQEKNFTTAPA
            1090 1100 1110 1120 1130 1140
ICHDGKAHFPREGVFVSNGTHWFVTQRNFYEPQIITTDNTFVSGNCDVVIGIVNNTVYDP
            1150 1160 1170 1180 1190 1200
LQPELDSFKEELDKYFKNHTSPDVDLGDISGINASVVNIQKEIDRLNEVAKNLNESLIDL
            1210 1220 1230 1240 1250 1260
QELGKYEQYIKRSNGLPGPIGPPGPRGRTGDAGPVGPPGPPGPPGPPGPPSAGFDFSFLP
            1270 1280 1290 1300 1310 1320
QPPQEKAHDGGRYYRANDANVVRDRDLEVDTTLKSLSQQIENIRSPEGSRKNPARTCRDL
            1330 1340 1350 1360 1370 1380
KMCHSDWKSGEYWIDPNQGCNLDAIKVFCNMETGETCVYPTQPSVAQKNWYISKNPKDKR
            1390 1400 1410 1420 1430 1440
HVWFGESMTDGFQFEYGGQGSDPADVAIQLTFLRLMSTEASQNITYHCKNSVAYMDQQTG
            1450 1460 1470 1480 1490 1500
NLKKALLLQGSNEIEIRAEGNSRFTYSVTVDGCTSHTGAWGKTVIEYKTTKTSRLPIIDV
            1510 1520
APLDVGAPDQEFGFDVGPVCFL
```

In some embodiments, the inter-polypeptide disulfide bonds may comprise one or more or all of Cys15-136, Cys131-166, Cys291-301, Cys379-432, Cys336-361, Cys391-525, Cys480-488, Cys538-590, Cys617-649, Cys662-671, Cys743-749, Cys738-760, Cys840-851, Cys1032-1043, and Cys1082-1126, in any suitable combination. In some embodiments, the fusion polypeptide in the trimer may comprise one or more glycosylation sites (e.g., Asn-linked), for example, at one or more or all of Asn residues at 17, 61, 122, 149, 165, 234, 282, 331, 343, 603, 616, 657, 709, 717, 801, 1074, 1098, and 1134, in any suitable combination.

In some embodiments, the C-terminal propeptide is of human collagen. In some embodiments, the C-terminal propeptide comprises a C-terminal polypeptide of proα1(I), proα1(II), proα1(III), proα1(V), proα1(XI), proα2(I), proα2(V), proα2(XI), or proα3(XI), or a fragment thereof. In some embodiments, the C-terminal propeptide is or comprises a C-terminal polypeptide of proα1(I).

In some embodiments, the C-terminal propeptide is or comprises the amino acid sequence set forth in any of SEQ ID NOs: 67-80. In some embodiments, the C-terminal propeptide is an amino acid sequence having at least or about 85%, 90%, 92%, 95%, or 97% sequence identity to any of SEQ ID NOs: 67-80.

In some embodiments, the C-terminal propeptide is or comprises the amino acid sequence of a collagen trimerization domain (e.g., C-propeptide of human α1(I) collagen) with an aspartic acid (D) to asparagine (N) substitution in the BMP-1 site, for instance, as shown in SEQ ID NO: 68 where RAD is mutated to RAN. In some embodiments, the C-terminal propeptide is or comprises the amino acid sequence of a collagen trimerization domain (e.g., C-propeptide of human α1(I) collagen) with an alanine (A) to asparagine (N) substitution in the BMP-1 site, for instance, as shown in SEQ ID NO: 69 where RAD is mutated to RND. In some embodiments, the C-terminal propeptide herein may comprise a mutated BMP-1 site, e.g., RSAN instead of DDAN. In some embodiments, the C-terminal propeptide herein may comprise a BMP-1 site, e.g., a sequence (such as SEQ ID NO: 68 or 69) comprising the RAD (e.g., RADDAN) sequence instead of RAN (e.g., RANDAN) or RND (e.g., RNDDAN) may be used in a fusion polypeptide disclosed herein. For instance, SEQ ID NO: 27 (underlined) or a fragment, variant or mutant thereof may be directly or indirectly linked to SEQ ID NO: 67 (italicized) or a fragment, variant or mutant there, e.g., to form the following fusion protein:

QCVNLTTRTQLPPAYTNSFTRGVYYPDKVFRSSVLHSTQDLFLPFFSNVT
WFHAIHVSGTNGTKRFDNPVLPFNDGVYFASTEKSNIIRGWIFGTTLDSK
TQSLLIVNNATNVVIKVCEFQFCNDPFLGVYYHKNNKSWMESEFRVYSSA
NNCTFEYVSQPFLMDLEGKQGNFKNLREFVFKNIDGYFKIYSKHTPINLV
RDLPQGFSALEPLVDLPIGINITRFQTLLALHRSYLTPGDSSSGWTAGAA
AYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVA
DYSVLYNSASFSTFKCYGVSPTKLNDLCFTNVYADSFVIRGDEVRQIAPG
QTGKIADYNYKLPDDFTGCVIAWNSNNLDSKVGGNYNYLYRLFRKSNLKP
FERDISTEIYQAGSTPCNGVEGFNCYFPLQSYGFQPTNGVGYQPYRVVVL
SFELLHAPATVCGPKKSINLVKNKCVNFNFNGLTGTGVLTESNKKFLPFQ
QFGRDIADTTDAVRDPQTLEILDITPCSFGGVSVITPGINTSNQVAVLYQ
DVNCTEVPVAIHADQLTPTWRVYSTGSNVFQTRAGCLIGAEHVNNSYECD
IPIGAGICASYQTQTNSPRRARSVASQSIIAYTMSLGAENSVAYSNNSIA
IPTNFTISVTTEILPVSMTKTSVDCTMYICGDSTECSNLLLQYGSFCTQL
NRALTGIAVEQDKNTQEVFAQVKQIYKTPPIKDFGGFNFSQILPDPSKPS
KRSFIEDLLFNKVTLADAGFIKQYGDCLGDIAARDLICAQKFNGLTVLPP
LLTDEMIAQYTSALLAGTITSGWTFGAGAALQIPFAMQMAYRFNGIGVTQ
NVLYENQKLIANQFNSAIGKIQDSLSSTASALGKLQDVVNQNAQALNTLV
KQLSSNFGAISSVLNDILSRLDKVEAEVQIDRLITGRLQSLQTYVTQQLI
RAAEIRASANLAATKMSECVLGQSKRVDFCGKGYHLMSFPQSAPHGVVFL
HVTYVPAQEKNFTTAPAICHDGKAHFPREGVFVSNGTHWFVTORNFYEPQ
IITTDNTFVSGNCDVVIGIVNNTVYDPLQPELDSFKEELDKYFKNHTSPD
VDLGDISGINASVVNIQKEIDRLNEVAKNLNESLIDLQELGKYEQYIKRS
ANVVRDRDLEVDTTLKSLSQQIENIRSPEGSRKNPARTCRDLKMCHSDWK
SGEYWIDPNQGCNLDAIKVFCNMETGETCVYPTQPSVAQKNMYISKNPKD
KRHVWFGESMTDGFQFEYGGQGSDPADVAIQLTFLRLMSTEASQNITYHC
KNSVAYMDQQTGNLKKALLLQGSNEIEIRAEGNSRFTYSVTVDGCTSHTG
AWGKTVIEYKTTKTSRLPIIDVAPLDVGAPDQEFGFDVGPVCFL

In some embodiments, the C-terminal propeptide is or comprises an amino acid sequence that is a fragment of any of SEQ ID NOs: 67-80.

In some embodiments, the C-terminal propeptide can comprise a sequence comprising glycine-X-Y repeats, wherein X and Y are independently any amino acid, or an amino acid sequence at least 85%, 90°/%, 92%, 95%, or 97% identical thereto capable of forming inter-polypeptide disulfide bonds and trimerizing the recombinant polypeptides. In some embodiments, X and Y are independently proline or hydroxyproline.

In some cases where an S protein peptide is linked to the C-terminal propeptide to form the recombinant polypeptide, the recombinant polypeptides form a trimer resulting in a homotrimer of S protein peptides. In some embodiments, the S protein peptides of the trimerized recombinant polypetides are in a prefusion conformation. In some embodiments, the S protein peptides of the trimerized recombinant polypetides are in a postfusion conformation. In some embodiments, the confirmation state allows for access to different antigenic sites on the S protein peptides. In some embodiments, the antigenic sites are epitopes, such as linear epitopes or conformational epitopes. An advantage of having a trimerized recombinant polypeptides as described is that an immune response can be mounted against a variety of potential and diverse antigenic sites.

In some embodiments, trimerized recombinant polypeptides include individual recombinant polypeptides comprising the same viral antigen or immunogen. In some embodiments, trimerized recombinant polypeptides include individual recombinant polypeptides each comprising a different viral antigen or immunogen from the other recombinant polypeptides. In some embodiments, trimerized recombinant polypeptides include individual recombinant polypeptides wherein one of the individual recombinant polypeptides comprises a viral antigen or immunogen different from the other recombinant polypeptides. In some embodiments, trimerized recombinant polypeptides include individual recombinant polypeptides wherein two of the individual recombinant polypeptides comprise the same viral antigen or immunogen, and the viral antigen or immunogen is different from the viral antigen or immunogen comprised in the remaining recombinant polypeptide.

In some embodiments, the recombinant polypeptide comprises any coronavirus viral antigen or immunogen described in Section I. In some embodiments, the recombinant polypeptide comprises any coronavirus viral antigen or immunogen described in Section I linked, as described herein, to the C-terminal propeptide of collagen as described herein.

In some embodiments, the immunogen comprises a recombinant SARS-CoV or SARS-CoV-2 S ectodomain trimer comprising protomers comprising one or more (such as two, for example two consecutive) proline substitutions at or near the boundary between a HR1 domain and a central helix domain that stabilize the S ectodomain trimer in the prefusion conformation. In some such embodiments, the one or more (such as two, for example two consecutive) proline substitutions that stabilize the S ectodomain in the prefusion conformation are located between a position 15 amino acids N-terminal of a C-terminal residue of the HR1 and a position 5 amino acids C-terminal of a N-terminal residue of the central helix.

In some embodiments, the one or more (such as two, for example two consecutive) proline substitutions stabilize the coronavirus (e.g., SARS-CoV or SARS-CoV-2) S ectodomain trimer in the prefusion conformation. In some embodiments, the SARS-CoV-2 S protein peptide comprises 986K/987V to 986P/987P mutations.

In some embodiments, the recombinant coronavirus (e.g., SARS-CoV or SARS-CoV-2) S ectodomain trimer stabilized in the prefusion conformation comprises single-chain S ectodomain protomers comprising mutations to the S1/S2 and/or S2′ protease cleavage sites to prevent protease cleavage at these sites. In some embodiments, the SARS-CoV-2 S protein peptide comprises a 685R to 685A mutation. Exemplary protease cleavage sites for various viruses are shown below:

| Coronavirus | S1/S2, site 1 | S1/S2, site 2 | S2′ |
|---|---|---|---|
| 2019-nCoV | [symbol] | [symbol] | [symbol] |
| Cov-ZX21 | [symbol] | [symbol] | [symbol] |
| Bat-AC45 | [symbol] | [symbol] | [symbol] |
| SARS-CoV | [symbol] | [symbol] | [symbol] |
| BM48-31 | [symbol] | [symbol] | [symbol] |
| HXU9-1 | [symbol] | [symbol] | [symbol] |
| MERS-CoV | [symbol] | | [symbol] |
| HKU1 | [symbol] | | [symbol] |
| HCoV-OC43 | [symbol] | | [symbol] |
| UCoV-229E | [symbol] | | [symbol] |
| HCoV-NL63 | [symbol] | | [symbol] |

[symbol]

indicates data missing or illegible when filed

In some embodiments, the protomers of the recombinant coronavirus (e.g., SARS-CoV or SARS-CoV-2) S ectodomain trimer stabilized in the prefusion conformation by the one or more proline substitutions (such as 986P/987P substitutions) comprises additional modifications for stabilization in the prefusion conformation, such as a mutation at a protease cleavage site to prevent protease cleavage.

With reference to the SARS-CoV-2 S protein sequence provided as SEQ ID NO: 55, the ectodomain comprises a signal peptide (SP), which is removed during cellular processing; an N-terminal domain (NTD); a receptor binding domain (RBD); one or more S1/S2 cleavage sites; a fusion peptide (FP); internal fusion peptide (IFP); heptad repeat ½ (HR½), and the transmembrane domain (TM). Exemplary sources of the sequence can be found at ncbi.nlm.nih.gov/nuccore/MN908947.3, ncbi.nlm.nih.gov/nuccore/MN908947, ncbi.nlm.nih.gov/nuccore/MN908947.2. Additional sequences can be found at ncbi.nlm.nih.gov/genbank/sars-cov-2-seqs%, including the pneumonia virus isolate Wuhan-Hu-1, complete genome.

In some embodiments, the protomers of the prefusion-stabilized SARS-CoV-2 S ectodomain trimer can have a C-terminal residue (which can be linked to a trimerization domain, or a transmembrane domain, for example) of the C-terminal residue of the NTD, the RBD, S1 (at either the S1/S2 site 1, or S1/S2 site 2), FP, IFP, HR1, HR2, or the ectodomain. The position numbering of the S protein may vary between SARS-CoV stains, but the sequences can be aligned to determine relevant structural domains and cleavage sites. It will be appreciated that a few residues (such as up to 10) on the N and C-terminal ends of any of the ectodomain fragment can be removed or modified in the disclosed immunogens without decreasing the utility of the S ectodomain trimer as an immunogen.

In some embodiments, the recombinant polypeptide is or comprises an NTD peptide of SARS-CoV or SARS-CoV-2 S protein. In some embodiments, the recombinant polypeptide is or comprises an RBD peptide of SARS-CoV or SARS-CoV-2 S protein. In some embodiments, the recombinant polypeptide is or comprises an NTD peptide and an RBD peptide of SARS-CoV or SARS-CoV-2 S protein. In some embodiments, the recombinant polypeptide is or comprises an S1 domain peptide of SARS-CoV or SARS-CoV-2 S protein. In some embodiments, the recombinant polypeptide is or comprises an S2 domain peptide of SARS-CoV or SARS-CoV-2 S protein.

In some embodiments, the recombinant polypeptide or the fusion protein comprises a first sequence set forth in any of SEQ ID NOs: 27-66 linked to a second sequence set forth in any of SEQ ID NOs: 67-80, wherein the C terminus of the first sequence is directly or indirectly linked to the N terminus of the second sequence.

An exemplary SARS-CoV-1 S recombinant polypeptide without a signal peptide is provided in SEQ 1D NO: 26 (1491 aa):

```
                10 20 30 40 50 60
SDLDRCTTFD DVQAPNYTQH TSSMRGVYYP DEIFRSDTLY LTQDLFLPFY SNVTGFHTIN
                70 80 90 100 110 120
HTFDNPVIPF KDGIYFAATE KSNVVRGWVF GSTMNNKSQS VIIINNSTNV VIRACNFELC
              130 140 150 160 170 180
DNPFFAVSKP MGTQTHTMIF DNAFNCTFEY ISDAFSLDVS EKSGNFKHLP EFVFKNKDGF
              190 200 210 220 230 240
LYVYKGYQPI DVVRDLPSGF NTLKPIFKLP LGINITNFRA ILTAFLPAQD TWGTSAAAYF
              250 260 270 280 290 300
VGYLKPTTFM LKYDENGTIT DAVDCSQNPL AELKCSVKSF EIDKGIYQTS NFRVVPSRDV
              310 320 330 340 350 360
VRFPNITNLC PFGEVFNATK FPSVYAWERK RISNCVADYS VLYNSTFFST FKCYGVSAIK
              370 380 390 400 410 420
LNDLCFSNVY ADSFVVKGDD VRQIAPGQTG VIADYNYKLP DDFMGCVLAW NTRNIDATST
              430 440 450 460 470 480
GNYNYKYRYL RHGKLRPFER DISNVPFSPD GKPCTPPALN CYWPLNDYGF YTTTGIGYQP
              490 500 510 520 530 540
YRVVVLSFEL LNAPATVCGP KLSTDLIKNQ CVNFNFNGLT GTGVLTPSSK RFQPFQQFGR
              550 560 570 580 590 600
DVSDFTDSVR DPKTSEILDI SPCSFGGVSV ITPGTNASSE VAVLYQDVNC TDVSTAIHAD
              610 620 630 640 650 660
QLTPAWRIYS TGNNVFQTQA GCLIGAEHVD TSYECDIPIG AGICASYHTV SLLRSTSQKS
              670 680 690 700 710 720
IVAYTMSLGA DSSIAYSNNT IAIPTNFSIS ITTEVMPVSM AKTSVDCNMY ICGDSTECAN
              730 740 750 760 770 780
LLLQYGSFCT QLNRALSGIA AEQDRNTREV FAQVKQMYKT PTLKDFGGFN FSQILPDPLK
              790 800 810 820 830 840
PTKRSFIEDL LFNKVILADA GFMKQYGECL GDINARDLIC AQKFNGLTVL PPLLTDDMIA
              850 860 870 880 890 900
AYTAALVSGT ATAGWTFGAG AALQTPFAMQ MAYRFNGIGV TQNVLYENQK QIANQFNKAI
              910 920 930 940 950 960
SQIQESLTTT STALGKLQDV VNQNAQALNT LVKQLSSNFG AISSVLNDIL SRLDKVEAEV
              970 980 990 1000 1010 1020
QIDRLITGRL QSLQTYVTQQ LIRAAEIRAS ANLAATKMSE CVLGQSKRVD FCGKGYHLMS
            1030 1040 1050 1060 1070 1080
FPQAAPHGVV FLHVTYVPSQ ERNFTTAPAI CHEGKAYFPR EGVFVFNGTS WFITQRNFFS
            1090 1100 1110 1120 1130 1140
PQIITTDNTF VSGNCDVVIG IINNTVYDPL QPELDSFKEE LDKYFKNGTS PDVDLGDISG
            1150 1160 1170 1180 1190 1200
INASVVNIQE EIDRLNEVAK NLNESLIDLQ ELGKYEQYIK RSNGLPGPIG PPGPRGRIGD
            1210 1220 1230 1240 1250 1260
AGPVGPPGPP GPPGPPGPPS AGFDFSFLPQ PPQEKAHDGG RYYRANDANV VRDRDLEVDT
            1270 1280 1290 1300 1310 1320
TLKSLSQQIE NIRSPEGSRK NPARTCRDLK MCHSDQKSGE YWIDPNQGCN LDAIKVFCNM
            1330 1340 1350 1360 1370 1380
EIGETCVYPT QPSVAQKNWY ISKNPKDKRH VWFGESMTDG FQFEYGGQGS DPADVAIQLT
            1390 1400 1410 1420 1430 1440
FLRLMSTEAS QNITYHCKNS VAYMDQQTGN LKKALLLQGS NEIEIRAEGN SRFTYSVTVD
            1450 1460 1470 1480 1490
GCTSHTGAWG KTVIEYKTTK ISRLPIIDVA PLDVGAPDQE FGFDVGPVCF
```

The above SARS-CoV-1 S recombinant polypeptide may comprise an N-terminal signal peptide provided in SEQ 1D NO: 53.

An exemplary SARS-CoV-2 S recombinant polypeptide without a signal peptide is provided in SEQ ID NO: 1 (1509 aa):

```
                10 20 30 40 50 60
QCVNLTTRTQ LPPAYTNSFT RGVYYPDKVF RSSVLHSTQD LFLPFFSNVT WFHAIHVSGT
                70 80 90 100 110 120
NGTKRFDNPV LPFNDGVYFA STEKSNIIRG WIFGTTLDSK TQSLLIVNNA TNVVIKVCEE
              130 140 150 160 170 180
QFCNDPFLGV YYHKNNKSWM ESEFRVYSSA NNCTFEYVSQ PFLMDLEGKQ GNFKNLREFV
              190 200 210 220 230 240
FKNIDGYFKI YSKHTPINLV RDLPQGFSAL EPLVDLPIGI NITRFQTLLA LHRSYLTPGD
              250 260 270 280 290 300
SSSGWTAGAA AYYVGYLQPR TFLLKYNENG TITDAVDCAL DPLSETKCTL KSFTVEKGIY
              310 320 330 340 350 360
QTSNFRVQPT ESIVRFPNIT NLCPFGEVFN ATRFASVYAW NRKRISNCVA DYSVLYNSAS
              370 380 390 400 410 420
FSIFKUYGVS PTKLNDLCFT NVYADSFVIR GDEVRQIAPG QTGKIADYNY KLPDDFTGCV
              430 440 450 460 470 480
IAWNSNNLDS KVGGNYNYLY RLFRKSNLKP FERDISTEIY QAGSTPCNGV EGFNCYFPLQ
              490 500 510 520 530 540
SYGFQPTNGV GYQPYRVVVL SFELLHAPAT VCGPKKSTNL VKNKCVNFNF NGLTGIGVLT
              550 560 570 580 590 600
ESNKKFLPFQ QFGRDIADTT DAVRDPQTLE ILDITPCSFG GVSVITPGTN TSNQVAVLYQ
              610 620 630 640 650 660
DVNCTEVPVA IHADQLTPTW RVYSTGSNVF QTRAGCLIGA EHVNNSYECD IPIGAGICAS
              670 680 690 700 710 720
YQTQTNSPRR ARSVASQSII AYTMSLGAEN SVAYSNNSIA IPTNFTISVT TEILPVSMTK
              730 740 750 760 770 780
TSVDCTMYIC GDSTECSNLL LQYGSFCTQL NRALTGIAVE QDKNTQEVFA QVKQIYKTPP
              790 800 810 820 830 840
IKDFGGFNTS QILPDPSKPS KRSEIEDLLF NKVTLADAGF IKQYGDCLGD IAARDLICAQ
              850 860 870 880 890 900
KFNGLTVLPP LLTDEMIAQY TSALLAGTIT SGWTFGAGAA LQIPFAMQMA YRFNGIGVTQ
              910 920 930 940 950 960
NVLYENQKLI ANQFNSAIGK IQDSLSSTAS ALGKLQDVVN QNAQALNTLV KQLSSNFGA1
              970 980 990 1000 1010 1020
SSVLNDILSR LDKVEAEVQI DRLITGRLQS LQTYVTQQLI RAAEIRASAN LAATKMSECV
            1030 1040 1050 1060 1070 1080
LGQSKRVDFC GKGYHLMSFP QSAPHGVVFL HVTYVPAQEK NFTTAPAICH DGKAHFPREG
            1090 1100 1110 1120 1130 1140
VFVSNGTHWF VIQRNFYEPQ IITTDNTFVS GNCDVVIGIV NNTVYDPLQP ELDSFKEELD
            1150 1160 1170 1180 1190 1200
KYFKNHISPD VDLGDISGIN ASVVNIQKEI DRLNEVAKNL NESLIDLQEL GKYEQYIKRS
            1210 1220 1230 1240 1250 1260
NGLPGPIGPP GPRGRTGDAG PVGPPGPPGP PGPPGPPSAG FDFSFLPQPP QEKAHDGGRY
            1270 1280 1200 1300 1310 1320
YRANDANVVR DRDLEVDTTL KSLSQQIENI RSPEGSRKNP ARTCRDLKMC HSDWKSGEYW
            1330 1340 1350 1360 1370 1380
IDPNQGCNLD AIKVFCNMET GETCVYPTQP SVAQKNWYIS KNPKDKRHVW FGESMTDGFQ
            1390 1400 1410 1420 1430 1440
FEYGGQGSDP ADVAIQLTFL RLMSISASQN ITYHCKNSVA YMDQQTGNLK KALLLQGSNE
            1450 1460 1470 1480 1490 1500
IEIRAEGNSR FTYSVTVDGC TSHTGAWGKT V1EYKTTKTS RLPIIDVAPL DVGAPDQEFG
            1509
FDVGPVCFL
```

The above SARS-CoV-2 S recombinant polypeptide may comprise an N-terminal signal peptide provided in SEQ ID NO: 54.

In some embodiments, the recombinant polypeptide is or comprises the sequence set forth in SEQ ID NO: 1. In some embodiments, the recombinant polypeptide is or comprises an amino acid sequence having at least or about 80%, 81%, 82%, 83%, 84%, 85%, 86%, 87%, 88%, 89%, 90°/%, 91%, 92%, 93%, 94%, 95%, 96%, 97%, 98%, or 99% sequence identity to SEQ ID NO: 1, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 1 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 2. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 2, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 2 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, RI90S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 3. In some embodiments, the recombinant polypeptide is or comprises an amino acid sequence having at least or about 80%, 81%, 82%, 83%, 84%, 85%, 86%, 87%, 88%, 89%, 90°/%, 91%, 92%, 93%, 94%, 95%, 96%, 97%, 98%, or 99% sequence identity to SEQ ID NO: 3, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 3 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 4. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 4, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 4 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, RI90S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 5. In some embodiments, the recombinant polypeptide is or comprises an amino acid sequence having at least or about 80%, 81%, 82%, 83%, 84%, 85%, 86%, 87%, 88%, 89%, 90°/%, 91%, 92%, 93%, 94%, 95%, 96%, 97%, 98%, or 99% sequence identity to SEQ ID NO: 5, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 5 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 6. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99/% sequence identity to SEQ ID NO: 6, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 6 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, RI90S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 7. In some embodiments, the recombinant polypeptide is or comprises an amino acid sequence having at least or about 80%, 81%, 82%, 83%, 84%, 85%, 86%, 87%, 88%, 89%, 90°/%, 91%, 92%, 93%, 94%, 95%, 96%, 97%, 98%, or 99% sequence identity to SEQ ID NO: 7, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 7 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 8. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99/% sequence identity to SEQ ID NO: 8, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 8 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, RI90S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 9. In some embodiments, the recombinant polypeptide is or comprises an amino acid sequence having at least or about 80%, 81%, 82%, 83%, 84%, 85%, 86%, 87%, 88%, 89%, 90°/%, 91%, 92%, 93%, 94%, 95%, 96%, 97%, 98%, or 99% sequence identity to SEQ ID NO: 9, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 9 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 10. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 10, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 10 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 11. In some embodiments, the recombinant polypeptide is or comprises an amino acid sequence having at least or about 80%, 81%, 82%, 83%, 84%, 85%, 86%, 87%, 88%, 89%, 90°/%, 91%, 92%, 93%, 94%, 95%, 96%, 97%, 98%, or 99% sequence identity to SEQ ID NO: 11, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 11 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 12. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 12, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 12 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 13. In some embodiments, the recombinant polypeptide is or comprises an amino acid sequence having at least or about 80%, 81%, 82%, 83%, 84%, 85%, 86%, 87%, 88%, 89%, 90°/%, 91%, 92%, 93%, 94%, 95%, 96%, 97%, 98%, or 99% sequence identity to SEQ ID NO: 13, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 13 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 14. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 14, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 14 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 15. In some embodiments, the recombinant polypeptide is or comprises an amino acid sequence having at least or about 80%, 81%, 82%, 83%, 84%, 85%, 86%, 87%, 88%, 89%, 90°/%, 91%, 92%, 93%, 94%, 95%, 96%, 97%, 98%, or 99% sequence identity to SEQ ID NO: 15, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 15 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 16. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 16, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 16 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 17. In some embodiments, the recombinant polypeptide is or comprises an amino acid sequence having at least or about 80%, 81%, 82%, 83%, 84%, 85%, 86%, 87%, 88%, 89%, 90°/%, 91%, 92%, 93%, 94%, 95%, 96%, 97%, 98%, or 99% sequence identity to SEQ ID NO: 17, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 17 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 18. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 18, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 18 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 19. In some embodiments, the recombinant polypeptide is or comprises an amino acid sequence having at least or about 80%, 81%, 82%, 83%, 84%, 85%, 86%, 87%, 88%, 89%, 90°/%, 91%, 92%, 93%, 94%, 95%, 96%, 97%, 98%, or 99% sequence identity to SEQ ID NO: 19, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 19 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 20. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97⁰,%6, 98%, or 99% sequence identity to SEQ ID NO: 20, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 20 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 21. In some embodiments, the recombinant polypeptide is or comprises an amino acid sequence having at least or about 80%, 81%, 82%, 83%, 84%, 85%, 86%, 87%, 88%, 89%, 90°/%, 91%, 92%, 93%, 94%, 95%, 96%, 97%, 98%, or 99% sequence identity to SEQ ID NO: 21, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 21 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 22. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 22, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 22 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 23. In some embodiments, the recombinant polypeptide is or comprises an amino acid sequence having at least or about 80%, 81%, 82%, 83%, 84%, 85%, 86%, 87%, 88%, 89%, 90°/%, 91%, 92%, 93%, 94%, 95%, 96%, 97%, 98%, or 99% sequence identity to SEQ ID NO: 23, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 23 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 24. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 24, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 24 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 25. In some embodiments, the recombinant polypeptide is or comprises an amino acid sequence having at least or about 80%, 81%, 82%, 83%, 84%, 85%, 86%, 87%, 88%, 89%, 90°/%, 91%, 92%, 93%, 94%, 95%, 96%, 97%, 98%, or 99% sequence identity to SEQ ID NO: 25, including a sequence comprising substitution, deletion, and/or insertion at one or more amino acid positions, such as 13, 18, 20, 26, 69, 70, 80, 138, 142, 144, 152, 190, 215, 242, 243, 244, 246, 400, 401, 402, 417, 440, 452, 477, 484, 501, 570, 614, 655, 681, 682, 683, 684, 685, 701, 716, 888, 982, 1027, 1118, and/or 1176 (amino acid positions with respect to SEQ ID NO: 55), or any combination thereof. In some embodiments, the recombinant polypeptide is or comprises a variant of SEQ ID NO: 25 and the variant comprises any one, two, three, four, five or more of the mutations selected from the group consisting of S13I, L18F, T20N, P26S, Δ69-70 (ΔHV), D80A, D138Y, G142D, Δ144 (ΔY), W152C, R190S, D215G, Δ242-244 (ΔLAL), R246I, Δ400-402 (ΔFVI), K417T, K417N, N440K, L452R, S477N, S477G, E484K, E484Q, N501Y, A570D, D614G, H655Y, P681H, P681R, R682G, R683S, R685G, A701V, T716I, F888L, S982A, T1027I, D1118H, and V1176F, or any combination thereof.

In some embodiments, the recombinant polypeptide is or comprises the sequence set forth in SEQ ID NO: 26. In some embodiments, the recombinant polypeptide is or comprises an amino acid sequence having at least or about 80%, 81%, 82%, 83%, 84%, 85%, 86%, 87%, 88%, 89%, 90%, 91%, 92%, 93%, 94%, 95%, 96%, 97%, 98%, or 99% sequence identity to SEQ ID NO: 26, including a sequence comprising substitution, deletion, and/or insertion at one or more amino acid positions of SEQ ID NO: 26.

As indicated above, in some embodiments, the recombinant polypeptides provided herein associate not only to form trimers, but can also aggregate or be aggregated to generate proteins comprising a plurality of recombinant polypeptides. In some embodiments, the proteins formed have macrostructures. In some cases, the macrostructure may confer structural stability of the coronavirus viral antigen or immunogen recombinant polypeptides, which in turn can afford access to potentially antigenic sites capable of promoting an immune response.

In some embodiments, the trimerized recombinant polypeptides aggregate to form a protein containing a plurality of trimerized recombinant polypeptides. In some embodiments, the plurality of trimerized recombinant polypeptides forms a protein having a macrostructure.

In some embodiments, the proteins described herein comprising a plurality of recombinant polypeptides are an immunogen. In some embodiments, the proteins described herein comprising a plurality of recombinant polypeptides are comprised in a nanoparticle. For example, in some embodiments, the proteins are linked directly to a nanoparticle, e.g., protein nanoparticle. In some embodiments, the proteins are linked indirectly to a nanoparticle. In some embodiments, the proteins described herein comprising a plurality of recombinant polypeptides are comprised in virus-like particle (VLP).

In some embodiments, provided herein is a complex comprising a recombinant polypeptide selected from the group consisting of SEQ ID NOs: 1-26 or a fragment, variant, or mutant thereof, in any suitable combination. In some embodiments, provided herein is a complex comprising a trimer of a recombinant polypeptide selected from the group consisting of SEQ ID NOs: 1-26 or a fragment, variant, or mutant thereof, wherein the recombinant polypeptides are trimerized via inter-polypeptide disulfide bonds to form the trimer.

In some embodiments, provided herein is a fusion protein comprising a plurality of recombinant polypeptides, each recombinant polypeptide comprising, from amino to carboxy terminus: a) a first region comprising a portion of a coronavirus spike protein ectodomain that precedes a coronavirus spike protein receptor binding domain (RBD) as located in a nonchimeric coronavirus spike protein, of a first coronavirus; b) a second region comprising a coronavirus spike protein receptor binding domain (RBD) of a second coronavirus that is different from said first coronavirus; and c) a C-terminal propeptide of collagen, wherein the C-terminal propeptides of the recombinant polypeptides form inter-polypeptide disulfide bonds. In some embodiments, the fusion protein further comprises a third region between the second region and the C-terminal propeptide of collagen. In some embodiments, the third region comprises an S1 domain of a third coronavirus, wherein the third coronavirus is the same or different from the first coronavirus or second coronavirus. In some embodiments, the third region comprises an S2 domain of a fourth coronavirus, wherein the fourth coronavirus is the same or different from the first, second, or fourth coronavirus. In some embodiments, the first region comprises an N-terminal domain (NTD) of the first coronavirus. In some embodiments, the first region comprises one or more amino acid residues that is/are different from corresponding amino acid residue(s) in the second coronavirus. In some embodiments, the second region comprises one or more amino acid residues that is/are different from corresponding amino acid residue(s) in the first coronavirus. In some embodiments, the first and second coronaviruses are different variants or strains of the same coronavirus. In some embodiments, the first region comprises the NTD of the first coronavirus, the second region comprises the RBD of the second coronavirus, and the first and second coronaviruses are different variants of SARS-CoV-2. In some embodiments, the first coronavirus and the second coronavirus are independently selected from the group consisting of SARS-CoV-2 viruses of the B.1.526, B.1.1.143, P.2, B.1.351, P.1, B.1.1.7, B.1.617, and A.23.1 lineages.

In some embodiments, provided herein is a trimeric fusion protein comprising three recombinant polypeptides, each recombinant polypeptide comprising, from amino to carboxy terminus: a) a first region comprising a coronavirus spike protein N-terminal domain (NTD) of a SARS-CoV-2 of the B.1.526 lineage; b) a second region comprising a coronavirus spike protein receptor binding domain (RBD) of a SARS-CoV-2 of the B.1.351 lineage; and c) a C-terminal propeptide of collagen, wherein the C-terminal propeptides of the recombinant polypeptides form inter-polypeptide disulfide bonds.

In some embodiments, provided herein is a method for preventing infection by a coronavirus in a mammal, comprising immunizing a mammal with an effective amount of a fusion protein disclosed herein. In some embodiments, neutralizing antibodies against the first and the second coronaviruses are generated in the mammal. In some embodiments, the first and second coronaviruses are different variants of SARS-CoV-2, and neutralizing antibodies generated in the mammal neutralize two or more of SARS-CoV-2 viruses of the B.1.526, B.1.1.143, P.2, B.1.351, P.1, B.1.1.7. B.1.617, and A.23.1 lineages. In some embodiments, neutralizing antibodies generated in the mammal neutralize three or more of SARS-CoV-2 viruses of the B.1.526, B.1.1.143, P.2, B.1.351, P.1, B.1.1.7, B.1.617, and A.23.1 lineages. In some embodiments, the method comprises immunizing the mammal with two or more doses of the fusion protein. In some embodiments, the fusion protein is administered as a booster dose following one or more doses of an immunogen comprising a spike protein peptide comprising NTD and RBD from the same SARS-CoV-2 variant.

In some embodiments, provided herein are engineered fusion polypeptides that are derived or modified from the spike (S) glycoprotein of coronaviruses including SARS-CoV-1 and SARS-CoV-2. In some embodiments, compared to a wildtype S protein sequence of the coronavirus, the fusion polypeptides disclosed herein can be stabilized in a prefusion conformation. In some embodiments, fusion to the trimerization domain may prevent the S protein peptide in the fusion proteins from forming a straight helix (e.g., similar to what occurs during membrane fusion process). For instance, cryo-EM structures of an S-Trimer subunit vaccine candidate shows it predominantly adopts tightly closed pre-fusion state, unlike the full-length wild-type spike protein which forms both pre- and post-fusion states in the presence of detergent. Ma et al., J Virol (2021) doi:10.1128/JVI.00194-21. In some embodiments, the fusion proteins may comprise an altered soluble S sequence with modification(s) that inactivates the S1/S2 cleavage site; mutation(s) in the turn region between the heptad repeat 1 (HR1) region and the central helix (CH) region that prevents HR1 and CH to form a straight helix; and/or truncation of the heptad repeat 2 region (HR2) in addition to the stabilizing mutations. In some embodiments, the fusion proteins herein may but do not need to comprise one or more mutations such as K986GN987G, K986PN987P, K986GN987P or K986PN987G which are believed to stabilize the spike protein in a pre-fusion state. In some embodiments, mutations such as K986GN987G, K986PN987P, K986GN987P or K986PN987G are not necessary for stabilizing a fusion polypeptide disclosed herein comprising the Trimer-Tag@ trimerization domain.

In some of these embodiments, the mutation inactivating S1/S2 cleavage site can contain substitution of RRAR (682-685 in SEQ ID NO:55) with GSAG (SEQ ID NO: 60), and the mutation in the turn region can contain double mutation K986GN987G, K986PN987P, K986GN987P or K986PN987G. In some embodiments, truncation of HR2 entails deletion of one or more of the residues shown in SEQ ID NO: 65 at the C-terminus of the wildtype soluble S sequence. In some embodiments, the immunogen polypeptide can further include in the region of HR1 that interacts with HR2 (a) one or more proline or glycine substitutions, and/or (b) insertion of one or more amino acid residues. In some of these embodiments, the immunogen polypeptide can have one or more substitutions selected from A942P, S943P, A944P, A942G, S943G and A944G. In some of these embodiments, the insertion can be insertion of G or GS between any residues in A942-A944.

In some embodiments, a neutralizing immune response induced by the disclosed immunogens herein generates a neutralizing antibody against a coronavirus such as SARS-CoV-2. In some embodiments, the neutralizing antibody herein binds to a cellular receptor or coreceptor of a coronavirus such as SARS-CoV-2 or component thereof. In some embodiments, the viral receptor or coreceptor is a coronavirus receptor or coreceptor, preferably a pneumonia virus receptor or coreceptor, more preferably a human coronavirus receptor such as SARS-CoV-2 receptor or coreceptor. In some embodiments, the neutralizing antibody herein modulates, decreases, antagonizes, mitigates, blocks, inhibits, abrogates and/or interferes with at least one coronavirus such as SARS-CoV-2 activity or binding, or with a coronavirus such as SARS-CoV-2 receptor activity or binding, in vitro, in situ and/or in vivo, such as SARS-CoV-2 release, SARS-CoV-2 receptor signaling, membrane SARS-CoV-2 cleavage, SARS-CoV-2 activity, SARS-CoV-2 production and/or synthesis. In some embodiments, the disclosed immunogens herein induce neutralizing antibodies against SARS-CoV-2 that modulate, decrease, antagonize, mitigate, block, inhibit, abrogate and/or interfere with SARS-CoV-2 binding to a SARS-CoV-2 receptor or coreceptor, such as angiotensin converting enzyme 2 (ACE2), dipeptidyl peptidase 4 (DPP4), dendritic cell-specific intercellular adhesion molecule-3-grabbing non integrin (DC-SIGN), and/or liver/lymph node-SIGN (L-SIGN).

### III. Methods of Detection and Diagnosis

Lateral flow immunoassays are widely used in many different areas of analytical chemistry and medicine, for example, in clinical diagnosis to determine the presence of an analyte of interest in a sample, such as a bodily fluid. Previous lateral flow immunoassay work is exemplified by U.S. patents and patent application publications: U.S. Pat. Nos. 5,602,040; 5,622,871; 5,656,503; 6,187,598; 6,228,660; 6,818,455; 2001/0008774; 2005/0244986; U.S. Pat. No. 6,352,862; 2003/0207465; 2003/0143755; 2003/0219908; U.S. Pat. Nos. 5,714,389; 5,989,921; 6,485,982; Ser. No. 11/035,047; U.S. Pat. Nos. 5,656,448; 5,559,041; 5,252,496; 5,728,587; 6,027,943; 6,506,612; 6,541,277; 6,737,277 B1; 5,073,484; 5,654,162: 6,020,147; 4,956,302; 5,120,643; 6,534,320; 4,942,522; 4,703,017; 4,743,560; 5,591,645; and RE 38,430 E.

The test strips described herein are capable of detecting a functional attribute of an analyte, e.g., an interaction-blocking characteristic. In some embodiments, the analyte is a neutralizing (or blocking) antibody, e.g., an antibody that interrupts the interaction of two or more molecular components such as a viral protein and a cell-surface protein in a host. In some embodiments, the neutralizing antibody is an anti-coronavirus neutralizing antibody. In some embodiments, the neutralizing antibody is an anti-SARS-CoV-2 neutralizing antibody. In some embodiments, the neutralizing antibody is an anti-RBD neutralizing antibody, wherein the RBD is from a coronavirus, such as SARS-CoV-2 or SAR-CoV.

The devices described herein comprise a chromatographic strip comprising one or more test zones, and optionally one or more control zones. In some embodiments, the chromatographic strip is a membrane. In some embodiments, the chromatographic strip is a porous membrane. The pore size of the chromatographic strip may vary widely. In some embodiments, the chromatographic strip comprises pores of about 1 μm to about 20 μm, such any of about 1 μm to about 10 μm, about 5 μm to about 15 μm, or about 10 μm to about 20 μm. In some embodiments, the chromatographic strip comprises a bibulous material. In some embodiments, the chromatographic strip comprises a non-bibulous material. In some embodiments, the chromatographic strip comprises a material selected from the group consisting of a cellulose, cellulose blend, nitrocellulose, cellulose ester, mixed nitrocellulose ester, polyester, acrylonitrile copolymer, rayon, glass fiber, polyethylene terephthalate fibers, polypropylene, and combinations thereof. In some embodiments, the membrane is a nitrocellulose membrane.

In some embodiments, the chromatographic strip, or a portion thereof, is treated with a blocker, e.g., to increase specificity of any binding interactions. In some embodiments, the blocker comprises casein, bovine serum albumin (BSA), methylated BSA, whole animal serum, non-fat dry milk, or a combination thereof. When the chromatographic strip is blocked, the charge of a chromatographic strip, such as nitrocellulose, is neutralized and thus, no additional proteins or components thereof can bind to the blocked chromatographic strip. Additionally, the chromatographic structure of the chromatographic strip is altered and the flow may be more like a gliding or sliding flow instead of the flow of traditional chromatography. In some embodiments, the chromatographic strip supports.

Certain components of the test strips described herein comprise a detection agent to facilitate identification (qualitatively and/or quantitatively) of said components at certain zones of the test strips (e.g., a test zone, control zone). In some embodiments, the molecular component of a molecular binding system is a labeled with a detection agent. In some embodiments, the other component such as in the sample binding zone (e.g., an antibody or antigen binding fragment) is labeled with a detection agent. In some embodiments, wherein two or more component of a test strip are labeled with a detection agent, each component is labeled with a unique detection agent that can be differentiated from other detection agents of the test strip (e.g., based on color).

In some embodiments, the detection agent comprises an enzyme. In some embodiments, the detection agent comprises a polymeric enzyme comprising a plurality of enzymes. In some embodiments, the enzyme is selected from the group consisting of beta-D-galactosidase, glucose oxidase, horseradish peroxidase, alkaline phosphatase, beta-lactamase, glucose-6-phosphate dehydrogenase, urease, uricase, superoxide dismutase, luciferase, pyruvate kinase, lactate dehydrogenase, galactose oxidase, acetylcholine-sterase, enterokinase, tyrosinase, and xanthine oxidase.

In some embodiments, the detection agent comprises a detection particle. In some embodiments, the detection particle comprises an enzymatic particle (such as a nanoparticle), polystyrene particle (such as a microsphere), latex particle, particle comprising gold (such as a nano-gold particle), colloidal gold particle, metal particle (such as an iron oxide nanoparticle), magnetic particle, fluorescently detectable particle, or semi-conductor particle (such as a nanocrystal).

In some embodiments, the test strip further comprises an absorbent zone. Generally, the absorbent zone is configured, e.g., to remove excess fluid from the chromatographic strip in a reversible or non-reversible manner. In some embodiments, the absorbent zone is configured to be a reversible dessicant (allowing back flow of fluid from the absorbent zone). In some embodiments, the absorbent zone is configured to be a non-reversible dessicant. In some embodiments, the absorbent zone comprises a wicking pad. In some embodiments, the wicking pad comprises a bibulous material. In some embodiments, the wicking pad comprises a filter paper, glass fiber filter, or the like.

In some embodiments, the absorbent zone is located downstream of the chromatographic strip. In some embodiments, the absorbent zone is in capillary communication with the chromatographic strip.

In some embodiments, the test strip further comprising a sample addition zone comprising a sample pad. In some embodiments, the sample pad is in capillary communication with one or more downstream components of a test strip, e.g., the binding pad or chromatographic strip.

In some embodiments, the sample addition zone, including the sample pad, is configured to receive a sample. In some embodiments, the sample comprises a bodily fluid. In some embodiments, the sample is a whole blood sample. In some embodiments, the sample is a blood sample. In some embodiments, the sample is a body secretion sample. In some embodiments, the sample is a bronchial alveolar lavage fluid sample.

In some embodiments, disclosed herein is a method for analyzing a sample, comprising: contacting a sample with a protein comprising a plurality of recombinant polypeptides, each recombinant polypeptide comprising a surface antigen of a coronavirus linked to a C-terminal propeptide of collagen, wherein the C-terminal propeptides of the recombinant polypeptides form inter-polypeptide disulfide bonds, and wherein a binding between the protein and an analyte capable of specific binding to the surface antigen of the coronavirus is detected. In some embodiments, the analyte is an antibody, a receptor, or a cell recognizing the surface antigen, and the sample is a body fluid, including but not limited to sera or plasma, which contains the analyte.

In any of the preceding embodiments, the binding can indicate the presence of the analyte in the sample, and/or an infection by the coronavirus in a subject from which the sample is derived.

In any of the preceding embodiments, the method can be a lateral flow method or an ELISA. In any of the preceding embodiments, the protein can be labeled with colloidal gold particles and dried within a conjugate pad on a test strip. Also disclosed herein is a test strip comprising a chromatographic strip comprising a protein, wherein the protein comprises a plurality of recombinant polypeptides, each recombinant polypeptide comprising a surface antigen of a coronavirus linked to a C-terminal propeptide of collagen, wherein the C-terminal propeptides of the recombinant polypeptides form inter-polypeptide disulfide bonds. In some embodiments, the protein is labeled with colloidal gold particles and dried within a conjugate pad on the test strip.

In any of the preceding embodiments, a secondary antibody specific to the analyte can be immobilized within a test zone of a chromatographic membrane on a test strip. In any of the preceding embodiments, the secondary antibody can be an anti-IgG antibody or an anti-IgM antibody. In any of the preceding embodiments, the test strip can further comprise a control zone wherein an antibody specific to a C-terminal propeptide of collagen is immobilized. In any of the preceding embodiments, the test strip can further comprise a sample pad to which an analyte is loaded for analysis on one end of the test strip, and an absorbent pad on the opposite end which is in capillary communication with the sample pad. In some embodiments, the chromatographic strip further comprises a control zone, and wherein a control capture agent is immobilized within the control zone.

In any of the preceding embodiments, the test strip can further comprise a sample binding zone comprising a binding pad comprising the protein, and one end of the binding pad is in capillary communication with one end of the chromatographic strip.

In any of the preceding embodiments, the test strip can further comprise a sample addition zone comprising a sample pad, wherein the sample pad is in capillary communication with the binding pad or the chromatographic strip.

In any of the preceding embodiments, the analyte can comprise a neutralizing antibody against the surface antigen of the coronavirus.

In any of the preceding embodiments, the analyte can comprise a broad neutralizing antibody against the surface antigen of the coronavirus.

In any of the preceding embodiments, the analyte can comprise an IgG antibody.

In any of the preceding embodiments, the analyte can comprise an IgM antibody.

In any of the preceding embodiments, the analyte can comprise a human antibody.

In any of the preceding embodiments, the sample can be derived from a subject infected with the coronavirus.

In any of the preceding embodiments, the sample can be serum or plasma from a subject infected with the coronavirus and has recovered.

In any of the preceding embodiments, the sample can be derived from a subject immunized with a coronavirus vaccine.

In any of the preceding embodiments, a receptor for the surface antigen of an coronavirus, optionally the receptor is a receptor-Fc, such as ACE2-Fc, can be immobilized within a second test zone of a chromatographic membrane on a test strip.

In any of the preceding embodiments, a reduction in retention of antigen-labeled colloidal gold particles at the second test zone upon loading an analyte, compared to vehicle control without analyte, can indicate positive detection of neutralizing antibody or antibodies that is capable blocking the interaction between the receptor and the surface antigen of a coronavirus.

In any of the preceding embodiments, the coronavirus can be a Severe Acute Respiratory Syndrome (SARS)-coronavirus (SARS-CoV), a SARS-coronavirus 2 (SARS-CoV-2), a SARS-like coronavirus, a Middle East Respiratory Syndrome (MERS)-coronavirus (MERS-CoV), a MERS-like coronavirus, NL63-CoV, 229E-CoV, OC43-CoV, HKU1-CoV, WIV1-CoV, MHV, HKU9-CoV, PEDV-CoV, or SDCV.

In any of the preceding embodiments, the surface antigen can comprise a coronavirus spike (S) protein or a fragment or epitope thereof, wherein the epitope is optionally a linear epitope or a conformational epitope, and wherein the protein comprises three recombinant antigen polypeptides linked by C-terminal propeptide of collagen.

In any of the preceding embodiments, the surface antigen can comprise a signal peptide, an S1 subunit peptide, an S2 subunit peptide, or any combination thereof.

In any of the preceding embodiments, the surface antigen can comprise a signal peptide, a receptor binding domain (RBD) peptide, a receptor binding motif (RBM) peptide, a fusion peptide (FP), a heptad repeat 1 (HR1) peptide, or a heptad repeat 2 (HR2) peptide, or any combination thereof.

In any of the preceding embodiments, the surface antigen can comprise a receptor binding domain (RBD) of the S protein.

In any of the preceding embodiments, the surface antigen can comprise an S1 subunit and an S2 subunit of the S protein.

In any of the preceding embodiments, the surface antigen can lack a transmembrane (TM) domain peptide and/or a cytoplasm (CP) domain peptide.

In any of the preceding embodiments, the surface antigen can comprise a protease cleavage site, wherein the protease is optionally furin, trypsin, factor Xa, or cathepsin L.

In any of the preceding embodiments, the surface antigen can lack a protease cleavage site, wherein the protease is optionally furin, trypsin, factor Xa, or cathepsin L.

In any of the preceding embodiments, the surface antigen can be soluble or do not directly bind to a lipid bilayer, e.g., a membrane or viral envelope.

In any of the preceding embodiments, the surface antigen can be the same or different among the recombinant polypeptides of the protein.

In any of the preceding embodiments, the surface antigen can be directly fused to the C-terminal propeptide, or linked to the C-terminal propeptide via a linker, such as a linker comprising glycine-X-Y repeats, wherein X and Y and independently any amino acid and optionally proline or hydroxyproline.

In any of the preceding embodiments, the protein can bind to a cell surface receptor of a subject, optionally wherein the subject is a mammal such as a primate, e.g., human.

In any of the preceding embodiments, the cell surface receptor can be angiotensin converting enzyme 2 (ACE2), dipeptidyl peptidase 4 (DPP4), dendritic cell-specific intercellular adhesion molecule-3-grabbing non integrin (DC-SIGN), or liver/lymph node-SIGN (L-SIGN).

In any of the preceding embodiments, the C-terminal propeptide can be of human collagen.

In any of the preceding embodiments, the C-terminal propeptide can comprise a C-terminal polypeptide of proα1(I), proα1(II), proα1(III), proα1(V), proα1(XI), proα2(I), proα2(V), proα2(XI), or proα3(XI), or a fragment thereof.

In any of the preceding embodiments, the C-terminal propeptides can be the same or different among the recombinant polypeptides.

In any of the preceding embodiments, the C-terminal propeptide can comprise any of SEQ ID NOs: 67-80 or an amino acid sequence at least 90% identical thereto capable of forming inter-polypeptide disulfide bonds and trimerizing the recombinant polypeptides.

In any of the preceding embodiments, the C-terminal propeptide can comprise a sequence comprising glycine-X-Y repeats linked to the N-terminus of any of SEQ ID NOs: 67-80, wherein X and Y and independently any amino acid and optionally proline or hydroxyproline, or an amino acid sequence at least 90% identical thereto capable of forming inter-polypeptide disulfide bonds and trimerizing the recombinant polypeptides.

In any of the preceding embodiments, the surface antigen in each recombinant polypeptide can be in a prefusion conformation or a postfusion conformation.

In any of the preceding embodiments, the surface antigen in each recombinant polypeptide can comprise any of SEQ ID NOs: 27-66 or an amino acid sequence at least 80% identical thereto.

In any of the preceding embodiments, the recombinant polypeptide can comprise any of SEQ ID NOs: 1-26 or an amino acid sequence at least 80% identical thereto.

### IV. Articles of Manufacture or Kits

Also provided are articles of manufacture or kits containing the provided recombinant polypeptide, proteins, and immunogenic compositions. The articles of manufacture may include a container and a label or package insert on or associated with the container. Suitable containers include, for example, bottles, vials, syringes, test tubes, IV solution bags, etc. The containers may be formed from a variety of materials such as glass or plastic. In some embodiments, the container has a sterile access port. Exemplary containers include an intravenous solution bags, vials, including those with stoppers pierceable by a needle for injection. The article of manufacture or kit may further include a package insert indicating that the compositions can be used to treat a particular condition such as a condition described herein (e.g., coronavirus infection). Alternatively, or additionally, the article of manufacture or kit may further include another or the same container comprising a pharmaceutically-acceptable buffer. It may further include other materials such as other buffers, diluents, filters, needles, and/or syringes.

The label or package insert may indicate that the composition is used for treating an coronavirus infection in an individual. The label or a package insert, which is on or associated with the container, may indicate directions for reconstitution and/or use of the formulation. The label or package insert may further indicate that the formulation is useful or intended for subcutaneous, intravenous, or other modes of administration for treating or preventing a coronavirus infection in an individual.

The container in some embodiments holds a composition which is by itself or combined with another composition effective for treating, preventing and/or diagnosing the condition. The article of manufacture or kit may include (a) a first container with a composition contained therein (i.e., first medicament), wherein the composition includes the immunogenic composition or protein or recombinant polypeptide thereof: and (b) a second container with a composition contained therein (i.e., second medicament), wherein the composition includes a further agent, such as an adjuvant or otherwise therapeutic agent, and which article or kit further comprises instructions on the label or package insert for treating the subject with the second medicament, in an effective amount.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polypeptide” and “protein” are used interchangeably to refer to a polymer of amino acid residues, and are not limited to a minimum length. Polypeptides, including the provided receptors and other polypeptides, e.g., linkers or peptides, may include amino acid residues including natural and/or non-natural amino acid residues. The terms also include post-expression modifications of the polypeptide, for example, glycosylation, sialylation, acetylation, and phosphorylation. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

As used herein, a “subject” is a mammal, such as a human or other animal, and typically is human. In some embodiments, the subject, e.g., patient, to whom the agent or agents, cells, cell populations, or compositions are administered, is a mammal, typically a primate, such as a human. In some embodiments, the primate is a monkey or an ape. The subject can be male or female and can be any suitable age, including infant, juvenile, adolescent, adult, and geriatric subjects. In some embodiments, the subject is a non-primate mammal, such as a rodent.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. In some embodiments, sufficient or significant delay can, in effect, encompass prevention, in that the individual does not develop the disease. For example, a late stage cancer, such as development of metastasis, may be delayed.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, a composition refers to any mixture of two or more products, substances, or compounds, including cells. It may be a solution, a suspension, liquid, powder, a paste, aqueous, non-aqueous or any combination thereof.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

### EXAMPLES

The following examples are included for illustrative purposes only and are not intended to limit the scope of the invention.

### Example 1: Generation of Recombinant Polypeptides Comprising SARS-CoV-2 S Protein Peptides

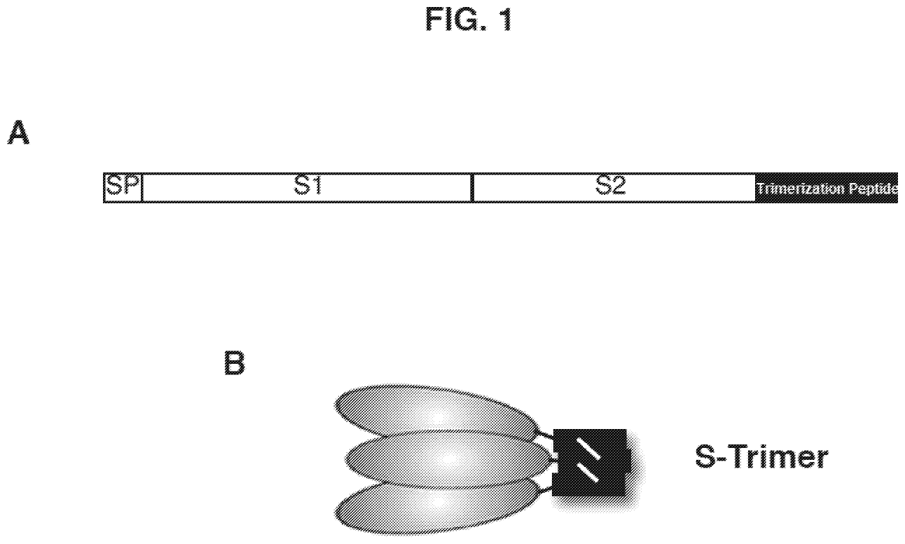

The complete ecto-domain of the native spike protein (S) from SARS-CoV2, including its signal peptide (SP), S1 and S2 domains, was fused in-frame at the C-terminus to a mammalian expression vector that encoded human C-propeptide of α1 collagen, to enable expression of a secreted and trimeric S-Trimer fusion antigen, e.g., as shown in FIG. 1.

High-level expression of S-Trimer fusion protein was achieved. An 8% SDS-PAGE analysis of S-Trimer expression from a fed-batch serum-free CHO cell culture in a 10 L bioreactor. 10 μL of cell-free conditioned medium from Day 6 to Day 11 were analyzed under reducing condition followed by Coomassie Blue staining. A highly purified S-Trimer was loaded on the gel as a reference standard (Std). The full-length S-Trimer and partially cleaved forms at S1/S2 furin site were as indicated.

Covalently linked S-Trimers were then purified and characterized. S-Trimer was purified from the cleared cell cultured medium via a Protein A (PA) affinity chromatography and anion exchange column (Q) followed by ultra-filtration and diafiltration (UF/DF) to obtain the drug substance (DS). Four μg of purified protein was analyzed against starting cell culture medium feed by an 8% reducing SDS-PAGE and stained with Coomassie Blue. The S-trimer was partially cleaved at the S1/S2 furin cleavage site, but the cleaved S1 subunit appeared to be bound to the S-Trimer since it was co-purified with the S-Trimer. The S-Trimer is a disulfide bond-linked trimer. Four μg of highly purified native-like S-Trimer was analyzed by a 6% SDS-PAGEs under non-reducing and reducing conditions as indicated and stained with Coomassie Blue. The S-Trimer was purified to nearly homogeneity as judged by SEC-HPLC analysis, with some cleaved S1 being separated during the size exclusion chromatography. The molecular weight of S-Trimer was estimated to be 660 Kda. The receptor binding kinetics of S-Trimer to ACE2-Fc was assessed by Fortebio biolayer interferometry measurements using a protein A sensor.

The S-Trimers were highly glycosylated with N-linked glycans. Highly purified S-Trimer before and after digestion with either endoglycanase F (PNGase F) alone or PNGase F plus endo-O-glycosidase to remove N- and O-linked glycans, and analyzed by an 8% reducing SDS-PAGE and stained with Coomassie Blue, to show the full-length S-Trimer, S2-Trimer and cleaved S1 before and after deglycosylation. Highly purified S-Trimers were visualized by negative EM using FEI Tecnai spirit electron microscopy.

### Example 2: Methods of Detecting Analytes Using Recombinant Polypeptides Comprising SARS-CoV-2 S Protein Peptides

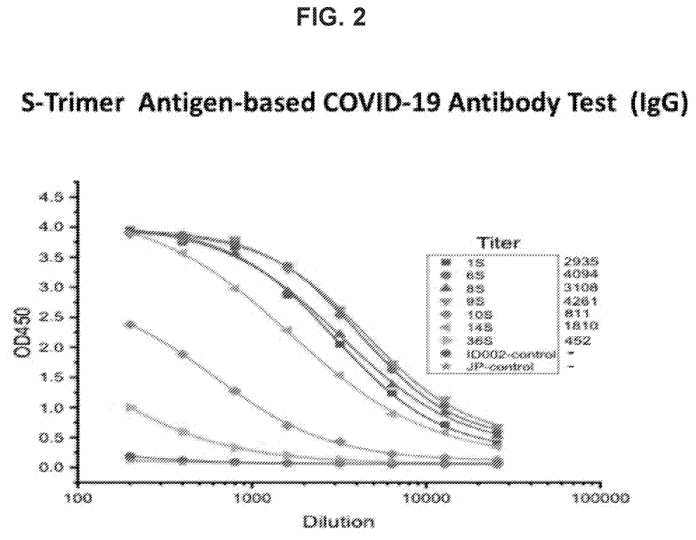

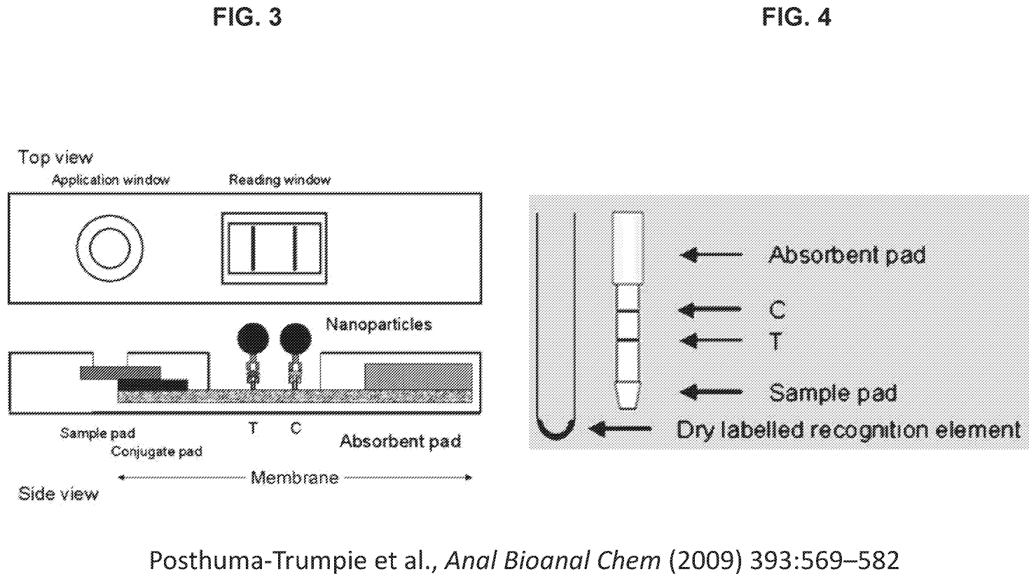

An ELISA was designed to provide a S-Trimer antigen-based SARS-CoV-2 antibody test, using the exemplary recombinant polypeptides generated as described in Example 1. Specifically, a plate was coated with recombinant S-Trimer in order to detect IgG antibodies in patient and normal control sera that recognize the S protein. Detection was done by goat anti-human IgG-HRP, and antibody titers were calculated as EC50 based on sample dilutions. FIG. 2 shows results of the ELISA assay, which demonstrate that S-Trimer was able to specifically detect S-reactive IgG antibodies in COVID-19 patient sera.

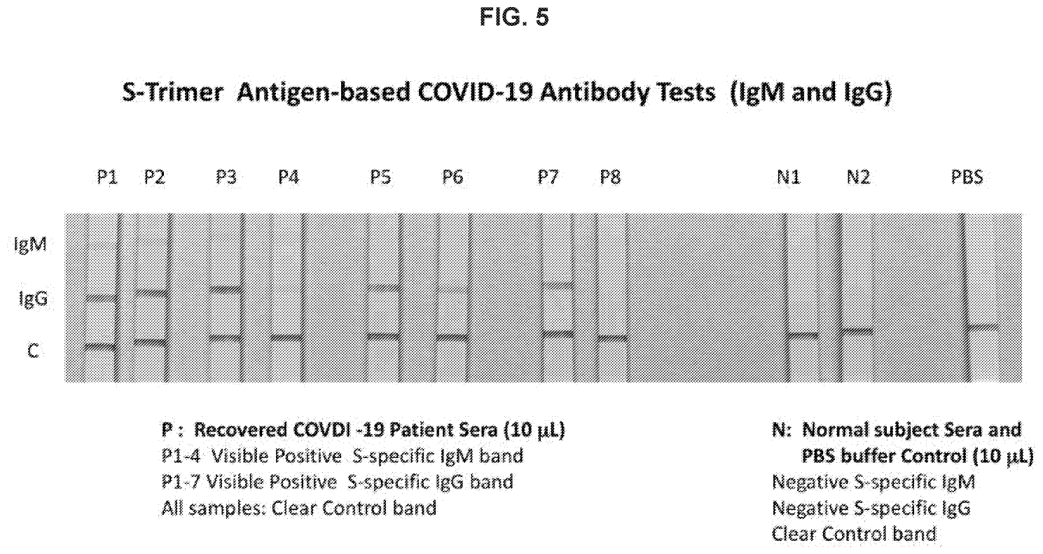

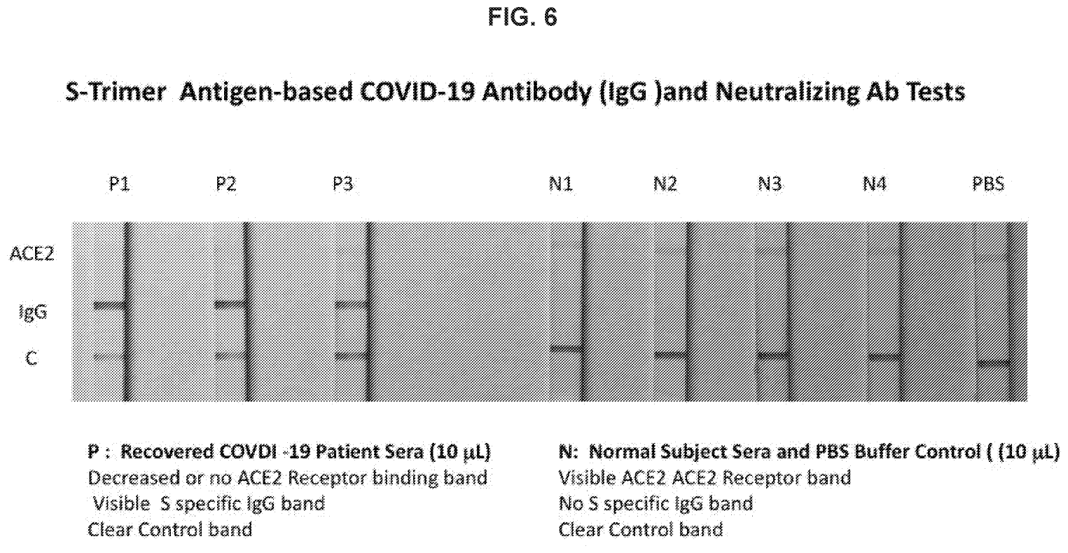

Sera from multiple patients who had recently recovered from COVID-19 were also analyzed with S-Trimer using lateral flow assays (FIG. 5 and FIG. 6). In the S-Trimer antigen-based SARS-CoV-2 antibody test for IgM and IgG, four out of the eight patient samples showed visible positive signals for S-specific IgM (FIG. 5, P1-P4), while seven out of eight showed visible positive signals for S-specific IgG (FIG. 5, P1-P7).

In the S-Trimer antigen-based SARS-CoV-2 antibody IgG and neutralizing antibody test, three out of the three patient samples showed visible positive signals for S-specific IgG, as well as decreased or no ACE2 binding band (FIG. 6, P1-P3). In all of the normal samples and PBS control, there were visible bands for ACE2 binding and no S-specific IgG binding (FIG. 6, N1-N4 and PBS). The S-Trimer was labeled with colloidal gold particles and dried within a conjugate pad on a test strip. A secondary antibody specific to the analyte (e.g., an anti-IgG antibody recognizing S-reactive IgG antibodies) was immobilized within a test zone of a chromatographic membrane on the test strip. In addition, a receptor for the S protein, such as ACE2-Fc, was immobilized within a second test zone of the chromatographic membrane on the test strip. These results collectively show that S-Trimer was able to specifically detect not only S-reactive IgG antibodies in COVID-19 patient sera, but also neutralizing antibodies in patient sera that were able to disrupt or reduce binding of S protein to its cell surface receptor ACE2.

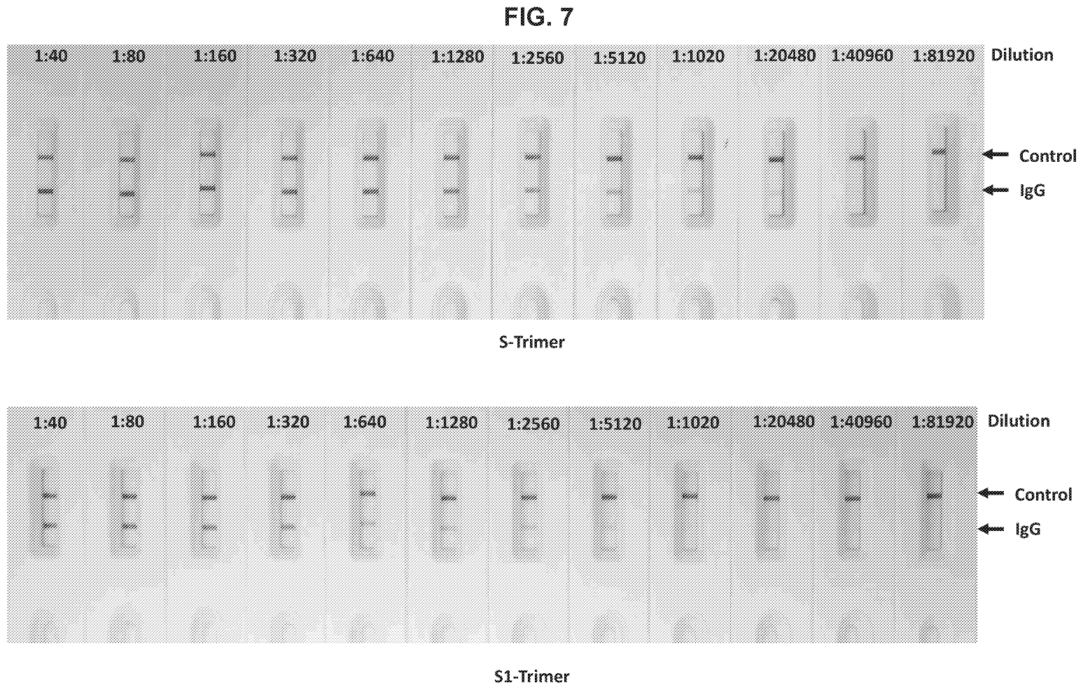

A convalescent serum sample was serially diluted and analyzed with an S-Trimer (FIG. 7, upper panel) and with an S1-Trimer (FIG. 7, lower panel) as the antigen using lateral flow assay. Visible positive signals for S-specific IgG were detected at 1:20480 to 1:40960 serial dilutions, whereas visible positive signals for S1-specific IgG were detected at 1:1020 to 1:20480 serial dilutions. These results show that the S-Trimer and S1-Trimer based assays are extremely sensitive.

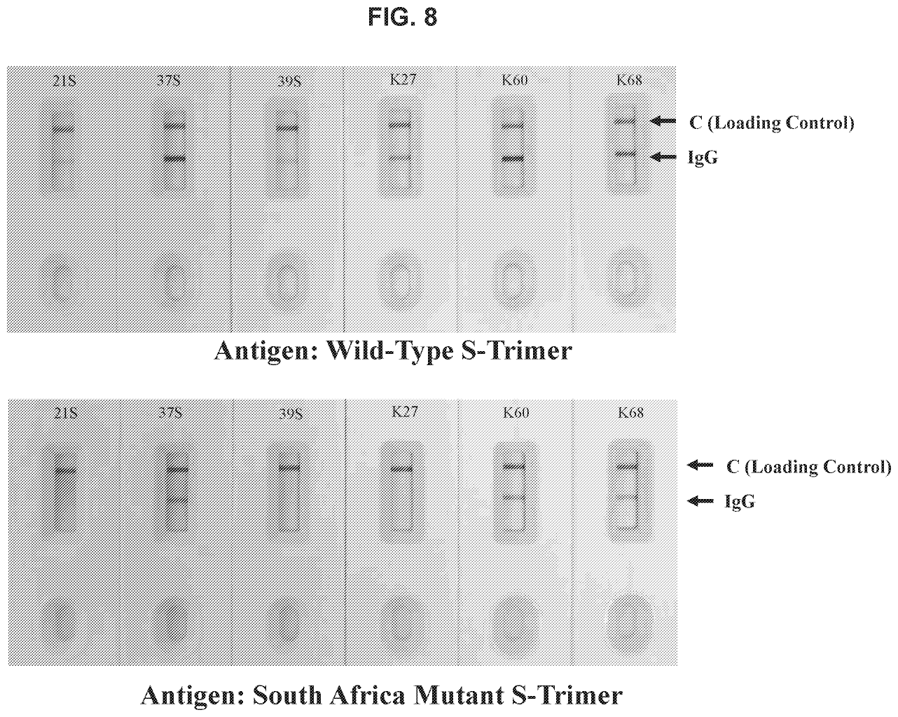

Multiple samples of convalescent sera were tested using lateral flow assays for S-reactive antibodies using wildtype S-Trimer (prototypic SARS-CoV-2 S-Trimer) and a B.1.351 South African variant SARS-CoV-2 S-Trimer (FIG. 8). Visible positive signals for S-specific IgG antibodies were observed in multiple samples using either wildtype S-Trimer or B.1.351 S-Trimer.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

SEQUENCES

| SEQ ID NO. | SEQUENCE | DESCRIPTION |
|---|---|---|
| 1 | QCVNLTTRTQLPPAYTNSFTRGVYYPDKVFRSSVLHSTQDLFLPFFSNVTWFHAIHVSGI | Prototypic |
| | NGTKRFDNPVLPFNDGVYFASTEKSNIIRGWIFGTTLDSKTQSLLIVNNATNVVIKVCEF | SARS-CoV-2 |
| | QFCNDPFLGVYYHKNNKSWMESEFRVYSSANNCTFEYVSQPFLMDLEGKQGNFKNLREFV | spike S- |
| | FKNIDGYFKIYSKHTPINLVRDLPQGFSALEPLVDLPIGINITRFQTLLALHRSYLTPGD | Trimer |
| | SSSGWTAGAAAYYVGYLQPRTFLLKYNENGTITDAVDCALDPLSETKCTLKSFTVEKGIY | polypeptide |
| | QTSNFRVQPTESIVRFPNITNLCPFGEVFNATRFASVYAWNRKRISNCVADYSVLYNSAS | without |
| | FSTFKCYGVSPTKLNDLCFTNVYADSFVIRGDEVRQIAPGQTGKIADYNYKLPDDFTGCV | signal |
| | IAWNSNNLDSKVGGNYNYLYRLFRKSNLKPFERDISTEIYQAGSIPCNGVEGFNCYFPLQ | peptide, |
| | SYGFQPTNGVGYQPYRVVVLSFELLHAPATVCGPKKSTNLVKNKCVNFNFNGLTGTGVLI | 1509 aa |
| | ESNKKFLPFQQFGRDIADTTDAVRDPQTLEILDITPCSFGGVSVITPGTNTSNQVAVLYQ | |
| | DVNCTEVPVAIHADQLTPTWRVYSTGSNVFQTRAGCLIGAEHVNNSYECDIPIGAGICAS | |
| | YQTQTNSPRRARSVASQSIIAYTMSLGAENSVAYSNNSIAIPTNFTISVTTEILPVSMTK | |
| | TSVDCTMYICGDSTECSNLLLQYGSFCTQLNRALTGIAVEQDKNTQEVFAQVKQIYKTPP | |
| | IKDFGGFNFSQILPDPSKPSKRSFIEDLLFNKVTLADAGFIKQYGDCLGDISARDLICAQ | |
| | KFNGLTVLPPLLTDEMIAQYTSALLAGTITSGWTFGAGAALQIPFAMQMAYRFNGIGVTQ | |
| | NVLYENQKLIANQFNSAIGKIQDSLSSTASALGKLQDVVNQNAQALNTLVKQLSSNFGAI | |
| | SSVLNDILSRLDKVEAEVQIDRLITGRLQSLQTYVTQQLIRAAEIRASANLAATKMSECV | |
| | LGQSKRVDFCGKGYHLMSFPQSAPHGVVFLHVTYVPAQEKNFTTAPAICHDGKAHFPREG | |
| | VFVSNGTHWFVTQRNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQYIKRS | |
| | NGLPGPIGPPGPRGRTGDAGPVGPPGPPGPPGPPGPPSAGFDFSFLPQPPQEKAHDGGRY | |
| | YRANDANVVRDRDLEVDTTLKSLSQQIENIRSPEGSRKNPARTCRDLKMCHSDWKSGEYW | |
| | IDPNQGCNLDAIKVFCNMETGETCVYPTOPSVAQKNWYISKNPKDKRHVWFGESMTDGFQ | |
| | FEYGGQGSDPADVAIQLTFLRLMSTEASQNITYHCKNSVAYMDQQTGNLKKALLLQGSNE | |
| | IEIRAEGNSRFTYSVTVDGCTSHTGAWGKTVIEYKTTKTSRLPIIDVAPLDVGAPDQEFG | |
| | FDVGPVCFL | |
| 2 | QCVNLTTRTQLPPAYTNSFTRGVYYPDKVFRSSVLHSTQDLFLPFFSNVTWFHAIHVSGT | Prototypic |
| | NGTKRFDNPVLPFNDGVYFASTEKSNIIRGWIFGTTLDSKTQSLLIVNNATNVVIKVCEF | SARS-CoV-2 |
| | QFCNDPFLGVYYHKNNKSWMESEFRVYSSANNCTFEYVSQPFLMDLEGKQGNFKNLREFV | spike S- |
| | FKNIDGYFKIYSKHTPINLVRDLPQGFSALEPLVDLPIGINITRFQTLLALHRSYLTPGD | Trimer |
| | SSSGWTAGAAAYYVGYLQPRTFLLKYNENGTITDAVDCALDPLSETKCTLKSFTVEKGIY | fusion |
| | QTSNFRVQPTESIVRFPNITNLCPFGEVFNATRFASVYAWNRKRISNCVADYSVLYNSAS | polypeptide |
| | FSTFKCYGVSPTKLNDLCFINVYADSFVIRGDEVRQIAPGQTGKIADYNYKLPDDFTGCV | without |
| | IAWNSNNLDSKVGGNYNYLYRLFRKSNLKPFERDISTEIYQAGSTPCNGVEGFNCYFPLQ | signal |
| | SYGFQPTNGVGYQPYRVVVLSFELLHAPATVCGPKKSTNLVKNKCVNFNFNGLTGTGVLT | peptide, |
| | ESNKKFLPFQQFGRDIADTTDAVRDPQTLEILDITPCSFGGVSVITPGTNTSNQVAVLYQ | 1509 aa, |
| | DVNCTEVPVAIHADQLTPTWRVYSTGSNVFQTRAGCLIGAEHVNNSYECDIPIGAGICAS | S1/S2 furin |
| | YQTQTNSPRRAASVASQSIIAYTMSLGAENSVAYSNNSIAIPTNFTISVTTEILPVSMTK | cleavage |
| | TSVDCTMYICGDSTECSNLLLQYGSFCTQLNRALTGIAVEQDKNTQEVFAQVKQIYKTPP | site 1 |
| | IKDFGGFNFSQILPDPSKPSKRSFIEDLLFNKVTLADAGFIKQYGDCLGDIAARDLICAQ | mutant |
| | KFNGLTVLPPLLIDEMIAQYTSALLAGIITSGWTFGAGAALQIPFAMQMAYRFNGIGVTQ | (685R→685A) |
| | NVLYENQKLIANQFNSAIGKIQDSLSSTASALGKLQDVVNQNAQALNTLVKOLSSNFGAI | |
| | SSVLNDILSRLDKVEAEVQIDRLITGRLQSLQTYVTQQLIRAAEIRASANLAATKMSECV | |
| | LGQSKRVDFCGKGYHLMSFPQSAPHGVVFLHVTYVPAQEKNFTTAPAICHDGKAHFPREG | |
| | VFVSNGTHWFVTQRNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQYIKRS | |
| | NGLPGPIGPPGPRGRTGDAGPVGPPGPPGPPGPPGPPSAGFDFSFLPQPPQEKAHDGGRY | |
| | YRANDANVVRDRDLEVDTTLKSLSQQIENIRSPEGSRKNPARTCRDLKMCHSDWKSGEYW | |
| | IDPNQGCNLDAIKVFCNMETGETCVYPTQPSVAQKNWYISKNPKDKRHVWFGESMTDGFQ | |
| | FEYGGQGSDPADVAIQLTFLRLMSTEASQNITYHCKNSVAYMDQQTGNLKKALLLQGSNE | |
| | IEIRAEGNSRFTYSVTVDGCTSHTGAWGKTVIEYKTTKTSRLPIIDVAPLDVGAPDQEFG | |
| | FDVGPVCFL | |
| 3 | QCVNLTTRTQLPPAYTNSFTRGVYYPDKVFRSSVLHSTQDLFLPFFSNVTWFHAIHVSGT | Prototypic |
| | NGTKRFDNPVLPFNDGVYFASTEKSNIIRGWIFGTTLDSKTQSLLIVNNATNVVIKVCEF | SARS-CoV-2 |
| | QFCNDPFLGVYYHKNNKSWMESEFRVYSSANNCTFEYVSQPFLMDLEGKQGNFKNLREFV | spike S- |
| | FKNIDGYFKIYSKHTPINLVRDLPQGFSALEPLVDLPIGINITRFQTLLALHRSYLTPGD | Trimer |
| | SSSGWTAGAAAYYVGYLQPRIFLLKYNENGTITDAVDCALDPLSETKCTLKSFTVEKGIY | fusion |
| | QTSNFRVQPTESIVRFPNITNLCPFGEVFNATRFASVYAWNRKRISNCVADYSVLYNSAS | polypeptide |
| | FSTFKCYGVSPTKLNDLCFTNVYADSFVIRGDEVRQIAPGQTGKIADYNYKLPDDFTGCV | without |
| | IAWNSNNLDSKVGGNYNYLYRLFRKSNLKPFERDISTEIYQAGSTPCNGVEGFNCYFPLQ | signal |
| | SYGFQPTNGVGYQPYRVVVLSFELLHAPATVCGPKKSTNLVKNKCVNFNFNGLTGTGVLI | peptide, |
| | ESNKKFLPFQQFGRDIADTTDAVRDPQTLEILDITPCSFGGVSVITPGTNTSNQVAVLYQ | 1509 aa, |
| | DVNCTEVPVAIHADQLTPTWRVYSTGSNVFQTRAGCLIGAEHVNNSYECDIPIGAGICAS | proline |
| | YQTQTNSPRRARSVASQSIIAYTMSLGAENSVAYSNNSIAIPTNFTISVTTEILPVSMTK | mutant |
| | TSVDCTMYICGDSTECSNLLLQYGSFCTQLNRALTGIAVEQDKNTQEVFAQVKQIYKIPP | (986K/987V→ |
| | IKDFGGFNFSQILPDPSKPSKRSFIEDLLFNKVTLADAGFIKQYGDCLGDIAARDLICAQ | 986P/987P) |
| | KFNGLTVLPPLLTDEMIAQYTSALLAGTITSGWTFGAGAALQIPFAMQMAYRFNGIGVTQ | |
| | NVLYENQKLIANQFNSAIGKIQDSLSSTASALGKLQDVVNQNAQALNTLVKQLSSNFGAI | |
| | SSVLKDILSRLDPPEAEVQIDRLITGRLQSLQTYVTQQLIRAAEIRASANLAATKMSECV | |
| | LGQSKRVDFCGKGYHLMSFPQSAPHGVVFLHVTYVPAQEKNFTTAPAICHDGKAHFPREG | |
| | VFVSNGTHWFVTORNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQYIKRS | |
| | NGLPGPIGPPGPRGRTGDAGPVGPPGPPGPPGPPGPPSAGFDFSFLPQPPQEKAHDGGRY | |
| | YRANDANVVRDRDLEVDTTLKSLSQQIENIRSPEGSRKNPARTCRDLKMCHSDWKSGEYW | |
| | IDPNQGCNLDAIKVFCNMETGETCVYPTQPSVAQKNWYISKNPKDKRHVWFGESMTDGFQ | |
| | FEYGGQGSDPADVAIQLTFLRLMSTEASQNITYACKNSVAYMDQQTGNLKKALLLQGSNE | |
| | IEIRAEGNSRFTYSVTVDGCTSHIGAWGKTVIEYKTTKTSRLPIIDVAPLDVGAPDQEFG | |
| | FDVGPVCFL | |
| 4 | QCVNLTTRTQLPPAYTNSFTRGVYYPDKVFRSSVLHSTQDLFLPFFSNVTWFHAIHVSGT | Prototypic |
| | NGTKRFDNPVLPFNDGVYFASTEKSNIIRGWIFGTTLDSKTQSLLIVNNATNVVIKVCEF | SARS-CoV-2 |
| | QFCNDPFLGVYYHKNNKSWMESEFRVYSSANNCTFEYVSQPFLMDLEGKQGNFKNLREFV | spike S- |
| | FKNIDGYFKIYSKHTPINLVRDLPQGFSALEPLVDLPIGINITRFQTLLALHRSYLTPGD | Trimer |
| | SSSGWTAGAAAYYVGYLQPRTFLLKYNENGTITDAVDCALDPLSETKCTLKSFTVEKGIY | fusion |
| | QTSNFRVOPTESIVRFPNITNLCPFGEVFNATRFASVYAWNRKRISNCVADYSVLYNSAS | polypeptide |
| | FSTFKCYGVSPTKLNDLCFTNVYADSFVIRGDEVRQIAPGQTGKIADYNYKLPDDFTGCV | without |
| | IAWNSNNLDSKVGGNYNYLYRLFRKSNLKPFERDISTEIYQAGSTPCNGVEGFNCYFPLQ | signal |
| | SYGFQPTNGVGYOPYRVVVLSFELLHAPATVCGPKKSTNLVKNKCVNFNFNGLIGTGVLT | peptide, |
| | ESNKKFLPFQQFGRDIADTTDAVRDPQTLEILDITPCSFGGVSVITPGTNTSNQVAVLYQ | 1509 aa, |
| | DVNCTEVPVAIHADQLTPTWRVYSTGSNVFQTRAGCLIGAEHVNNSYECDIPIGAGICAS | S1/S2 furin |
| | YQTQTNSPRRAASVASQSIIAYTMSLGAENSVAYSNNSIAIPTNFTISVTTEILPVSMTK | cleavage |
| | TSVDCTMYICGDSTECSNLLLQYGSFCTQLNRALTGIAVEQDKNTQEVFAQVKQIYKTPP | site 1 and |
| | IKDFGGFNFSQILPDPSKPSKRSFIEDLLFNKVTLADAGIIKQYGDCLGDIAARDLICAQ | proline |
| | KFNGLTVLPPLLTDEMIAQYTSALLAGTITSGWTFGAGAALQIPFAMQMAYRFNGIGVTQ | mutant |
| | NVLYENQKLIANQFNSAIGKIQDSLSSTASALGKLQDVVNQNAQALNTLVKQLSSNFGAI | (685R→685A, |
| | SSVLNDILSRLDPPEAEVQIDRL1TGRLQSLQTYVTQQLIRAAEIRASANLAAIKMSECV | 986K/987V→ |
| | LGQSKRVDFCGKGYHLMSFPQSAPHGVVFLHVTYVPAQEKNFTTAPAICRDGKAHFPREG | 986P/987P) |
| | VFVSNGTHWFVTQRNFYEPQIITTDNTFVSGNCDVVIGIVNNTVYDPLQPELDSFKEELD | |
| | KYFKNHTSPDVDLGDISGiNASVVNTQKETDRLNEVAKNLNESLlDLQELGKYEQYIKRS | |
| | NGLPGPIGPPGPRGRIGDAGPVGPPGPPGPPGPPGPPSAGFDFSFLPQPPQEKAHDGGRY | |
| | YRANDANVVRDRDLEVDTTLKSLSQQIENIRSPEGSRKNPARTCRDLKMCHSDWKSGEYW | |
| | IDPNQGCNLDAIKVFCNMETGETCVYPTQPSVAQKNWYISKNPKDKRHVWFGESMTDGFQ | |
| | FEYGGQGSDPADVAIQLIFLRLMSTEASQNITYHCKNSVAYMDQQTGNLKKALLLQGSNE | |
| | IEIRAEGNSRFTYSVTVDGCTSHTGAWGKTVIEYKTTKTSRLPIIDVAPLDVGAPDQEFG | |
| | FDVGPVCFL | |
| 5 | QCVNLTTRIQLPPAYTNSFTRGVYYPDKVFRSSVLHSTQDLFLPFFSNVIWFHAIHVSGT | Prototypic |
| | NGTKRFDNPVLPFNDGVYFASTEKSNIIRGWIFGTTLDSKTQSLLIVNNATNVVIKVCEF | SARS-CoV-2 |
| | QFCNDPFLGVYYHKNNKSWMESEFRVYSSANNCTFEYVSQPFLMDLEGKQGNFKNLREFV | spike |
| | FKNIDGYFKIYSKHTPINLVRDLPQGFSALEPLVDLPIGINITRFQTLLALHRSYLTPGD | NTD/RBD- |
| | SSSGWTAGAAAYYVGYLQPRTFLLKYNENGTITDAVDCALDPLSETKCTLKSFTVEKGIY | Trimer |
| | QTSNFRVQPTESIVRFPNITNLCPFGEVFNATRFASVYAWNRKRISNCVADYSVLYNSAS | fusion |
| | FSTFKCYGVSPTKLNDLCFTNVYADSFVIRGDEVRQIAPGQTGKIADYNYKLPDDFTGCV | polypeptide |
| | IAWNSNNLDSKVGGNYNYLYRLFRKSNLKPFERDISTEIYQAGSTPCNGVEGFNCYFPLQ | Witnout |
| | SYGFQPTNGVGYQPYRVVVLSFELLHAPATVCGPKKSTNLVKNKCRSNGLPGPIGPPGPR | signal |
| | GRTGDAGPVGPPGPPGPPGPPGPPSAGFDFSFLPQPPQEKAHDGGRYYRANDANVVRDRD | peptide, |
| | LEVDTTLKSLSQQIENIRSPEGSRKNPARTCRDLKMCHSDWKSGEYWIDPNQGCNLDAIK | 836 aa |
| | VECNMETGETCVYPTQPSVAOKNWYISKNPKDKRHVWFGESMTDGFQFEYGGQGSDPADV | |
| | AIQLTFLRLMSTEASQNITYHCKNSVAYMDQQTGNLKKALLLQGSNEIEIRAEGNSRFTY | |
| | SVTVDGCTSHTGAWGKTVIEYKTTKTSRLPIIDVAPLDVGAPDQEFGFDVGPVCFL | |
| 6 | QCVNLTTRTQLPPAYTNSFTRGVYYPDKVFRSSVLHSTQDLFLPFFSNVTWFHAIHVSGT | Prototypic |
| | NGTKRFDNPVLPFNDGVYFASTEKSNIIRGWIFGTTLDSKTQSLLIVNNATNVVIKVCEF | SARS-CoV-2 |
| | QFCNDPFLGVYYHKNNKSWMESEFRVYSSANNCTFEYVSQPFLMDLEGKQGNFKNLREFV | spike S1- |
| | FKNIDGYFKIYSKHTPINLVRDLPQGFSALEPLVDLPIGINITRFQTLLALHRSYLTPGD | Trimer |
| | SSSGWTAGAAAYYVGYLQPRTFLLKYNENGTITDAVDCALDPLSETKCTLKSFTVEKGIY | rus1on |
| | QTSNFRVQPTESIVRFPNITNLCPFGEVFNATRFASVYAWNRKRISNCVADYSVLYNSAS | polypeptide |
| | FSTFKCYGVSPTKLNDLCFTNVYADSFVIRGDEVRQIAPGQTGKIADYNYKLPDDFTGCV | without |
| | IAWNSNNLDSKVGGNYNYLYRLFRKSNLKPFERDISTEIYQAGSTPCNGVEGFNCYFPLQ | signal |
| | SYGFQPTNGVGYQPYRVVVLSFELLHAPATVCGPKKSTNLVKNKCVNFNFNGLTGTGVLT | peptide, |
| | ESNKKFLPFQQFGRDIADTTDAVRDPQTLEILDITPCSFGGVSVITPGTNTSNQVAVLYQ | 979 aa |
| | DVNCTEVPVAIHADQLTPTWRVYSTGSNVFQTRAGCLIGAEHVNNSYECDIPIGAGICAS | |
| | YQTQTNSPRSNGLPGPIGPPGPRGRTGDAGPVGPPGPPGPPGPPGPPSAGFDFSFLPQPP | |
| | QEKAHDGGRYYRANDANVVRDRDLEVDTTLKSLSQQIENIRSPEGSRKNPARTCRDLKMC | |
| | HSDWKSGEYWIDPNQGCNLDAIKVFCNMETGETCVYPTQPSVAQKNWYISKNPKDKRHVW | |
| | FGESMTDGFQFEYGGQGSDPADVALQLTFLRLMSTEASQNITYHCKNSVAYMDQQTGNLK | |
| | KALLLOGSNEIEIRAEGNSRFTYSVTVDGCTSHTGAWGKTVIEYKTTKTSRLPIIDVAPL | |
| | DVGAPDQEFGFDVGPVCFL | |
| 7 | SVASQSIIAYTMSLGAENSVAYSNNSIAIPTNFTISVTTEILPVSMTKTSVDCTMYICGD | Prototypic |
| | STECSNLLLQYGSFCTQLNRALTGIAVEQDKNTQEVFAQVKQIYKTPPIKDFGGFNFSQI | SARS-CoV-2 |
| | LPDPSKPSKRSFIEDLLFNKVTLADAGFIKQYGDCLGDIAARDLICAQKFNGLTVLPPLL | spike S2- |
| | TDEMIAQYTSALLAGTITSGWTFGAGAALQIPFAMQMAYRFNGIGVTQNVLYENQKLIAN | Trimer |
| | QFNSAIGKIQDSLSSTASALGKLQDVVNQNAQALNTLVKQLSSNFGAISSVLNDILSRLD | fusion |
| | KVEAEVQIDRLITGRLQSLQTYVTQQLIRAAEIRASANLAATKMSECVLGQSKRVDFCGK | polypeptide, |
| | GYHLMSFPQSAPHGVVFLHVTYVPAQEKNFTTAPAICHDGKAHFPREGVFVSNGTHWFVI | 837 aa |
| | QRNFYEPQIITTDNTFVSGNCDVVIGIVNNTVYDPLQPELDSFKEELDKYFKNHTSPDVD | (cleaved at |
| | LGDISGINASVVNIQKEIDRLNEVAKNLNESLIDLQELGKYEQYIKRSNGLPGPIGPPGP | S1/S2, site |
| | RGRTGDAGPVGPPGPPGPPGPPGPPSAGFDFSFLPQPPQEKAHDGGRYYRANDANVVRDR | 1) |
| | DLEVDTTLKSLSQQIENIRSPEGSRKNPARTCRDLKMCHSDWKSGEYWIDPNQGCNLDAI | |
| | KVFCNMETGETCVYPTQPSVAQKNWYISKNPKDKRHVWFGESMTDGFQFEYGGQGSDPAD | |
| | VAIQLTFLRLMSTEASQNITYHCKNSVAYMDQQTGNLKKALLLQGSNEIEIRAEGNSRFT | |
| | YSVTVDGCTSHTGAWGKTVIEYKTTKTSRLPIIDVAPLDVGAPDQEFGFDVGPVCFL | |
| 8 | TMSLGAENSVAYSNNSIAIPTNFTISVTTEILPVSMTKTSVDCTMYICGDSTECSNLLLQ | Prototypic |
| | YGSFCTQLNRALTGIAVEQDKNTQEVFAQVKQIYKTPPIKDFGGFNFSQILPDPSKPSKR | SARS-CoV-2 |
| | SFIEDLLFNKVTLADAGFIKQYGDCLGDIAARDLICAQKFNGLTVLPPLLTDEMIAQYTS | spike S2- |
| | ALLAGTITSGWTFGAGAALQIPFAMQMAYRFNGIGVTQNVLYENQKLIANQFNSAIGKIQ | Trimer |
| | DSLSSTASALGKLQDVVNQKAQALNTLVKQLSSNFGAISSVLNDILSRLDKVEAEVQIDR | fusion |
| | LITGRLQSLQTYVTQQLIRAAEIRASANLAATKMSECVLGQSKRVDFCGKGYHLMSFPQS | polypeptide, |
| | APHGVVFLHVTYVPAQEKNFTTAPAICHDGKAHFPREGVFVSNGTHWFVTORNFYEPQII | 827 aa |
| | TTDNTFVSGNCDVVIGIVNNTVYDPLQPELDSFKEELDKYFKNHTSPDVDLGDISGINAS | (cleaved at |
| | VVNIQKEIDRLNEVAKNLNESLIDLQELGKYEQYIKRSNGLPGPIGPPGPRGRTGDAGPV | S1/S2, site |
| | GPPGPPGPPGPPGPPSAGFDFSFLPQPPQEKAHDGGRYYRANDANVVRDRDLEVDTTLKS | 2) |
| | LSQQIENIRSPEGSRKNPARTCRDLKMCHSDWKSGEYWIDPNQGCNLDAIKVFCNMETGE | |
| | TCVYPTQPSVAQKNWYISKNPKDKRHVWFGESMTDGFQFEYGGQGSDPADVAIQLTFLRL | |
| | MSTEASQNITYHCKNSVAYMDQQTGNLKKALLLQGSNEIEIRAEGNSRFTYSVIVDGCTS | |
| | HTGAWGKTVIEYKTTKTSRLPIIDVAPLDVGAPDQEFGFDVGPVCFL | |
| 9 | SFIEDLLFNKVTLADAGFIKQYGDCLGDIAARDLICAQKFNGLTVLPPLLTDEMIAQYTS | Prototypic |
| | ALLAGTITSGWTFGAGAALQIPFAMQMAYRFNGIGVTQNVLYENQKLIANQFNSAIGKIQ | SARS-CoV-2 |
| | DSLSSTASALGKLQDVVNQNAQALNTLVKQLSSNFGAISSVLNDILSRLDKVEAEVQIDR | spike S2- |
| | LITGRLQSLQTYVTQQLIRAAEIBASANLAATKMSECVLGQSKRVDFCGKGYHLMSFPQS | Trimer |
| | APHGVVFLHVTYVPAQEKNFTTAPAICHDGKAHFPREGVFVSNGTHWFVTQRNFYEPQII | fusion |
| | TTDNTFVSGNCDVVIGIVNNTVYDPLQPELDSFKEELDKYFKNHTSPDVDLGDISGINAS | polypeptide, |
| | VVNIQKEIDRLNEVAKNLNESLIDLQELGKYEQYIKRSNGLPGPIGPPGPRGRTGDAGPV | 707 aa |
| | GPPGPPGPPGPPGPPSAGFDFSFLPQPPQEKARDGGRYYRANDANVVRDRDLEVDTTLKS | (cleaved at |
| | LSQQIENIRSPEGSRKNPARTCRDLKMCHSDWKSGEYWIDPNQGCNLDAIKVFCNMEIGE | S2′) |
| | TCVYPTQPSVAQKNWYISKNPKDKRHVWFGESMTDGFQFEYGGQGSDPADVAIQLTFLRL | |
| | MSTEASQNITYHCKNSVAYMDQQTGNLKKALLLQGSNEIEIRAEGNSRFTYSVTVDGCTS | |
| | HTGAWGKTVIEYKTTKTSRLPIIDVAPLDVGAPDQEFGFDVGPVCFL | |
| 10 | QCVNLTTRTQLPPAYTNSFTRGVYYPDKVFRSSVLHSTQDLFLPFFSNVTWFHAIHVSGT | B.1.351 |
| | NGTKRFDNPVLPFNDGVYFASTEKSNIIRGWIFGTTLDSKTQSLLIVNNATNVVIKVCEF | South |
| | QFCNDPFLGVYYHKNNKSWMESEFRVYSSANNCTFEYVSQPFLMDLEGKQGNFKNLREFV | African |
| | FKNIDGYFKIYSKHTPINLVRDLPQGFSALEPLVDLPIGINITRFQTLLALHRSYLTPGD | variant |
| | SSSGWTAGAAAYYVGYLQPRTFLLKYNENGTITDAVDCALDPLSETKCTLKSFTVEKGIY | SARS-CoV-2 |
| | QTSNFRVQPTESIVRFPNITNLCPFGEVFNATRFASVYAWNRKRISNCVADYSVLYNSAS | spike S- |
| | FSTFKCYGVSPTKLNDLCFTNVYADSFVIRGDEVRQIAPGQTGNIADYNYKLPDDFTGCV | Trimer |
| | IAWNSNNLDSKVGGNYNYLYRLFRKSNLKPFERDISTEIYQAGSTPCNGVKGFNCYFPLQ | polypept ide |
| | SYGFQPTYGVGYQPYRVVVLSFELLHAPATVCGPKKSTNLVKNKCVNFNFNGLTGTGVLT | without |
| | ESNKKFLPFQQFGRDIADTTDAVRDPQTLEILDITPCSFGGVSVITPGTNTSNQVAVLYQ | signal |
| | GVNCTEVPVAIHADQLTPTWRVYSTGSNVFQTRAGCLIGAEHVNNSYECDIPIGAGICAS | peptide, |
| | YQTQTNSPRRARSVASQSIIAYTMSLGAENSVAYSNNSIAIPTNFTISVTTEILPVSMTK | 1509 aa |
| | TSVDCTMYICGDSTECSNLLLQYGSFCTQLNRALTGIAVEQDKNTQEVFAQVKQIYKTPP | |
| | IKDFGGFNFSQILPDPSKPSKRSFIEDLLFNKVTLADAGFIKQYGDCLGDIAARDLICAQ | |
| | KFNGLTVLPPLLTDEMIAQYTSALLAGTITSGWTFGAGAALQIPFAMQMAYRFNGIGVTQ | |
| | NVLYENQKLIANQFNSAIGKIQDSLSSTASALGKLQDVVNQNAQALNTLVKQLSSNFGAI | |
| | SSVLNDILSRLDKVEAEVQIDRLITGRLQSLQTYVTQQLIBAAEIRASANLAATKMSECV | |
| | LGQSKRVDFCGKGYHLMSFPQSAPHGVVFLHVTYVPAQEKNFTTAPAICHDGKAHFPREG | |
| | VFVSNGTHWFVTQRNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQYIKRS | |
| | NGLPGPIGPPGPRGRTGDAGPVGPPGPPGPPGPPGPPSAGFDFSFLPQPPQEKAHDGGRY | |
| | YRANDANVVRDRDLEVDTTLKSLSQQIENIRSPEGSRKNPARTCRDLKMCHSDWKSGEYW | |
| | IDPNQGCNLDAIKVFCNMETGETCVYPTQPSVAQKNWYISKNPKDKRHVWFGESMTDGFQ | |
| | FEYGGQGSDPADVAIQLTFLRLMSTEASQNITYHCKNSVAYMDQQTGNLKKALLLQGSNE | |
| | IEIRAEGNSRFTYSVTVDGCTSHTGAWGKTVIEYKTTKTSRLPIIDVAPLDVGAPDQEFG | |
| | FDVGPVCFL | |
| 11 | QCVNLTTRTQLPPAYTNSFTRGVYYPDKVFRSSVLHSTQDLFLPFFSNVTWFHAIHVSGI | B.1.351 |
| | NGTKRFDNPVLPFNDGVYFASTEKSNIIRGWIFGTTLDSKTQSLLIVNNATNVVIKVCEF | South |
| | QFCNDPFLGVYYHKNNKSWMESEFRVYSSANNCTFEYVSQPFLMDLEGKQGNFKNLREFV | African |
| | FKNIDGYFKIYSKHTPINLVRDLPQGFSALEPLVDLPIGINITRFQTLLALHRSYLTPGD | variant |
| | SSSGWTAGAAAYYVGYLQPRTFLLKYNENGTITDAVDCALDPLSETKCTLKSFTVEKGIY | SARS-CoV-2 |
| | QTSNFRVQPTESIVRFPNITNLCPFGEVFNATRFASVYAWNRKRISNCVADYSVLYNSAS | spike S- |
| | FSTFKCYGVSPTKLNDLCFTNVYADSFVIRGDEVRQIAPGQTGNIADYNYKLPDDFTGCV | Trimer |
| | IAWNSNNLDSKVGGNYNYLYRLFRKSNLKPFERDISTEIYQAGSTPCNGVKGFNCYFPLQ | fusion |
| | SYGFQPTYGVGYQPYRVVVLSFELLHAPATVCGPKKSTNLVKNKCVNFNFNGLTGTGVLT | polypeptide |
| | ESNKKFLPFQQFGRDIADTTDAVRDPQTLEILDITPCSFGGVSVITPGTNTSNQVAVLYQ | without |
| | GVNCTEVPVAIHADQLTPTWRVYSTGSNVFQTRAGCLIGAEHVNNSYECDIPIGAGICAS | signal |
| | YQTQTNSPRRAASVASQSIIAYTMSLGAENSVAYSNNSIAIPTNFTISVTTEILPVSMTK | peptide, |
| | TSVDCTMYICGDSTECSNLLLQYGSFCTQLNRALTGIAVEQDKNTQEVFAQVKQIYKTPP | 1509 aa, |
| | IKDFGGFNFSQILPDPSKPSKRSFIEDLLFNKVTLADAGFIKQYGDCLGDIAARDLICAQ | S1/S2 furin |
| | KFNGLTVLPPLLTDEMIAQYTSALLAGTITSGWTFGAGAALQIPFAMQMAYRFNGIGVTQ | cleavage |
| | NVLYENQKL1ANQFNSAIGK1QDSLSSTASALGKLQDVVNQNAQALNTLVKQLSSNFGAI | site 1 |
| | SSVLNDILSRLDKVEAEVQIDRLITGRLQSLQTYVTQQLIRAAEIRASANLAATKMSECV | mutant |
| | LGQSKRVDFCGKGYHLMSFPQSAPHGVVFLHVTYVPAQEKNFTTAPAICHDGKAHFPREG | (685R→685A) |
| | VFVSNGTHWFVTQRNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQYIKRS | |
| | NGLPGPIGPPGPRGRTGDAGPVGPPGPPGPPGPPGPPSAGFDFSFLPQPPQEKAHDGGRY | |
| | YRANDANVVRDRDLEVDTTLKSLSQQIENIRSPEGSRKNPARTCRDLKMCHSDWKSGEYW | |
| | IDPNQGCNLDAIKVFCNMETGETCVYPTOPSVAQKNWYISKNPKDKRHVWFGESMTDGFQ | |
| | FEYGGQGSDPADVAIQLTFLRLMSTEASQNITYHCKNSVAYMDQQTGNLKKALLLQGSNE | |
| | IEIRAEGNSRFTYSVTVDGCTSHTGAWGKTVIEYKTTKTSRLPIIDVAPLDVGAPDQEFG | |
| | FDVGPVCFL | |
| 12 | QCVNLTTRTQLPPAYTNSFTRGVYYPDKVFRSSVLHSTQDLFLPFFSNVTWFHAIHVSGT | B.1.351 |
| | NGTKRFDNPVLPFNDGVYFASTEKSNIIRGWIFGTTLDSKTQSLLIVNNATNVVIKVCEF | South |
| | QFCNDPFLGVYYHKNNKSWMESEFRVYSSANNCTFEYVSQPFLMDLEGKQGNFKNLREFV | African |
| | FKNIDGYFKIYSKHTPINLVRDLPQGFSALEPLVDLPIGINITRFQTLLALHRSYLTPGD | variant |
| | SSSGWTAGAAAYYVGYLQPRTFLLKYNENGTITDAVDCALDPLSETKCTLKSFTVEKGIY | SARS-CoV-2 |
| | QTSNFRVQPTESIVRFPNITNLCPFGEVFNATRFASVYAWNRKRISNCVADYSVLYNSAS | spike S- |
| | FSTFKCYGVSPTKLNDLCFTNVYADSFVIRGDEVRQIAPGQTGNIADYNYKLPDDFTGCV | Trimer |
| | IAWNSNNLDSKVGGNYNYLYRLFRKSNLKPFERDISTEIYQAGSTPCNGVKGFNCYFPLQ | fusion |
| | SYGFQPTYGVGYOPYRVVVLSFELLHAPATVCGPKKSTNLVKNKCVNFNFNGLIGTGVLT | polypeptide |
| | ESNKKFLPFQQFGRDIADTTDAVRDPQTLEILD1TPCSFGGVSVITPGTNTSNQVAVLYQ | without |
| | GVNCTEVPVAIHADQLTPTWRVYSTGSNVFQTRAGCLIGAEHVNNSYECDIPIGAGICAS | signal |
| | YQTQTNSPRRARSVASQSIIAYTMSLGAENSVAYSNNS1AIPTNFTISVTTEILPVSMTK | peptide, |
| | TSVDCTMYICGDSTECSNLLLQYGSFCTQLNRALTGIAVEQDKNTQEVFAQVKQIYKTPP | 1509 aa, |
| | IKDFGGFNFSQILPDPSKPSKRSFIEDLLFNKVTLADAGFIKQYGDCLGDIAARDLICAQ | proline |
| | KFNGLTVLPPLLIDEMIAQYTSALLAGTITSGWTFGAGAALQIPFAMQMAYRFNGIGVTQ | mutant |
| | NVLYENQKLIANQFNSAIGKIQDSLSSTASALGKLQDVVNQNAQALNTLVKQLSSNFGAI | (986K/987V→ |
| | SSVLNDILSRLDPPEAEVQIDRLITGRLQSLQTYVTQQLIRAAEIRASANLAATKMSECV | 986P/987P) |
| | LGQSKRVDFCGKGYHLMSFPQSAPHGVVFLHVTYVPAQEKNFTTAPAICHDGKAHFPREG | |
| | VFVSNGTHWFVTQRNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQYIKRS | |
| | NGLPGPIGPPGPRGRTGDAGPVGPPGPPGPPGPPGPPSAGFDFSFLPQPPQEKAHDGGRY | |
| | YRANDANVVRDRDLEVDTTLKSLSQQIENIRSPEGSRKNPARTCRDLKMCHSDWKSGEYW | |
| | IDPNQGCNLDAIKVFCNMETGETCVYPTQPSVAQKNWYISKNPKDKRHVWFGESMTDGFQ | |
| | FEYGGQGSDPADVAIQLTFLRLMSTEASQNITYHCKNSVAYMDQQTGNLKKALLLQGSNE | |
| | IEIRAEGNSRFTYSVTVDGCTSHTGAWGKTVIEYKTTKTSRLPIIDVAPLDVGAPDQEFG | |
| | FDVGPVCFL | |
| 13 | QCVNLTTRTQLPPAYTNSFTRGVYYPDKVFRSSVLHSTQDLFLPFFSNVTWFHAIHVSGT | B.1.351 |
| | NGTKRFDNPVLPFNDGVYFASTEKSNIIRGWIFGTTLDSKTQSLLIVNNATNVVIKVCEE | South |
| | QFCNDPFLGVYYHKNNKSWMESEFRVYSSANNCTFEYVSQPFLMDLEGKQGNFKNLREFV | African |
| | FKNIDGYFKIYSKHTPINLVRDLPQGFSALEPLVDLPIGINITRFQTLLALHRSYLTPGD | variant |
| | SSSGWTAGAAAYYVGYLQPRTFLLKYNENGTITDAVDCALDPLSETKCTLKSFTVEKGIY | SARS-CoV-2 |
| | QTSNFRVQPTESIVRFPNITNLCPFGEVFNATRFASVYAWNRKRISNCVADYSVLYNSAS | spike S- |
| | FSTFKCYGVSPTKLNDLCFTNVYADSFVIRGDEVRQIAPGQTGNIADYNYKLPDDFTGCV | Trimer |
| | IAWNSNNLDSKVGGNYNYLYRLFRKSNLKPFERDISTEIYQAGSTPCNGVKGFNCYFPLQ | fusion |
| | SYGFQPTYGVGYQPYRVVVLSFELLHAPATVCGPKKSTNLVKNKCVNFNFNGLTGIGVLI | polypeptide |
| | ESNKKFLPFQQFGRDIADTTDAVRDPQTLEILDITPCSFGGVSVITPGTNTSNQVAVLYQ | without |
| | GVNCTEVPVAIHADQLTPTWRVYSTGSNVFQTRAGCLIGAEHVNNSYECDIPIGAGICAS | signal |
| | YQTQINSPRRAASVASQSIIAYTMSLGAENSVAYSNNSIAIPTNFTISVTTEILPVSMTK | peptide, |
| | TSVDCTMYICGDSTECSNLLLQYGSFCTQLNRALTGIAVEQDKNTQEVFAQVKQIYKTPP | 1509 aa, |
| | IKDFGGFNFSQILPDPSKPSKRSFIEDLLFNKVTLADAGFIKQYGDCLGDIAARDLICAQ | S1/S2 furin |
| | KFNGLTVLPPLLTDEMIAQYTSALLAGTITSGWTFGAGAALQIPFAMQMAYRFNGIGVTQ | cleavage |
| | NVLYENQKLIANQFNSAIGKIQDSLSSTASALGKLQDVVNQNAQALNTLVKQLSSNFGAI | site 1 and |
| | SSVLNDILSRLDPPEAEVQIDRLITGRLQSLQTYVTQQLIRAAEIRASANLAATKMSECV | proline |
| | LGQSKRVDFCGKGYHLMSFPQSAPHGVVFLHVTYVPAQEKNFTTAPAICHDGKAHFPREG | mutant |
| | VFVSNGTHWFVTQRNFYEPQIITTDNTFVSGNCDVVIGIVNNTVYDPLQPELDSFKEELD | (685R→685A, |
| | KYFKNHTSPDVDLGDISGINASVVNIQKEIDRLNEVAKNLNESLIDLQELGKYEQYIKRS | 986K/987V→ |
| | NGLPGPIGPPGPRGRTGDAGPVGPPGPPGPPGPPGPPSAGFDFSFLPQPPQEKAHDGGRY | 986P/987P) |
| | YRANDANVVRDRDLEVDTTLKSLSQQIENIRSPEGSRKNPARTCRDLKMCHSDWKSGEYW | |
| | IDPNQGCNLDAIKVFCNMETGETCVYPTQPSVAQKNWYISKNPKDKRRVWFGESMTDGFQ | |
| | FEYGGQGSDPADVAIQLTFLRLMSTEASQNITYHCKNSVAYMDQQTGNLKKALLLQGSNE | |
| | IEIRAEGNSRFTYSVTVDGCTSHTGAWGKTVIEYKTTKTSRLPIIDVAPLDVGAPDQEFG | |
| | FDVGPVCFL | |
| 14 | QCVNFTNRTQLPSAYTNSFTRGVYYPDKVFRSSVLHSTQDLFLPFFSNVTWFHAIHVSGT | P.1 |
| | NGTKRFDNPVLPFNDGVYFASTEKSNIIRGWIFGTTLDSKTQSLLIVNNATNVVIKVCEF | Brazilian |
| | QFCNYPFLGVYYHKNNKSWMESEFRVYSSANNCTFEYVSQPFLMDLEGKQGNFKNLSEFV | variant |
| | FKNIDGYFKIYSKHTPINLVRDLPQGFSALEPLVDLPIGINITRFQTLLALHRSYLTPGD | SARS-CoV-2 |
| | SSSGWTAGAAAYYVGYLQPRTFLLKYNENGTITDAVDCALDPLSETKCTLKSFTVEKGIY | spike S- |
| | QTSNFRVQPTESIVRFPNITNLCPFGEVFNATRFASVYAWNRKRISNCVADYSVLYNSAS | Trimer |
| | FSTFKCYGVSPTKLNDLCFTNVYADSFVIRGDEVRQIAPGQTGTIADYNYKLPDDFTGCV | fusion |
| | IAWNSNNLDSKVGGNYNYLYRLFRKSNLKPFERDISTEIYQAGSTPCNGVKGFNCYFPLQ | polypeptide |
| | SYGFQPTYGVGYOPYRVVVLSFELLHAPATVCGPKKSTNLVKNKCVNFNFNGLIGTGVLT | without |
| | ESNKKFLPFQQFGRDIADTTDAVRDPQTLEILDITPCSFGGVSVITPGTNTSNQVAVLYQ | signal |
| | GVNCTEVPVAIHADQLTPTWRVYSTGSNVFQTRAGCLIGAEYVNNSYECDIPIGAGICAS | peptide, |
| | YQTQTNSPRRARSVASQSIIAYTMSLGAENSVAYSNNSIAIPTNFTISVTTEILPVSMTK | 1509 aa |
| | TSVDCTMYICGDSTECSNLLLQYGSFCTQLNRALTGIAVEQDKNTQEVFAQVKQIYKTPP | |
| | IKDFGGFNFSQILPDPSKPSKRSFIEDLLFNKVTLADAGFIKQYGDCLGDIAARDLICAQ | |
| | KFNGLTVLPPLLTDEMIAQYTSALLAGTITSGWTFGAGAALQIPFAMQMAYRFNGIGVTQ | |
| | NVLYENQKLIANQFNSAIGKIQDSLSSTASALGKLQDVVNQNAQALNTLVKQLSSNFGAI | |
| | SSVLNDILSRLDKVEAEVQIDRLITGRLQSLQTYVTQQLIRAAEIRASANLAAIKMSECV | |
| | LGQSKRVDFCGKGYHLMSFPQSAPHGVVFLHVTYVPAQEKNFTTAPAICHDGKAHFPREG | |
| | VFVSNGTHWFVTQRNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQYIKRS | |
| | NGLPGPIGPPGPRGRTGDAGPVGPPGPPGPPGPPGPPSAGFDFSFLPQPPQEKAHDGGRY | |
| | YRANDANVVRDRDLEVDTTLKSLSQQIENIRSPEGSRKNPARTCRDLKMCHSDWKSGEYW | |
| | IDPNQGCNLDAIKVFCNMETGETCVYPTQPSVAQKNWYISKNPKDKRHVWFGESMTDGFQ | |
| | FEYGGQGSDPADVAIQLTFLRLMSTEASQNITYHCKNSVAYMDQQTGNLKKALLLQGSNE | |
| | IEIRAEGNSRFTYSVTVDGCTSHTGAWGKTVIEYKTTKTSRLPIIDVAPLDVGAPDQEFG | |
| | FDVGPVCFL | |
| 15 | QCVNFTNRTQLPSAYTNSFTRGVYYPDKVFRSSVLHSTQDLFLPFFSNVTWFHAIHVSGT | P.1 |
| | NGTKRFDNPVLPFNDGVYFASTEKSNIIRGWIFGTTLDSKTQSLLIVNNATNVVIKVCEE | Brazilian |
| | QFCNYPFLGVYYHKNNKSWMESEFRVYSSANNCTFEYVSQPFLMDLEGKQGNFKNLSEFV | variant |
| | FKNIDGYFKIYSKHTPINLVRDLPQGFSALEPLVDLPIGINITRFQTLLALHRSYLTPGD | SARS-CoV-2 |
| | SSSGWTAGAAAYYVGYLQPRTFLLKYNENGTITDAVDCALDPLSETKCTLKSFTVEKGIY | spike S- |
| | QTSNFRVQPTESIVRFPNITNLCPFGEVFNATRFASVYAWNRKRISNCVADYSVLYNSAS | Trimer |
| | FSTFKCYGVSPTKLNDLCFTNVYADSFVIRGDEVRQIAPGQTGTIADYNYKLPDDFTGCV | fusion |
| | IAWNSNNLDSKVGGNYNYLYRLFRKSNLKPFERDISTEIYQAGSTPCNGVKGFNCYFPLQ | polypeptide |
| | SYGFQPTYGVGYQPYRVVVLSFELLHAPATVCGPKKSTNLVKNKCVNFNFNGLTGTGVLI | without |
| | ESNKKFLPFQQFGRDIADTTDAVRDPQTLEILDITPCSFGGVSVITPGTNTSNQVAVLYQ | signal |
| | GVNCTEVPVAIHADQLTPTWRVYSTGSNVFQTRAGCLIGAEYVNNSYECDIPIGAGICAS | peptide, |
| | YQTQTNSPRRAASVASQSIIAYTMSLGAENSVAYSNNSIAIPTNFTISVTTEILPVSMTK | 1509 aa, |
| | TSVDCTMYICGDSTECSNLLLQYGSFCTQLNRALTGIAVEQDKNTQEVFAQVKQIYKIPP | S1/S2 furin |
| | IKDFGGFNFSQILPDPSKPSKRSFIEDLLFNKVTLADAGFIKQYGDCLGDIAARDLICAQ | cleavage |
| | KFNGLTVLPPLLTDEMIAQYTSALLAGTITSGWTFGAGAALQIPFAMQMAYRFNGIGVTQ | site 1 |
| | NVLYENQKLIANQFNSAIGKIQDSLSSTASALGKLQDVVNQNAQALNTLVKQLSSNFGAI | mutant |
| | SSVLNDILSRLJKVEAEVQIDRLITGHLQSLQTYVTQQLIRAAEIBASANLAAIKWSECV | (685R→685A) |
| | LGQSKRVDFCGKGYHLMSFPQSAPHGVVFLHVTYVPAQEKNFTTAPAICHDGKAHFPREG | |
| | VFVSNGTHWFVTORNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQYIKRS | |
| | NGLPGPIGPPGPRGRTGDAGPVGPPGPPGPPGPPGPPSAGFDFSFLPQPPQEKAHDGGRY | |
| | YRANDANVVRDRDLEVDTTLKSLSQQIENIRSPEGSRKNPARTCRDLKMCHSDWKSGEYW | |
| | IDPNQGCNLDAIKVFCNMETGETCVYPTQPSVAQKNWYISKNPKDKRHVWFGESMTDGFQ | |
| | FEYGGQGSDPADVAIQLTFLRLMSTEASQNITYACKNSVAYMDQQTGNLKKALLLQGSNE | |
| | IEIRAEGNSRFTYSVTVDGCTSHIGAWGKTVIEYKTTKTSRLPIIDVAPLDVGAPDQEFG | |
| | FDVGPVCFL | |
| 16 | QCVNFTNRTQLPSAYTNSFTRGVYYPDKVFRSSVLHSTQDLFLPFFSNVTWFHAIHVSGT | P.1 |
| | NGTKRFDNPVLPFNDGVYFASTEKSNIIRGWIFGTTLDSKTQSLLIVNNATNVVIKVCEF | Brazilian |
| | QFCNYPFLGVYYHKNNKSWMESEFRVYSSANNCTFEYVSQPFLMDLEGKQGNFKNLSEFV | variant |
| | FKNIDGYFKIYSKHTPINLVRDLPQGFSALEPLVDLPIGINITRFQTLLALHRSYLTPGD | SARS-CoV-2 |
| | SSSGWTAGAAAYYVGYLQPRTFLLKYNENGTITDAVDCALDPLSETKCTLKSFTVEKGIY | spike S- |
| | QTSNFRVOPTESIVRFPNITNLCPFGEVFNATRFASVYAWNRKRISNCVADYSVLYNSAS | Trimer |
| | FSTFKCYGVSPTKLNDLCFTNVYADSFVIRGDEVRQIAPGQTGTIADYNYKLPDDFTGCV | fusion |
| | IAWNSNNLDSKVGGNYNYLYRLFRKSNLKPFERDISTEIYQAGSTPCNGVKGFNCYFPLQ | polypeptide |
| | SYGFQPTYGVGYOPYRVVVLSFELLHAPATVCGPKKSTNLVKNKCVNFNFNGLIGTGVLT | without |
| | ESNKKFLPFQQFGRDIADTTDAVRDPQTLEILDITPCSFGGVSVITPGTNTSNQVAVLYQ | signal |
| | GVNCTEVPVAIHADQLTPTWRVYSTGSNVFQTRAGCLIGAEYVNNSYECDIPIGAGICAS | peptide, |
| | YQTQTNSPRRARSVASQSIIAYTMSLGAENSVAYSNNS1AIPTNFTISVTTEILPVSMTK | 1509 aa, |
| | TSVDCTMYICGDSTECSNLLLQYGSFCTQLNRALTGIAVEQDKNTQEVFAQVKQIYKTPP | proline |
| | IKDFGGFNFSQILPDPSKPSKRSFIEDLLFNKVTLADAGIIKQYGDCLGDIAARDLICAQ | mutant |
| | KFNGLTVLPPLLTDEMIAQYTSALLAGTITSGWTFGAGAALQIPFAMQMAYRFNGIGVTQ | (986K/987V→ |
| | NVLYENQKLIANQFNSAIGKIQDSLSSTASALGKLQDVVNQNAQALNTLVKQLSSNFGAI | 986P/987P) |
| | SSVLNDILSRLDPPEAEVQIDRLITGRLQSLQTYVTQQLIRAAEIRASANLAAIKMSECV | |
| | LGQSKRVDFCGKGYHLMSFPQSAPHGVVFLHVTYVPAQEKNFTTAPAICHDGKAHFPREG | |
| | VFVSNGTHWFVTQRNFYEPQIITTDNTFVSGNCDVVIGIVNNTVYDPLQPELDSFKEELD | |
| | KYFKNHISPDVDLGDISGINASVVNIQKEIDRLNEVAKNLNESLIDLQELGKYEQYIKRS | |
| | NGLPGPIGPPGPRGRTGDAGPVGPPGPPGPPGPPGPPSAGFDFSFLPQPPQEKAHDGGRY | |
| | YRANDANVVRDRDLEVDTTLKSLSQQIENIRSPEGSRKNPARTCRDLKMCHSDWKSGEYW | |
| | IDPNQGCNLDAIKVFCNMETGETCVYPTQPSVAQKNWYISKNPKDKRHVWFGESMTDGFQ | |
| | FEYGGQGSDPADVAIQLTFLRLMSTEASQNITYHCKNSVAYMDQQTGNLKKALLLQGSNE | |
| | IEIRAEGNSRFTYSVTVDGCTSHTGAWGKTVIEYKTTKTSRLPIIDVAPLDVGAPDQEFG | |
| | FDVGPVCFL | |
| 17 | QCVNFTNRTQLPSAYTNSFTRGVYYPDKVFRSSVLHSTQDLFLPFFSNVTWFHAIHVSGT | P.1 |
| | NGTKRFDNPVLPFNDGVYFASTEKSNIIRGWIFGTTLDSKTQSLLIVNNATNVVIKVCEF | Brazilian |
| | QFCNYPFLGVYYHKNNKSWMESEFRVYSSANNCTFEYVSQPFLMDLEGKQGNFKNLSEFV | variant |
| | FKNIDGYFKIYSKHTPINLVRDLPQGFSALEPLVDLPIGINITRFQTLLALHRSYLTPGD | SARS-CoV-2 |
| | SSSGWTAGAAAYYVGYLQPRTFLLKYNENGTITDAVDCALDPLSETKCTLKSFTVEKGIY | spike S- |
| | QTSNFRVQPTESIVRFPNITNLCPFGEVFNATRFASVYAWNRKRISNCVADYSVLYNSAS | Trimer |
| | FSTFKCYGVSPTKLNDLCFTNVYADSFVIRGDEVRQIAPGQTGTIADYNYKLPDDFTGCV | polypeptide |
| | IAWNSNNLDSKVGGNYNYLYRLFRKSNLKPFERDISTEIYQAGSTPCNGVKGFNCYFPLQ | without |
| | SYGFQPTYGVGYQPYRVVVLSFELLHAPATVCGPKKSTNLVKNKCVNFNFNGLTGTGVLT | signal |
| | ESNKKFLPFQQFGRDIADTTDAVRDPQTLEILDITPCSFGGVSVITPGTNTSNQVAVLYQ | peptide, |
| | GVNCTEVPVAIHADQLTPTWRVYSTGSNVFQTRAGCLIGAEYVNNSYECDIPIGAGICAS | 1509 aa, |
| | YQTQTNSPRRAASVASQSIIAYTMSLGAENSVAYSNNSIAIPTNFTISVTTEILPVSMTK | S1/S2 furin |
| | TSVDCTMYICGDSTECSNLLLQYGSFCTQLNRALTGIAVEQDKNTQEVFAQVKQIYKIPP | cleavage |
| | IKDFGGFNFSQILPDPSKPSKRSFIEDLLFNKVTLADAGFIKQYGDCLGDIAARDLICAQ | site 1 and |
| | KFNGLTVLPPLLTDEMIAQYTSALLAGTITSGWIHGAGAALQIPFAMQMAYRFNGIGVTQ | proline |
| | NVLYENQKLIANQFNSAIGKIQDSLSSTASALGKLQDVVNQNAQALNTLVKQLSSNFGAI | mutant |
| | SSVLNDILSRLDPPEAEVQIDRLITGRLQSLQTYVTQQLIBAAEIRASANLAAIKMSECV | (685R→685A, |
| | LGQSKRVDFCGKGYHLMSFPQSAPHGVVFLHVTYVPAQEKNFTTAPAICHDGKAHFPREG | 986K/987V→ |
| | VFVSNGTHWFVTQRNFYEPQIITTDNTFVSGNCDVVIGIVNNTVYDPLQPELDSFKEELD | 986P/987P) |
| | KYFKNHTSPDVDLGDISGINASVVNIQKEIDRLNEVAKNLNESLIDLQELGKYEQYIKRS | |
| | NGLPGPIGPPGPRGRTGDAGPVGPPGPPGPPGPPGPPSAGFDFSFLPQPPQEKAHDGGRY | |
| | YRANDANVVRDRDLEVDTTLKSLSQQIENIRSPEGSRKNPARTCRDLKMCHSDWKSGEYW | |
| | IDPNQGCNLDAIKVFCNMETGETCVYPTQPSVAQKNWYISKNPKDKRHVWFGESMTDGFQ | |
| | FEYGGQGSDPADVAIQLTFLRLMSTEASQNITYHCKNSVAYMDQQTGNLKKALLLQGSNE | |
| | IEIRAEGNSRFTYSVTVDGCTSHIGAWGKTVIEYKTTKISRLPIIDVAPLDVGAPDQEFG | |
| | FDVGPVCFL | |
| 18 | QCVNLTTRTQLPPAYTNSFTRGVYYPDKVFRSSVLHSTQDLFLPFFSNVTWFHAISGTNG | B.1.1.7 UK |
| | TKRFDNPVLPFNDGVYFASTEKSNIIRGWIFGTTLDSKTQSLLIVNNATNVVIKVCEFQF | variant |
| | CNDPFLGVYYHKNNKSWMESEFRVYSSANNCTFEYVSQPFLMDLEGKQGNFKNLREFVFK | SARS-CoV-2 |
| | NIDGYFKIYSKHIPINLVRDLPQGFSALEPLVDLPIGINITRFQTLLALHRSYLTPGDSS | spike S- |
| | SGWTAGAAAYYVGYLQPRTFLLKYNENGTITDAVDCALDPLSETKCTLKSFTVEKGIYQT | Trimer |
| | SNFRVQPTESIVRFPNIINLCPFGEVFNATRFASVYAWNRKRISNCVADYSVLYNSASFS | fusion |
| | TFKCYGVSPTKLNDLCFTNVYADSFVIRGDEVRQIAPGQTGKIADYNYKLPDDFTGCVIA | polypeptide |
| | WNSNNLDSKVGGNYNYLYRLFRKSNLKPFERDISTEIYQAGSTPCNGVEGFNCYFPLQSY | without |
| | GFQPTYGVGYQPYRVVVLSFELLHAPATVCGPKKSTNLVKNKCVNFNFNGLTGTGVLTES | signal |
| | NKKFLPFQQFGRDIADTTDAVRDPQTLEILDITPCSFGGVSVITPGTNTSNQVAVLYQGV | peptide, |
| | NCTEVPVAIHADQLTPTWRVYSTGSNVFQTRAGCLIGAEHVNNSYECDIPIGAGICASYQ | 1507 aa |
| | TQTNSHRRARSVASQSIIAYTMSLGAENSVAYSNNSIAIPTNFTISVTTEILPVSMTKTS | |
| | VDCTMYICGDSTECSNLLLQYGSFCTQLNRALTGIAVEQDKNTQEVFAQVKQIYKTPPIK | |
| | DFGGFNFSQILPDPSKPSKRSFIEDLLFNKVTLADAGFIKQYGDCLGDIAARDLICAQKF | |
| | NGLTVLPPLLTDEMIAQYTSALLAGTITSGWTFGAGAALQIPFAMQMAYRFNGIGVTQNV | |
| | LYENQKLIANQFNSAIGKIQDSLSSTASALGKLQDVVNQNAQALNTLVKQLSSNFGAISS | |
| | VLNDILSRLDKVEAEVQIDRLITGRLQSLQTYVTQQLIRAAEIRASANLAATKMSECVLG | |
| | QSKRVDFCGKGYHLMSFPQSAPHGVVFLHVTYVPAQEKNFTTAPAICHDGKAHFPREGVF | |
| | VSNGTHWFVTQRNFYEPQIITTDNTFVSGNCDVVIGIVNNTVYDPLQPELDSFKEELDKY | |
| | FKNHTSPDVDLGDISGINASVVNIQKEIDRLNEVAKNLNESLIDLQELGKYEQYIKRSNG | |
| | LPGPIGPPGPRGRTGDAGPVGPPGPPGPPGPPGPPSAGFDFSFLPQPPQEKAHDGGRYYR | |
| | ANDANVVRDRDLEVDTTLKSLSQQIENIRSPEGSRKNPARTCRDLKMCHSDWKSGEYWID | |
| | PNQGCNLDAIKVFCNMETGETCVYPTQPSVAQKNWYISKNPKDKRHVWFGESMTDGFQFE | |
| | YGGQGSDPADVAIQLTFLRLMSTEASQNITYHCKNSVAYMDQQTGNLKKALLLQGSNEIE | |
| | IRAEGNSRFTYSVTVDGCTSHTGAWGKTVIEYKTTKTSRLPIIDVAPLDVGAPDQEFGFD | |
| | VGPVCFL | |
| 19 | QCVNLTTRTQLPPAYTNSFTRGVYYPDKVFRSSVLHSTQDLFLPFFSNVTWFHAISGTNG | B.1.1.7 UK |
| | TKRFDNPVLPFNDGVYFASTEKSNIIRGWIFGTTLDSKTQSLLIVNNATNVVIKVCEFQF | variant |
| | CNDPFLGVYYHKNNKSWMESEFRVYSSANNCTFEYVSQPFLMDLEGKQGNFKNLREFVFK | SARS-CoV-2 |
| | NIDGYFKIYSKHTPINLVRDLPQGFSALEPLVDLPIGINITRFQTLLALHRSYLTPGDSS | spike S- |
| | SGWTAGAAAYYVGYLQPRTFLLKYNENGTITDAVDCALDPLSETKCTLKSFTVEKGIYQT | Trimer |
| | SNFRVQPTESIVRFPNITNLCPFGEVFNATRFASVYAWNRKRISNCVADYSVLYNSASFS | fusion |
| | TFKCYGVSPTKLNDLCFTNVYADSFVIRGDEVRQIAPGQTGKIADYNYKLPDDFTGCVIA | polypeptide |
| | WNSNNLDSKVGGNYNYLYRLFRKSNLKPFERDISTEIYQAGSTPCNGVEGFNCYFPLQSY | without |
| | GFQPTYGVGYQPYRVVVLSFELLHAPATVCGPKKSTNLVKNKCVNFNFNGLTGIGVLTES | signal |
| | NKKFLPFQQFGRDIADTTDAVRDPQTLEILDITPCSFGGVSVITPGTNTSNQVAVLYQGV | peptide, |
| | NCTEVPVAIHADQLTPTWRVYSTGSNVFQTRAGCLIGAEHVNNSYECDIPIGAGICASYQ | 1507 aa, |
| | TQTNSHRRAASVASQSIIAYTMSLGAENSVAYSNNSIAIPTNFTISVTTEILPVSMTKTS | S1/S2 furin |
| | VDCTMYICGDSTECSNLLLQYGSFCTQLNRALTGIAVEQDKNTQEVFAQVKQIYKTPPIK | cleavage |
| | DFGGFNFSQILPDPSKPSKRSFIEDLLFNKVTLADAGL’RVYGDCLGDIAARDLICAQKF | site 1 |
| | NGLTVLPPLLTDEMIAQYTSALLAGTITSGWTFGAGAALQIPFAMQMAYRFNGIGVTQNV | mutant |
| | LYENQKLIANQFNSAIGKIQDSLSSTASALGKLQDVVNQNAQALNTLVKQLSSNFGAISS | (685R→685A) |
| | VLNDILSRLDKVEAEVQIDRLITGRLQSLMTYVIQQLIRAAEIRASANLAATKMSECVLG | |
| | QSKRVDFCGKGYHLMSFPQSAPHGVVFLHVTYVPAQEKNFTTAPAICHDGKAHFPREGVF | |
| | VSNGTHWFVTQRNFYEPQIITTDNTFVSGNCDVVIGIVNNTVYDPLQPELDSFKEELDKY | |
| | FKNHTSPDVDLGDISGINASVVNIQKEIDRLNEVAKNLNESLIDLQELGKYEQYIKRSNG | |
| | LPGPIGPPGPRGRTGDAGPVGPPGPPGPPGPPGPPSAGFDFSFLPQPPQEKAHDGGRYYR | |
| | ANDANVVRDRDLEVDTTLKSLSQQIENIRSPEGSRKNPARTCRDLKMCHSDWKSGEYWID | |
| | PNQGCNLDAIKVFCNMETGETCVYPTQPSVAQKNWYISKNPKDKRHVWFGESMTDGFQFE | |
| | YGGQGSDPADVAIQLTFLRLMSTEASQNITYHCKNSVAYMDQQTGNLKKALLLQGSNEIE | |
| | IRAEGNSRFTYSVTVDGCTSHTGAWGKTVIEYKTTKTSRLPIIDVAPLDVGAPDQEFGFD | |
| | VGPVCPL | |
| 20 | QCVNLTTRTQLPPAYTNSFTRGVYYPDKVFRSSVLHSTQDLFLPFFSNVTWFHAISGING | B.1.1.7 UK |
| | TKRFDNPVLPFNDGVYFASTEKSNIIRGWIFGTTLDSKTQSLLIVNNATNVVIKVCEFQF | variant |
| | CNDPFLGVYYHKNNKSWMESEFRVYSSANNCTFEYVSQPFLMDLEGKQGNFKNLREFVFK | SARS-CoV-2 |
| | NIDGYFKIYSKHTPINLVRDLPQGFSALEPLVDLPIGINITRFQILLALHRSYLTPGDSS | spike S- |
| | SGWTAGAAAYYVGYLQPRTFLLKYNENGTITDAVDCALDPLSETKCTLKSFTVEKGIYQT | Trimer |
| | SNFRVQPTESIVRFPNITNLCPFGEVFNATRFASVYAWNRKRISNCVADYSVLYNSASFS | fusion |
| | TFKCYGVSPTKLNDLCFTNVYADSFVIRGDEVRQIAPGQTGKIADYNYKLPDDFTGCVIA | polypeptide |
| | WNSNNLDSKVGGNYNYLYRLFRKSNLKPFERDISTEIYQAGSTPCNGVEGFNCYFPLQSY | witnout |
| | GFQPTYGVGYQPYRVVVLSFELLHAPATVCGPKKSTNLVKNKCVNFNFNGLTGTGVLTES | signal |
| | NKKFLPFQQFGRDIADTTDAVRDPQTLEILDITPCSFGGVSVITPGTNTSNQVAVLYOGV | peptide, |
| | NCTEVPVAIHADQLTPTWRVYSTGSNVFQTRAGCLIGAEHVNNSYECDIPIGAGICASYQ | 1507 aa, |
| | TQTNSHRRARSVASQSIIAYTMSLGAENSVAYSNNSIAIPTNFTISVTTEILPVSMTKTS | proline |
| | VDCTMYICGDSTECSNLLLQYGSFCTQLNRALTGIAVEQDKNTQEVFAQVKQIYKTPPIK | mutant |
| | DFGGFNFSQILPDPSKPSKRSFIEDLLFNKVTLADAGFIKQYGDCLGDIAARDLICAQKF | (986K/987V→ |
| | NGLTVLPPLLTDEMIAQYTSALLAGTITSGWTFGAGAALQIPFAMQMAYRFNGIGVTQNV | 986P/987P) |
| | LYENQKLIANQFNSAIGKIQDSLSSTASALGKLQDVVNQNAQALNTLVKQLSSNFGAISS | |
| | VLNDILSRLDPPEAEVQIDRLITGRLQSLQTYVTQQLIRAAEIRASANLAATKMSECVLG | |
| | QSKRVDFCGKGYHLMSFPQSAPHGVVFLHVTYVPAQEKNFTTAPAICHDGKAHFPREGVF | |
| | VSNGTHWFVTQRNFYEPQIITTDNTFVSGNCDVVIGIVNNTVYDPLQPELDSFKEELDKY | |
| | FKNHTSPDVDLGDISGINASVVNIQKEIDRLNEVAKNLNESLIDLQELGKYEQYIKRSNG | |
| | LPGPIGPPGPRGRTGDAGPVGPPGPPGPPGPPGPPSAGFDFSFLPQPPQEKAHDGGRYYR | |
| | ANDANVVRDRDLEVDTTLKSLSQQIENIRSPEGSRKNPARTCRDLKMCHSDWKSGEYWID | |
| | PNQGCNLDAIKVFCNMETGETCVYPTQPSVAQKNWYISKNPKDKRHVWFGESMTDGFQFE | |
| | YGGQGSDPADVAIQLTFLRLMSTEASQNITYHCKNSVAYMDQQTGNLKKALLLQGSNEIE | |
| | IRAEGNSRFTYSVTVDGCTSHTGAWGKTVIEYKTTKTSRLPIIDVAPLDVGAPDQEFGFD | |
| | VGPVCFL | |
| 21 | QCVNLTTRTQLPPAYTNSFTRGVYYPDKVFRSSVLHSTQDLFLPFFSNVTWFHAISGTNG | B.1.1.7 UK |
| | TKRFDNPVLPFNDGVYFASTEKSNIIRGWIFGTTLDSKTQSLLIVNNATNVVIKVCEFQF | variant |
| | CNDPFLGVYYHKNNKSWMESEFRVYSSANNCTFEYVSQPFLMDLEGKQGNFKNLREFVFK | SARS-CoV-2 |
| | NIDGYFKIYSKHTPINLVRDLPQGFSALEPLVDLPIGINITRFQTLLALHRSYLTPGDSS | spike S- |
| | SGWTAGAAAYYVGYLQPRTFLLKYNENGTITDAVDCALDPLSETKCTLKSFTVEKGIYQT | Trimer |
| | SNFRVQPTESIVRFPNITNLCPFGEVFNATRFASVYAWNRKRISNCVADYSVLYNSASFS | fusion |
| | TFKCYGVSPTKLNDLCFTNVYADSFVIRGDEVRQIAPGQTGKIADYNYKLPDDFTGCVIA | polypeptide |
| | WNSNNLDSKVGGNYNYLYRLFRKSNLKPFERDISTEIYQAGSTPCNGVEGFNCYFPLQSY | without |
| | GFQPTYGVGYQPYRVVVLSFELLHAPATVCGPKKSTNLVKNKCVNFNFNGLTGTGVLTES | signal |
| | NKKFLPFQQFGRDIADTTDAVRDPQTLEILDITPCSFGGVSVITPGTNTSNQVAVLYQGV | peptide, |
| | NCTEVPVAIHADQLTPTWRVYSTGSNVFQTRAGCLIGAEHVNNSYECDIPIGAGICASYQ | 1507 aa, |
| | TQTNSHRRAASVASQSIIAYTMSLGAENSVAYSNNSIAIPTNFTISVTTEILPVSMTKTS | S1/S2 furin |
| | VDCTMYICGDSTECSNLLLQYGSFCTQLNRALTGIAVEQDKNTQEVFAQVKQIYKTPPIK | cleavage |
| | DFGGFNFSQILPDPSKPSKRSFIEDLLFNKVTLADAGFIKQYGDCLGDIAARDLICAQKF | site 1 and |
| | NGLTVLPPLLTDEMIAQYTSALLAGTITSGWTFGAGAALQIPFAMQMAYRFNGIGVTQNV | proline |
| | LYENQKLIANQFNSAIGKIQDSLSSTASALGKLQDVVNQNAQALNTLVKQLSSNFGAISS | mutant |
| | VLNDILSRLDPPEAEVQIDRLITGRLQSLQTYVTQQLIRAAEIRASANLAATKMSECVLG | (685R→685A, |
| | QSKRVDFCGKGYHLMSFPQSAPHGVVFLHVTYVPAQEKNFTTAPAICHDGKAHFPREGVF | 986K/987V→ |
| | VSNGTHWFVTQRNFYEPQIITTDNTFVSGNCDVVIGIVNNTVYDPLQPELDSFKEELDKY | 986P/987P) |
| | FKNHTSPDVDLGDISGINASVVNIQKEIDRLNEVAKNLNESLIDLQELGKYEQYIKRSNG | |
| | LPGPIGPPGPRGRTGDAGPVGPPGPPGPPGPPGPPSAGFDFSFLPQPPQEKAHDGGRYYR | |
| | ANDANVVRDRDLEVDTTLKSLSQQIENIRSPEGSRKNPARTCRDLKMCHSDWKSGEYWID | |
| | PNQGCNLDAIKVFCNMETGETCVYPTQPSVAQKNWYISKNPKDKRHVWFGESMIDGFQFE | |
| | YGGQGSDPADVAIQLTFLRLMSTEASQNITYHCKNSVAYMDQQTGNLKKALLLQGSNEIE | |
| | IRAEGNSRFTYSVTVDGCTSHTGAWGKTVIEYKTTKTSRLPIIDVAPLDVGAPDQEFGFD | |
| | VGPVCFL | |
| 22 | QCVNLTTRTQLPPAYTNSFTRGVYYPDKVFRSSVLHSTQDLFLPFFSNVTWFHAIHVSGT | D614G |
| | NGTKRFDNPVLPFNDGVYFASTEKSNIIRGWIFGTTLDSKTQSLLIVNNATNVVIKVCEF | variant |
| | QFCNDPFLGVYYHKNNKSWMESEFRVYSSANNCTFEYVSQPFLMDLEGKQGNFKNLREFV | SARS-CoV-2 |
| | FKNIDGYFKIYSKHTPINLVRDLPQGFSALEPLVDLPIGINITRFQTLLALHRSYLTPGD | spike S- |
| | SSSGWTAGAAAYYVGYLQPRTFLLKYNENGTITDAVDCALDPLSETKCTLKSFTVEKGIY | Trimer |
| | OTSNFRVQPTESIVRFPNITNLCPFGEVFNATRFASVYAWNRKRISNCVADYSVLYNSAS | fusion |
| | FSTFKCYGVSPTKLNDLCFTNVYADSFVIRGDEVRQIAPGQTGKIADYNYKLPDDFTGCV | polypeptide |
| | IAWNSNNLDSKVGGNYNYLYRLFRKSNLKPFERDISTEIYQAGSTPCNGVEGFNCYFPLQ | without |
| | SYGFQPTNGVGYQPYRVVVLSFELLHAPATVCGPKKSTNLVKNKCVNFNFNGLTGTGVLT | signal |
| | ESNKKFLPFQQFGRDIADTTDAVRDPQTLEILDITPCSFGGVSVTTPGTNTSNQVAVLYQ | peptide, |
| | GVNCTEVPVAIHADQLTPTWRVYSTGSNVFQTRAGCLIGAEHVNNSYECDIPIGAGICAS | 1509 aa |
| | YQTQTNSPRRARSVASQSIIAYTMSLGAENSVAYSNNSIAIPTNFTISVTTEILPVSMTK | |
| | TSVDCTMYICGDSTECSNLLLQYGSFCTQLNRALTGIAVEQDKNTQEVFAQVKQIYKTPP | |
| | IKDFGGFNFSQILPDPSKPSKRSFIEDLLFNKVTLADAGFIKQYGDCLGDIAARDLICAQ | |
| | KFNGLTVLPPLLTDEMIAQYTSALLAGTITSGWTFGAGAALQIPFAMQMAYRFNGIGVTQ | |
| | NVLYENQKLIANQFNSAIGKIQDSLSSTASALGKLQDVVNQNAQALNTLVKQLSSNFGAI | |
| | SSVLNDILSRLDKVEAEVQIDRLITGRLQSLQTYVTQQLIRAAEIRASANLAATKMSECV | |
| | LGQSKRVDFCGKGYHLMSFPQSAPHGVVFLHVTYVPAQEKNFTTAPAICHDGKAHFPREG | |
| | VFVSNGTHWFVTQRNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQYIKRS | |
| | NGLPGPIGPPGPRGRTGDAGPVGPPGPPGPPGPPGPPSAGFDFSFLPQPPQEKAHDGGRY | |
| | YRANDANVVRDRDLEVDTTLKSLSQQIENIRSPEGSRKNPARTCRDLKMCHSDWKSGEYW | |
| | IDPNQGCNLDAIKVFCNMETGETCVYPTQPSVAQKNWYISKNPKDKRHVWFGESMTDGFQ | |
| | FEYGGQGSDPADVAIQLTFLRLMSTEASQNITYHCKNSVAYMDQQTGNLKKALLLQGSNE | |
| | IEIRAEGNSRFTYSVTVDGCTSHTGAWGKTVIEYKTTKTSRLPIIDVAPLDVGAPDQEFG | |
| | FDVGPVCFL | |
| 23 | QCVNLTTRTQLPPAYTNSFTRGVYYPDKVFRSSVLHSTQDLFLPFFSNVTWFHAIHVSGI | D614G |
| | NGTKRFDNPVLPFNDGVYFASTEKSNIIRGWIFGTTLDSKTQSLLIVNNATNVVIKVCEF | variant |
| | QFCNDPFLGVYYHKNNKSWMESEFRVYSSANNCTFEYVSQPFLMDLEGKQGNFKNLREFV | SARS-CoV-2 |
| | FKNIDGYFKIYSKHTPINLVRDLPQGFSALEPLVDLPIGINTTRFQTLLALHRSYLTPGD | spike S- |
| | SSSGWTAGAAAYYVGYLQPRTFLLKYNENGTITDAVDCALDPLSETKCTLKSFTVEKGIY | Trimer |
| | QTSNFRVQPTESIVRFPNITNLCPFGEVFNATRFASVYAWNRKRISNCVADYSVLYNSAS | fusion |
| | FSTFKCYGVSPTKLNDLCFTNVYADSFVIRGDEVRQIAPGQTGKIADYNYKLPDDFTGCV | polypeptide |
| | IAWNSNNLDSKVGGNYNYLYRLFRKSNLKPFERDISTEIYQAGSTPCNGVEGFNCYFPLQ | without |
| | SYGFQPTNGVGYQPYRVVVLSFELLHAPATVCGPKKSTNLVKNKCVNFNFNGLTGTGVLI | signal |
| | ESNKKFLPFQQFGRDIADTTDAVRDPQILEILDITPCSFGGVSVITPGTNTSNQVAVLYQ | peptide, |
| | GVNCTEVPVAIHADQLTPTWRVYSTGSNVFQTRAGCLIGAEHVNNSYECDIPIGAGICAS | 1509 aa, |
| | YQTQTNSPRRAASVASQSIIAYTMSLGAENSVAYSNNSIAIPINFTISVTTEILPVSMTK | S1/S2 furin |
| | TSVDCTMYICGDSTECSNLLLQYGSFCTQLNRALTGIAVEQDKNTQEVFAQVKQIYKTPP | cleavage |
| | IKDFGGFNFSQILPDPSKPSKRSFIEDLLFNKVTLADAGFIKQYGDCLGDIAARDLICAQ | site 1 |
| | KFNGLTVLPPLLTDEMIAQYTSALLAGTITSGWTFGAGAALQIPFAMQMAYRFNGIGVTQ | mutant |
| | NVLYENQKLIANQFNSAIGKIQDSLSSTASALGKLQDVVNQNAQALNTLVKQLSSNFGAI | (685R→685A) |
| | SSVLNDILSRLDKVEAEVQIDRLITGRLQSLQTYVTQQLIRAAEIRASANLAATKMSECV | |
| | LGQSKRVDFCGKGYHLMSFPQSAPHGVVFLHVTYVPAQEKNFTTAPAICHDGKAHFPREG | |
| | VFVSNGTHWFVTQRNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQYIKRS | |
| | NGLPGPIGPPGPRGRTGDAGPVGPPGPPGPPGPPGPPSAGFDFSFLPQPPQEKAHDGGRY | |
| | YRANDANVVRDRDLEVDTTLKSLSQQIENIRSPEGSRKNPARTCRDLKMCHSDWKSGEYW | |
| | IDPNQGCNLDAIKVFCNMETGETCVYPTOPSVAQKNWYISKNPKDKRHVWFGESMTDGFQ | |
| | FEYGGQGSDPADVAIQLTFLRLMSTEASQNITYHCKNSVAYMDQQTGNLKKALLLOGSNE | |
| | IEIRAEGNSRFTYSVTVDGCTSHTGAWGKTVIEYKTTKTSRLPIIDVAPLDVGAPDQEFG | |
| | FDVGPVCFL | |
| 24 | QCVNLTTRTQLPPAYTNSFTRGVYYPDKVFRSSVLHSTQDLFLPFFSNVTWFHAIHVSGT | D614G |
| | NGTKRFDNPVLPFNDGVYFASTEKSNIIRGWIFGTTLDSKTQSLLIVNNATNVVIKVCEF | variant |
| | QFCNDPFLGVYYHKNNKSWMESEFRVYSSANNCTFEYVSQPFLMDLEGKQGNFKNLREFV | SARS-CoV-2 |
| | FKNIDGYFKIYSKHTPINLVRDLPQGFSALEPLVDLPIGINITRFQTLLALHRSYLTPGD | spike S- |
| | SSSGWTAGAAAYYVGYLQPRTFLLKYNENGTITDAVDCALDPLSETKCTLKSFTVEKGIY | Trimer |
| | QTSNFRVQPTESIVRFPNITNLCPFGEVFNATRFASVYAWNRKRISNCVADYSVLYNSAS | fusion |
| | FSTFKCYGVSPTKLNDLCFTNVYADSFVIRGDEVRQIAPGQTGKIADYNYKLPDDFTGCV | polypeptide |
| | IAWNSNNLDSKVGGNYNYLYRLFRKSNLKPFERDISTEIYQAGSTPCNGVEGFNCYFPLQ | without |
| | SYGFQPTNGVGYOPYRVVVLSFELLHAPATVCGPKKSTNLVKNKCVNFNFNGLIGTGVLT | signal |
| | ESNKKFLPFQQFGRDIADTTDAVRDPQTLEILDITPCSFGGVSVITPGTNTSNQVAVLYQ | peptide, |
| | GVNCTEVPVAIHADQLTPTWRVYSTGSNVFQTRAGCLIGAEHVNNSYECDIPIGAGICAS | 1509 aa, |
| | YQTQTNSPRRARSVASQSIIAYTMSLGAENSVAYSNNSIAIPTNFTISVTTEILPVSMTK | proline |
| | TSVDCTMYICGDSTECSNLLLQYGSFCTQLNRALTGIAVEQDKNTQEVFAQVKQIYKTPP | mutant |
| | IKDFGGFNFSQILPDPSKPSKRSFIEDLLFNKVTLADAGFTKQYGDCLGDIAARDLICAQ | (986K/987V→ |
| | KFNGLTVLPPLLIDEMIAQYTSALLAGTITSGWTFGAGAALQIPFAMQMAYRFNGIGVTQ | 986P/987P) |
| | NVLYENQKLIANQFNSAIGKIQDSLSSTASALGKLQDVVNQNAQALNTLVKQLSSNFGAI | |
| | SSVLNDILSRLDPPEAEVQIDRLITGRLQSLQTYVTQQLIRAAEIRASANLAATKMSECV | |
| | LGQSKRVDFCGKGYHLMSFPQSAPHGVVFLHVTYVPAQEKNFTTAPAICHDGKAHFPREG | |
| | VFVSNGTHWFVTQRNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQYIKRS | |
| | NGLPGPIGPPGPRGRTGDAGPVGPPGPPGPPGPPGPPSAGFDFSFLPQPPQEKAHDGGRY | |
| | YRANDANVVRDRDLEVDTTLKSLSQQIENIRSPEGSRKNPARTCRDLKMCHSDWKSGEYW | |
| | IDPNQGCNLDAIKVFCNMETGETCVYPTQPSVAQKNWYISKNPKDKRHVWFGESMTDGFQ | |
| | FEYGGQGSDPADVAIQLTFLRLMSTEASQNITYHCKNSVAYMDQQTGNLKKALLLQGSNE | |
| | IEIRAEGNSRFTYSVTVDGCTSHTGAWGKTVIEYKTTKTSRLPIIDVAPLDVGAPDQEFG | |
| | FDVGPVCFL | |
| 25 | QCVNLTTRTQLPPAYTNSFTRGVYYPDKVFRSSVLASTQDLFLPFFSNVTWFHAIHVSGT | D614G |
| | NGTKRFDNPVLPFNDGVYFASTEKSNIIRGWIFGTTLDSKTQSLLIVNNATNVVIKVCEE | variant |
| | QFCNDPFLGVYYHKNNKSWMESEFRVYSSANNCTFEYVSQPFLMDLEGKQGNFKNLREFV | SARS-CoV-2 |
| | FKNIDGYFKIYSKHTPINLVRDLPQGFSALEPLVDLPIGINITRFQTLLALHRSYLTPGD | spike S- |
| | SSSGWTAGAAAYYVGYLQPRTFLLKYNENGTITDAVDCALDPLSETKCTLKSFTVEKGIY | Trimer |
| | QTSNFRVQPTESIVRFPNITNLCPFGEVFNATRFASVYAWNRKRISNCVADYSVLYNSAS | rus1On |
| | FSTFKCYGVSPTKLNDLCFTNVYADSFVIRGDEVRQIAPGQTGKIADYNYKLPDDFTGCV | polypeptide |
| | lAWNSNNLDSKVGGNYNYLYRLFRKSNLKPFERDISTEIYQAGSTPCNGVEGFNCYFPLQ | without |
| | SYGFQPTNGVGYQPYRVVVLSFELLHAPATVCGPKKSTNLVKNKCVNFNFNGLTGTGVLI | signal |
| | ESNKKFLPFQQFGRDIADTTDAVRDPQTLEILDITPCSFGGVSVITPGTNTSNQVAVLYQ | peptide, |
| | GVNCTEVPVAIHADQLTPTWRVYSTGSNVFQTRAGCLIGAEHVNNSYECDIPIGAGICAS | 1509 aa, |
| | YQTQINSPRRAASVASQSIIAYTMSLGAENSVAYSNNSIAIPTNFTISVTTEILPVSMTK | S1/S2 furin |
| | TSVDCTMYICGDSTECSNLLLQYGSFCTQLNRALTGIAVEQDKNTQEVFAQVKQIYKTPP | cleavage |
| | IKDFGGFNFSQILPDPSKPSKRSFIEDLLFNKVTLADAGFIKQYGDCLGDIAARDLICAQ | site 1 and |
| | KFNGLTVLPPLLTDEMIAQYTSALLAGTITSGWTFGAGAALQIPFAMQMAYRFNGIGVTQ | proline |
| | NVLYENQKLIANQFNSAIGKIQDSLSSTASALGKLQDVVNQNAQALNTLVKQLSSNFGAI | mutant |
| | SSVLNDILSRLDPPEAEVQIDRLITGRLQSLQTYVTQQLIRAAEIRASANLAATKMSECV | (685R→685A, |
| | LGQSKRVDFCGKGYHLMSFPQSAPHGVVFLHVTYVPAQEKNFTTAPAICHDGKAHFPREG | 986K/987V→ |
| | VFVSNGTHWFVTQRNFYEPQIITTDNTFVSGNCDVVTGIVNNTVYDPLQPELDSFKEELD | 986P/987P) |
| | KYFKNHTSPDVDLGDISGINASVVNIQKEIDRLNEVAKNLNESLIDLQELGKYEQYIKRS | |
| | NGLPGPIGPPGPRGRTGDAGPVGPPGPPGPPGPPGPPSAGFDFSFLPQPPQEKAHDGGRY | |
| | YRANDANVVRDRDLEVDTTLKSLSQQIENIRSPEGSRKNPARTCRDLKMCHSDWKSGEYW | |
| | IDPNQGCNLDAIKVFCNMETGETCVYPTOPSVAQKNWYISKNPKDKRHVWFGESMTDGFQ | |
| | FEYGGOGSDPADVAIQLTFLRLMSTEASQNITYHCKNSVAYMDQQTGNLKKALLLQGSNE | |
| | IEIRAEGNSRFTYSVTVDGCTSHIGAWGKTVIEYKTTKTSRLPIIDVAPLDVGAPDQEFG | |
| | FDVGPVCFL | |
| 26 | SDLDRCTTFDDVQAPNYTQHTSSMRGVYYPDEIFRSDTLYLTQDLFLPFYSNVTGFHTIN | SARS-CoV-1 |
| | HTFDNPVIPFKDGIYFAATEKSNVVRGWVFGSTMNNKSQSVIIINNSTNVVIRACNFELC | spike S- |
| | DNPFFAVSKPMGTQTHTMIFDNAFNCTFEYISDAFSLDVSEKSGNFKHLREFVFKNKDGF | Trimer |
| | LYVYKGYQPIDVVRDLPSGFNTLKPIFKLPLGINITNFRAILTAFLPAQDTWGTSAAAYF | fusion |
| | VGYLAPLIFMLKYDENGTITDAVDCSQNPLAELKCSVKSFEIDKGIYQTSNFRVVPSRDV | polypeptide |
| | VRFPNIINLCPFGEVFNATKFPSVYAWERKRISNCVADYSVLYNSTFFSTFKCYGVSATK | without |
| | LNDLCFSNVYADSFVVKGDDVRQIAPGQTGVIADYNYKLPDDFMGCVLAWNTRNIDATST | signal |
| | GNYNYKYRYLRHGKLRPFERDISNVPFSPDGKPCTPPALNCYWPLNDYGFYTTTGIGYQP | peptide, |
| | YRVVVLSFELLNAPATVCGPKLSTDLIKNQCVNFNFNGLTGTGVLTPSSKRFQPFQQFGR | 1491 aa |
| | DVSDFTDSVRDPKTSEILDISPCSFGGVSVITPGTNASSEVAVLYQDVNCTDVSTAIHAD | |
| | QLTPAWRIYSTGNNVFQTQAGCLIGAEHVDTSYECDIPIGAGICASYHTVSLLRSTSQKS | |
| | IVAYTMSLGADSSIAYSNNTIAIPTNFSISITTEVMPVSMAKTSVDCNMYICGDSTECAN | |
| | LLLQYGSFCTQLNRALSGIAAEQDRNTREVFAQVKQMYKTPTLKDFGGFNFSQILPDPLK | |
| | PTKRSFIEDLLFNKVTLADAGFMKQYGECLGDINARDLICAQKFNGLTVLPPLLTDDMIA | |
| | AYTAALVSGTATAGWTFGAGAALQIPFAMQMAYRFNGIGVTQNVLYENQKQIANQFNKAI | |
| | SQIQESLTTTSTALGKLQDVVNQNAQALNTLVKQLSSNFGAISSVLNDILSRLDKVEAEV | |
| | QIDRLITGRLQSLQTYVTQQLIRAAEIRASANLAATKMSECVLGQSKRVDFCGKGYHLMS | |
| | FPQAAPHGVVFLHVTYVPSQERNFTTAPAICHEGKAYFPREGVFVFNGTSWFITQRNFFS | |
| | PQIITTDNTFVSGNCDVVIGIINNTVYDPLQPELDSFKEELDKYFKNHTSPDVDLGDISG | |
| | INASVVNIQEEIDRLNEVAKNLNESLIDLQELGKYEQYIKRSNGLPGPIGPPGPRGRTGD | |
| | AGPVGPPGPPGPPGPPGPPSAGFDFSFLPQPPQEKAHDGGRYYRANDANVVRDRDLEVDT | |
| | TLKSLSQQIENIRSPEGSRKNPARTCRDLKMCHSDWKSGEYWIDPNQGCNLDAIKVFCNM | |
| | ETGETCVYPTQPSVAQKNWYISKNPKDKRHVWFGESMTDGFQFEYGGQGSDPADVAIQLT | |
| | FLRLMSTEASQNITYHCKNSVAYMDQQTGNLKKALLLQGSNEIEIRAEGNSRFTYSVTVD | |
| | GCTSHTGAWGKTVIEYKTTKTSRLPIIDVAPLDVGAPDQEFGFDVGPVCFL | |
| 27 | QCVNLTTRTQLPPAYTNSFTRGVYYPDKVFRSSVLHSTQDLFLPFFSNVTWFHAIHVSGT | Prototypic |
| | NGTKRFDNPVLPFNDGVYFASTEKSNIIRGWIFGTTLDSKTQSLLIVNNATNVVIKVCEF | SARS-CoV-2 |
| | QFCNDPFLGVYYHKNNKSWMESEFRVYSSANNCTFEYVSQPFLMDLEGKQGNFKNLREFV | spike |
| | FKNIDGYFKIYSKHTPINLVRDLPQGFSALEPLVDLPIGINITRFQTLLALHRSYLTPGD | protein |
| | SSSGWTAGAAAYYVGYLQPRTFLLKYNENGTITDAVDCALDPLSETKCTLKSFTVEKGIY | ectodomain |
| | QTSNFRVQPTESIVRFPNITNLCPFGEVFNATRFASVYAWNRKRISNCVADYSVLYNSAS | signal |
| | FSTFKCYGVSPTKLNDLCFTNVYADSFVIRGDEVRQIAPGQTGKIADYNYKLPDDFTGCV | peptide |
| | IAWNSNNLDSKVGGNYNYLYRLFRKSNLKPFERDISTEIYQAGSTPCNGVEGFNCYFPLQ | |
| | SYGFQPTNGVGYQPYRVVVLSFELLHAPATVCGPKKSTNLVKNKCVNFNFNGLTGTGVLT | |
| | ESNKKFLPFQQFGRDIADTTDAVRDPQTLEILDITPCSFGGVSVITPGTNTSNQVAVLYQ | |
| | DVNCTEVPVAIHADQLTPTWRVYSTGSNVFQTRAGCLIGAEHVNNSYECDIPIGAGICAS | |
| | YQTQTNSPRRARSVASQSIIAYTMSLGAENSVAYSNNSIAIPTNFTISVTTEILPVSMTK | |
| | TSVDCTMYICGDSTECSNLLLQYGSFCTQLNRALTGIAVEQDKNTQEVFAQVKQIYKIPP | |
| | IKDFGGFNFSQILPDPSKPSKRSFIEDLLFNKVTLADAGFIKQYGDCLGDIAARDLICAQ | |
| | KFNGLTVLPPLLTDEMIAQYTSALLAGTITSGWIHGAGAALQIPFAMQMAYRFNGIGVTQ | |
| | NVLYENQKLIANQFNSAIGKIQDSLSSTASALGKLQDVVNQNAQALNTLVKQLSSNFGAI | |
| | SSVLKDILSRLDKVEAEVQIDBLITGRLQSLQTYVTQQLIHAAEIRASAXLAATKWSECV | |
| | LGQSKRVDFCGKGYHLMSFPQSAPHGVVFLHVTYVPAQEKNFTTAPAICHDGKAHFPREG | |
| | VFVSNGTHWFVTORNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQ | |
| 28 | QCVNLTTRTQLPPAYTNSFTRGVYYPDKVFRSSVLHSTQDLFLPFFSNVTWFHAIHVSGT | Prototypic |
| | NGTKRFDNPVLPFNDGVYFASTEKSNIIRGWIFGTTLDSKTQSLLIVNNATNVVIKVCEF | SARS-CoV-2 |
| | QFCNDPFLGVYYHKNNKSWMESEFRVYSSANNCTFEYVSQPFLMDLEGKQGNFKNLREFV | spike |
| | FKNIDGYFKIYSKHTPINLVRDLPQGFSALEPLVDLPIGINITRFQTLLALHRSYLTPGD | protein |
| | SSSGWTAGAAAYYVGYLQPRTFLLKYNENGTITDAVDCALDPLSETKCTLKSFTVEKGIY | ectodomain |
| | QTSNFRVQPTESIVRFPNITNLCPFGEVFNATRFASVYAWNRKRISNCVADYSVLYNSAS | without |
| | FSTFKCYGVSPTKLNDLCFTNVYADSFVIRGDEVRQIAPGQTGKIADYNYKLPDDFTGCV | signal |
| | IAWNSNNLDSKVGGNYNYLYRLFRKSNLKPFERDISTEIYQAGSTPCNGVEGFNCYFPLQ | peptide, |
| | SYGFQPTNGVGYQPYRVVVLSFELLHAPATVCGPKKSTNLVKNKCVNFNFNGLTGTGVLT | S1/S2 furin |
| | ESNKKFLPFQQFGRDIADTTDAVRDPQTLEILDITPCSFGGVSVITPGTNTSNQVAVLYQ | cleavage |
| | DVNCTEVPVAIHADQLTPTWRVYSTGSNVFQTRAGCLIGAEHVNNSYECDIPIGAGICAS | site 1 |
| | YQTQTNSPRRAASVASQSIIAYTMSLGAENSVAYSNNSIAIPTNFTISVTTEILPVSMTK | mutant |
| | TSVDCTMYICGDSTECSNLLLQYGSFCTQLNRALTGIAVEQDKNTQEVFAQVKQIYKIPP | (685R→685A) |
| | IKDFGGFNFSQILPDPSKPSKRSFIEDLLFNKVTLADAGFIKQYGDCLGDIAARDLICAQ | |
| | KFNGLTVLPPLLTDEMIAQYTSALLAGTITSGWTFGAGAALQIPFAMQMAYRFNGIGVTQ | |
| | NVLYENQKLIANQFNSAIGKIQDSLSSTASALGKLQDVVNQNAQALNTLVKQLSSNFGAI | |
| | SSVLNDILSRLDKVEAEVQIDRLITGRLQSLQTYVTQQLIRAAEIRASANLAATKMSECV | |
| | LGQSKRVDFCGKGYHLMSFPQSAPHGVVFLHVTYVPAQEKNFTTAPAICHDGKAHFPREG | |
| | VFVSNGTHWFVTORNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQ | |
| 29 | QCVNLTTRTQLPPAYTNSFTRGVYYPDKVFRSSVLHSTQDLFLPFFSNVTWFHAIHVSGT | Prototypic |
| | NGTKRFDNPVLPFNDGVYFASTEKSNIIRGWIFGTTLDSKTQSLLIVNNATNVVIKVCEF | SARS-CoV-2 |
| | QFCNDPFLGVYYHKNNKSWMESEFRVYSSANNCTFEYVSQPFLMDLEGKQGNFKNLREFV | spike |
| | FKNIDGYFKIYSKHTPINLVRDLPQGFSALEPLVDLPIGINITRFQTLLALHRSYLTPGD | protein |
| | SSSGWTAGAAAYYVGYLQPRTFLLKYNENGTITDAVDCALDPLSETKCTLKSFTVEKGIY | ectodomain |
| | QTSNFRVOPTESIVRFPNITNLCPFGEVFNATRFASVYAWNRKRISNCVADYSVLYNSAS | without |
| | FSTFKCYGVSPTKLNDLCFTNVYADSFVIRGDEVRQIAPGQTGKIADYNYKLPDDFTGCV | signal |
| | IAWNSNNLDSKVGGNYNYLYRLFRKSNLKPFERDISTEIYQAGSTPCNGVEGFNCYFPLQ | peptide, |
| | SYGFQPTNGVGYOPYRVVVLSFELLHAPATVCGPKKSTNLVKNKCVNFNFNGLIGTGVLT | proline |
| | ESNKKFLPFQQFGRDIADTTDAVRDPQTLEILDITPCSFGGVSVITPGTNTSNQVAVLYQ | mutant |
| | DVNCTEVPVAIHADQLTPTWRVYSTGSNVFQTRAGCLIGAEHVNNSYECDIPIGAGICAS | (986K/987V→ |
| | YQTQTNSPRRARSVASQSIIAYTMSLGAENSVAYSNNSIAIPTNFTISVTTEILPVSMTK | 986P/987P) |
| | TSVDCTMYICGDSTECSNLLLQYGSFCTQLNRALTGIAVEQDKNTQEVFAQVKQIYKTPP | |
| | IKDFGGFNFSQILPDPSKPSKRSFIEDLLFNKVTLADAGFTKQYGDCLGDIAARDLICAQ | |
| | KFNGLTVLPPLLTDEMIAQYTSALLAGTITSGWTFGAGAALQIPFAMQMAYRFNGIGVTQ | |
| | NVLYENQKLIANQFNSAIGKIQDSLSSTASALGKLQDVVNQNAQALNTLVKQLSSNFGAI | |
| | SSVLNDILSRLDPPEAEVQIDRLITGRLQSLQTYVTQQLIRAAEIRASANLAATKMSECV | |
| | LGQSKRVDFCGKGYHLMSFPQSAPHGVVFLHVTYVPAQEKNFTTAPAICHDGKAHFPREG | |
| | VFVSNGTHWFVTQRNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQ | |
| 30 | QCVNLTTRTQLPPAYTNSFTRGVYYPDKVFRSSVLHSTQDLFLPFFSNVTWFHAIHVSGT | Prototypic |
| | NGTKRFDNPVLPFNDGVYFASTEKSNIIRGWIFGTTLDSKTQSLLIVNNATNVVIKVCEE | SARS-CoV-2 |
| | QFCNDPFLGVYYHKNNKSWMESEFRVYSSANNCTFEYVSQPFLMDLEGKQGNFKNLREFV | spike |
| | FKNIDGYFKIYSKHTPINLVRDLPQGFSALEPLVDLPIGINITRFQTLLALHRSYLTPGD | protein |
| | SSSGWTAGAAAYYVGYLQPRTFLLKYNENGTITDAVDCALDPLSETKCTLKSFTVEKGIY | ectodomain |
| | QTSNFRVQPTESIVRFPNITNLCPFGEVFNATRFASVYAWNRKRISNCVADYSVLYNSAS | without |
| | FSTFKCYGVSPTKLNDLCFTNVYADSFVIRGDEVRQIAPGQTGKIADYNYKLPDDFTGCV | signal |
| | IAWNSNNLDSKVGGNYNYLYRLFRKSNLKPFERDISTEIYQAGSTPCNGVEGFNCYFPLQ | peptide, |
| | SYGFQPTNGVGYQPYRVVVLSFELLHAPATVCGPKKSTNLVKNKCVNFNFNGLTGTGVLI | S1/S2 furin |
| | ESNKKFLPFQQFGRDIADTTDAVRDPQTLEILDITPCSFGGVSVITPGTNTSNQVAVLYQ | cleavage |
| | DVNCTEVPVAIHADQLTPTWRVYSTGSNVFQTRAGCLIGAEHVNNSYECDIPIGAGICAS | site 1 and |
| | YQTQTNSPRRAASVASOSIIAYTMSLGAENSVAYSNNSIAIPTNFTISVTTEILPVSMTK | proline |
| | TSVDCTMYICGDSTECSNLLLQYGSFCTQLNRALTGIAVEQDKNTQEVFAQVKQIYKTPP | mutant |
| | IKDFGGFNFSQILPDPSKPSKRSFIEDLLFNKVTLADAGFIKQYGDCLGDIAARDLICAQ | (685R→685A, |
| | KFNGLTVLPPLLTDEMIAQYTSALLAGTITSGWTFGAGAALQIPFAMQMAYRFNGIGVTQ | 986K/987V→ |
| | NVLYENQKLIANQFNSAIGKIQDSLSSTASALGKLQDVVNQNAQALNTLVKQLSSNFGAI | 986P/987P) |
| | SSVLNDILSRLDPPEAEVQIDRLITGRLQSLQTYVTQQLIRAAEIRASANLAATKMSECV | |
| | LGQSKRVDFCGKGYHLMSFPQSAPHGVVFLHVTYVPAQEKNFTTAPAICHDGKAHFPREG | |
| | VFVSNGTHWFVTQRNFYEPQIITTDNTFVSGNCDWTGIVNNTVYDPLQPELDSFKEELD | |
| | KYFKNHTSPDVDLGDISGINASVVNIQKEIDRLNEVAKNLNESLIDLQELGKYEQ | |
| 31 | QCVNLTTRTQLPPAYTNSFTRGVYYPDKVFRSSVLHSTQLLFLPFFSNVTWFHAIHVSGT | Prototypic |
| | NGTKRFDNPVLPFNDGVYFASTEKSNIIRGWIFGTTLDSKTQSLLIVNNATNVVIKVCEF | SARS-CoV-2 |
| | QFCNDPFLGVYYHKNNKSWMESEFRVYSSANNCTFEYVSQPFLMDLEGKQGNFKNLREFV | spike |
| | FKNIDGYFKIYSKHTPINLVRDLPQGFSALEPLVDLPIGINITRFQTLLALHRSYLTPGD | protein |
| | SSSGWTAGAAAYYVGYLQPRTFLLKYNENGTITDAVDCALDPLSETKCTLKSFTVEKGIY | NTD/RBD |
| | QTSNFRVOPTESIVRFPNITNLCPFGEVFNATRFASVYAWNRKRISNCVADYSVLYNSAS | fragment |
| | FSTFKCYGVSPTKLNDLCFTNVYADSFVIRGDEVRQIAPGQTGKIADYNYKLPDDFTGCV | without |
| | IAWNSNNLDSKVGGNYNYLYRLFRKSNLKPFERDISTEIYQAGSTPCNGVEGFNCYFPLQ | signal |
| | SYGFQPTNGVGYOPYRVVVLSFELLHAPATVCGPKKSTNLVKNKC | peptide |
| 32 | QCVNLTTRTQLPPAYTNSFTRGVYYPDKVFRSSVLHSTQDLFLPFFSNVTWFHAIHVSGI | Prototypic |
| | NGTKRFDNPVLPFNDGVYFASTEKSNIIRGWIFGTTLDSKTQSLLIVNNATNVVIKVCEF | SARS-CoV-2 |
| | QFCNDPFLGVYYHKNNKSWMESEFRVYSSANNCTFEYVSQPFLMDLEGKQGNFKNLREFV | spike |
| | FKNIDGYFKIYSKHTPINLVRDLPQGFSALEPLVDLPIGINITRFQTLLALHRSYLTPGD | protein S1 |
| | SSSGWTAGAAAYYVGYLQPRTFLLKYNENGTITDAVDCALDPLSETKCTLKSFTVEKGIY | fragment |
| | QTSNFRVQPTESIVRFPNITNLCPFGEVFNATRFASVYAWNRKRISNCVADYSVLYNSAS | without |
| | FSTFKCYGVSPTKLNDLCFTNVYADSFVIRGDEVRQIAPGQTGKIADYNYKLPDDFTGCV | signal |
| | IAWNSNNLDSKVGGNYNYLYRLFRKSNLKPFERDISTEIYQAGSTPCNGVEGFNCYFPLQ | peptide |
| | SYGFQPTNGVGYOPYRVVVLSFELLHAPATVCGPKKSTNLVKNKCVNFNFNGLIGTGVLT | |
| | ESNKKFLPFQQFGRDIADTTDAVRDPQTLEILDITPCSFGGVSVITPGTNTSNQVAVLYQ | |
| | DVNCTEVPVAIHADQLTPTWRVYSTGSNVFQTRAGCLIGAEHVNNSYECDIPIGAGICAS | |
| | YQTQTNSP | |
| 33 | SVASQSIIAYTMSLGAENSVAYSNNSIAIPTNFTISVTTEILPVSMTKTSVDCIMYICGD | Prototypic |
| | STECSNLLLQYGSFCTQLNRALTGIAVEQDKNTQEVFAQVKQIYKTPPIKDFGGFNFSQI | SARS-CoV-2 |
| | LPDPSKPSKRSFIEDLLFNKVTLADAGFIKQYGDCLGDIAARDLICAQKFNGLTVLPPLL | spike |
| | TDEMIAQYTSALLAGTITSGWTFGAGAALQIPFAMQMAYRFNGIGVTQNVLYENQKLIAN | protein S2 |
| | QFNSAIGKIQDSLSSTASALGKLQDVVNQNAQALNTLVKQLSSNFGAISSVLNDILSRLD | fragment |
| | KVEAEVQIDRLITGRLQSLQTYVTQQLIRAAEIRASANLAATKMSECVLGQSKRVDFCGK | (cleaved at |
| | GYHLMSFPQSAPHGVVFLHVTYVPAQEKNFTTAPAICHDGKAHFPREGVFVSNGTHWFVT | S1/S2, site |
| | QRNFYEPQIITTDNTFVSGNCDVVIGIVNNTVYDPLQPELDSFKEELDKYFKNHTSPDVD | 1) |
| | LGDISGINASVVNIQKEIDRLNEVAKNLNESLIDLQELGKYEQ | |
| 34 | TMSLGAENSVAYSNNSIAIPTNFTISVTTEILPVSMTKTSVDCTMYICGDSTECSNLLLQ | Prototypic |
| | YGSFCTQLNRALTGIAVEQDKNTQEVFAQVKQIYKTPPIKDFGGFNFSQILPDPSKPSKR | SARS-CoV-2 |
| | SFIEDLLFNKVTLADAGFIKQYGDCLGDIAARDLICAQKFNGLTVLPPLLTDEMIAQYTS | spike |
| | ALLAGTITSGWTFGAGAALQIPFAMQMAYRFNGIGVTQNVLYENQKLIANQFNSAIGKIQ | protein S2 |
| | DSLSSTASALGKLQDVVNQNAQALNTLVKQLSSNFGAISSVLNDILSRLDKVEAEVQIDR | fragment |
| | LITGRLQSLQTYVTQQLIRAAEIRASANLAATKMSECVLGQSKRVDFCGKGYHLMSFPQS | (cleaved at |
| | APHGVVFLHVTYVPAQEKNFTTAPAICHDGKAHFPREGVFVSNGTHWFVTQRNFYEPQII | S1/S2, site |
| | TTDNTFVSGNCDVVIGIVNNTVYDPLQPELDSFKEELDKYFKNHTSPDVDLGDISGINAS | 2) |
| | VVNIQKEIDRLNEVAKNLNESLIDLQELGKYEQ | |
| 35 | SFIEDLLFNKVTLADAGFIKQYGDCLGDIAARDLICAQKFNGLTVLPPLLTDEMIAQYTS | Prototypic |
| | ALLAGTITSGWTFGAGAALQIPFAMQMAYRFNGIGVTQNVLYENQKLIANQFNSAIGKIQ | SARS-CoV-2 |
| | DSLSSTASALGKLQDVVXQNACALNTLVKQLSSNFGAISSVLXDILSRLDKVEAEVQIDR | spike |
| | LITGRLQSLQTYVTQQLIRAAEIRASANLAATKMSECVLGQSKRVDFCGKGYHLMSFPQS | protein 32 |
| | APHGVVFLHVTYVPAQEKNFTTAPAICHDGKAHFPREGVFVSNGTHWFVTORNFYEPQII | fragment |
| | TTDNTFVSGNCDVVIGIVNNTVYDPLQPELDSFKEELDKYFKNHTSPDVDLGDISGINAS | (cleaved at |
| | VVNIQKEIDRLNEVAKNLNESLIDLQELGKYEQ | S2′) |
| 36 | QCVNLTTRTQLPPAYTNSFTRGVYYPDKVFRSSVLHSTQDLFLPFFSNVTWFHAIHVSGT | B.1.351 |
| | NGTKRFDNPVLPFNDGVYFASTEKSNIIRGWIFGTTLDSKTQSLLIVNNATNVVIKVCEF | South |
| | QFCNDPFLGVYYHKNNKSWMESEFRVYSSANNCTFEYVSQPFLMDLEGKQGNFKNLREFV | African |
| | FKNIDGYFKIYSKHTPINLVRDLPQGFSALEPLVDLPIGINITRFQTLLALHRSYLTPGD | variant |
| | SSSGWTAGAAAYYVGYLQPRTFLLKYNENGTITDAVDCALDPLSETKCTLKSFTVEKGIY | SARS-CoV-2 |
| | QTSNFRVQPTESIVRFPNITNLCPFGEVFNATRFASVYAWNRKRISNCVADYSVLYNSAS | spike |
| | FSTFKCYGVSPTKLNDLCFTNVYADSFVIRGDEVRQIAPGQTGNIADYNYKLPDDFTGCV | protein |
| | IAWNSNNLDSKVGGNYNYLYRLFRKSNLKPFERDISTEIYQAGSTPCNGVKGFNCYFPLQ | ectodomain |
| | SYGFQPTYGVGYOPYRVVVLSFELLHAPATVCGPKKSTNLVKNKCVNFNFNGLIGTGVLT | without |
| | ESNKKFLPFQQFGRDIADTTDAVRDPQTLEILDITPCSFGGVSVITPGTNTSNQVAVLYQ | signal |
| | GVNCTEVPVAIHADQLTPTWRVYSTGSNVFQTRAGCLIGAEHVNNSYECDIPIGAGICAS | peptide |
| | YQTQTNSPRRARSVASQSIIAYTMSLGAENSVAYSNNSIAIPTNFTISVTTEILPVSMTK | |
| | TSVDCTMYICGDSTECSNLLLQYGSFCTQLNRALTGIAVEQDKNTQEVFAQVKQIYKTPP | |
| | IKDFGGFNFSQILPDPSKPSKRSFIEDLLFNKVTLADAGFTKQYGDCLGDIAARDLICAQ | |
| | KFNGLTVLPPLLIDEMIAQYTSALLAGTITSGWTFGAGAALQIPFAMQMAYRFNGIGVTQ | |
| | NVLYENQKLIANQFNSAIGKIQDSLSSTASALGKLQDVVNQNAQALNTLVKQLSSNFGAI | |
| | SSVLNDILSRLDKVEAEVQIDRLITGRLQSLQTYVTQQLIRAAEIRASANLAATKMSECV | |
| | LGQSKRVDFCGKGYHLMSFPQSAPHGVVFLHVTYVPAQEKNFTTAPAICHDGKAHFPREG | |
| | VFVSNGTHWFVTQRNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQ | |
| 37 | QCVNLTTRTQLPPAYTNSFTRGVYYPDKVFRSSVLASTQDLFLPFFSNVTWFHAIHVSGT | B.1.351 |
| | NGTKRFDNPVLPFNDGVYFASTEKSNIIRGWIFGTTLDSKTQSLLIVNNATNVVIKVCEE | South |
| | QFCNDPFLGVYYHKNNKSWMESEFRVYSSANNCTFEYVSQPFLMDLEGKQGNFKNLREFV | African |
| | FKNIDGYFKIYSKHTPINLVRDLPQGFSALEPLVDLPIGINITRFQTLLALHRSYLTPGD | variant |
| | SSSGWTAGAAAYYVGYLQPRTFLLKYNENGTITDAVDCALDPLSETKCTLKSFTVEKGIY | SARS-CoV-2 |
| | QTSNFRVQPTESIVRFPNITNLCPFGEVFNATRFASVYAWNRKRISNCVADYSVLYNSAS | spike |
| | FSTFKCYGVSPTKLNDLCFTNVYADSFVIRGDEVRQIAPGQTGNIADYNYKLPDDFTGCV | protein |
| | IAWNSNNLDSKVGGNYNYLYRLFRKSNLKPFERDISTEIYQAGSIPCNGVKGFNCYFPLQ | ectodomain |
| | SYGFQPTYGVGYQPYRVVVLSFELLHAPATVCGPKKSTNLVKNKCVNFNFNGLTGTGVLT | without |
| | ESNKKFLPFQQFGRDIADTTDAVRDPQTLEILDITPCSFGGVSVITPGTNTSNQVAVLYQ | signal |
| | GVNCTEVPVAIHADQLTPTWRVYSTGSNVFQTRAGCLIGAEHVNNSYECDIPIGAGICAS | peptide, |
| | YQTQTNSPRRAASVASQSIIAYTMSLGAENSVAYSNNSIAIPTNFTISVTTEILPVSMTK | S1/S2 furin |
| | TSVDCTMYICGDSTECSNLLLQYGSFCTQLNRALTGIAVEQDKNTQEVFAQVKQIYKTPP | cleavage |
| | IKDFGGFNFSQILPDPSKPSKRSFIEDLLFNKVTLADAGFIKQYGDCLGDISARDLICAQ | site 1 |
| | KFNGLTVLPPLLTDEMIAQYTSALLAGTITSGWTFGAGAALQIPFAMQMAYRFNGIGVTQ | mutant |
| | NVLYENQKLIANQFNSAIGKIQDSLSSTASALGKLQDVVNQNAQALNTLVKQLSSNFGAI | (685R→685A) |
| | SSVLNDILSRLDKVEAEVQIDRLITGRLQSLQTYVTQQLIRAAEIRASANLAATKMSECV | |
| | LGQSKRVDFCGKGYHLMSFPQSAPHGVVFLHVTYVPAQEKNFITAPAICHDGKAHFPREG | |
| | VFVSNGTHWFVTQRNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQ | |
| 38 | QCVNLTTRTQLPPAYTNSFTRGVYYPDKVFRSSVLHSTQDLFLPFFSNVTWFHAIHVSGT | B.1.351 |
| | NGTKRFDNPVLPFNDGVYFASTEKSNIIRGWIFGTTLDSKTQSLLIVNNATNVVIKVCEF | South |
| | QFCNDPFLGVYYHKNNKSWMESEFRVYSSANNCTFEYVSQPFLMDLEGKQGNFKNLREFV | African |
| | FKNIDGYFKIYSKHTPINLVRDLPQGFSALEPLVDLPIGINITRFQTLLALHRSYLTPGD | variant |
| | SSSGWTAGAAAYYVGYLQPRTFLLKYNENGTITDAVDCALDPLSETKCTLKSFTVEKGIY | SARS-CoV-2 |
| | QTSNFRVQPTESIVRFPNITNLCPFGEVFNATRFASVYAWNRKRISNCVADYSVLYNSAS | spike |
| | FSTFKCYGVSPTKLNDLCFTNVYADSFVIRGDEVRQIAPGQTGNIADYNYKLPDDFTGCV | protein |
| | IAWNSNNLDSKVGGNYNYLYRLFRKSNLKPFERDISTEIYQAGSTPCNGVKGFNCYFPLQ | ectodomain |
| | SYGFQPTYGVGYQPYRVVVLSFELLHAPAIVCGPKKSOTLVKNKCVNFNFNGLTGTGVLT | without |
| | ESNKKFLPFQQFGRDIADTTDAVRDPQTLEILDITPCSFGGVSVITPGTNTSNQVAVLYQ | signal |
| | GVNCTEVPVAIHADQLTPTWRVYSTGSNVFQTRAGCLIGAEHVNNSYECDIPIGAGICAS | peptide, |
| | YQTQTNSPRRARSVASQSIIAYTMSLGAENSVAYSNNSIAIPTNFTISVTTEILPVSMTK | proline |
| | TSVDCTMYICGDSTECSNLLLQYGSFCIQLNRALTGIAVEQDKNTQEVFAQVKQIYKIPP | mutant |
| | IKDFGGFNFSQILPDPSKPSKRSFIEDLLFNKVTLADAGFIKQYGDCLGDIAARDLICAQ | (986K/987V→ |
| | KFNGLTVLPPLLIDEMIAQYTSALLAGIITSGWTFGAGAALQIPFAMQMAYRFNGIGVTQ | 986P/987P) |
| | NVLYENQKLIANQFNSAIGKIQDSLSSTASALGKLQDVVNQNAQALNTLVKQLSSNFGAI | |
| | SSVLNDILSRLDPPEAEVQIDRLITGRLQSLQTYVTQQLIRAAEIRASANLAATKMSECV | |
| | LGQSKRVDFCGKGYHLMSFPQSAPHGVVFLHVTYVPAQEKNFTTAPAICHDGKAHFPREG | |
| | VFVSNGTHWFVTQRNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQ | |
| 39 | QCVNLTTRTQLPPAYTNSFTRGVYYPDKVFRSSVLHSTQDLFLPFFSNVTWFHAIHVSGT | B.1.351 |
| | NGTKRFDNPVLPFNDGVYFASTEKSNTIRGWIFGTTLDSKTQSLLIVNNATNVVIKVCEF | South |
| | QFCNDPFLGVYYHKNNKSWMESEFRVYSSANNCTFEYVSQPFLMDLEGKQGNFKNLREFV | African |
| | FKNIDGYFKIYSKHTPINLVRDLPQGFSALEPLVDLPIGINTTRFQTLLALHRSYLTPGD | variant |
| | SSSGWTAGAAAYYVGYLQPRTFLLKYNENGTITDAVDCALDPLSETKCTLKSFIVEKGIY | SARS-CoV-2 |
| | QTSNFRVQPTESIVRFPNITNLCPFGEVFNATRFASVYAWNRKRISNCVADYSVLYNSAS | spike |
| | FSTFKCYGVSPTKLNDLCFTNVYADSFVIRGDEVRQIAPGQTGNIADYNYKLPDDFTGCV | protein |
| | lAWNSNNLDSKVGGNYNYLYRLFRKSNLKPFERDISTEIYQAGSTPCNGVKGFNCYFPLQ | ectodomain |
| | SYGFOPTYGVGYQPYRVVVLSFELLHAPATVCGPKKSTNLVKNKCVNFNFNGLTGTGVLT | without |
| | ESNKKFLPFQQFGRDIADTTDAVRDPQTLEILDITPCSFGGVSVITPGTNTSNQVAVLYQ | signal |
| | GVNCTEVPVAIHADQLTPTWRVYSTGSNVFQTRAGCLIGAEHVNNSYECDIPIGAGICAS | peptide, |
| | YQTQTNSPRRAASVASQSIIAYTMSLGAENSVAYSNNSIAIPINFTISVTTEILPVSMTK | S1/S2 furin |
| | TSVDCTMYICGDSTECSNLLLQYGSFCTQLNRALTGIAVEQDKNTQEVFAQVKQIYKTPP | cleavage |
| | IKDFGGFNFSQILPDPSKPSKRSFIEDLLFNKVTLADAGFIKQYGDCLGDIAARDLICAQ | site 1 and |
| | KFNGLTVLPPLLTDEMIAQYTSALLAGTITSGWTFGAGAALQIPFAMQMAYRFNGIGVTQ | proline |
| | NVLYENQKLIANQFNSAIGKIQDSLSSTASALGKLQDVVNQNAQALNTLVKQLSSNFGAI | mutant |
| | SSVLNDILSRLDPPEAEVQIDRLITGRLQSLQTYVTQQLIRAAEIRASANLAATKMSECV | (685R→685A, |
| | LGQSKRVDFCGKGYHLMSFPQSAPHGVVFLHVTYVPAQEKNFITAPAICHDGKAHFPREG | 986K/987V→ |
| | VFVSNGTHWFVTQRNFYEPQIITTDNTFVSGNCDVVIGIVNNTVYDPLQPELDSFKEELD | 986P/987P) |
| | KYFKNHTSPDVDLGDISGINASVVNIQKEIDRLNEVAKNLNESLIDLQELGKYEQ | |
| 40 | QCVNFTNRTQLPSAYTNSFTRGVYYPDKVFRSSVLHSTQDLFLPFFSNVTWFHAIHVSGT | P.1 |
| | NGTKRFDNPVLPFNDGVYFASTEKSNIIRGWIFGTTLDSKTQSLLIVNNATNVVIKVCEF | Brazilian |
| | QFCNYPFLGVYYHKNNKSWMESEFRVYSSANNCTFEYVSQPFLMDLEGKQGNFKNLSEFV | variant |
| | FKNIDGYFKIYSKHTPINLVRDLPQGFSALEPLVDLPIGINITRFQTLLALHRSYLTPGD | SARS-CoV-2 |
| | SSSGWTAGAAAYYVGYLQPRTFLLKYNENGTITDAVDCALDPLSETKCTLKSFTVEKGIY | spike |
| | QTSNFRVOPTESIVRFPNITNLCPFGEVFNATRFASVYAWNRKRISNCVADYSVLYNSAS | protein |
| | FSTFKCYGVSPTKLNDLCFTNVYADSFVIRGDEVRQIAPGQTGTIADYNYKLPDDFTGCV | ectodomain |
| | IAWNSNNLDSKVGGNYNYLYRLFRKSNLKPFERDISTEIYQAGSTPCNGVKGFNCYFPLQ | without |
| | SYGFQPTYGVGYOPYRVVVLSFELLHAPATVCGPKKSTNLVKNKCVNFNFNGLIGTGVLT | signal |
| | ESNKKFLPFQQFGRDIADTTDAVRDPQTLEILDITPCSFGGVSVITPGTNTSNQVAVLYQ | peptide |
| | GVNCTEVPVAIHADQLTPTWRVYSTGSNVFQTRAGCLIGAEYVNNSYECDIPIGAGICAS | |
| | YQTQTNSPRRARSVASQSIIAYTMSLGAENSVAYSNNSIAIPTNFTISVTTEILPVSMTK | |
| | TSVDCTMYICGDSTECSNLLLQYGSFCTQLNRALTGIAVEQDKNTQEVFAQVKQIYKTPP | |
| | IKDFGGFNFSQILPDPSKPSKRSFIEDLLFNKVTLADAGIIKQYGDCLGDIAARDLICAQ | |
| | KFNGLTVLPPLLTDEMIAQYTSALLAGTITSGWTFGAGAALQIPFAMQMAYRFNGIGVTQ | |
| | NVLYENQKLIANQFNSAIGKIQDSLSSTASALGKLQDVVNQNAQALNTLVKQLSSNFGAI | |
| | SSVLNDILSRLDKVEAEVQIDRLITGRLQSLQTYVTQQLIRAAEIRASANLAAIKMSECV | |
| | LGQSKRVDFCGKGYHLMSFPQSAPHGVVFLHVTYVPAQEKNFTTAPAICRDGKAHFPREG | |
| | VFVSNGTHWFVTQRNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQ | |
| 41 | QCVNFTNRTQLPSAYTNSFTRGVYYPDKVFRSSVLHSTQDLFLPFFSNVTWFHAIHVSGT | P.1 |
| | NGTKRFDNPVLPFNDGVYFASTEKSNIIRGWIFGTTLDSKTQSLLIVNNATNVVIKVCEE | Brazilian |
| | QFCNYPFLGVYYHKNNKSWMESEFRVYSSANNCTFEYVSQPFLMDLEGKQGNFKNLSEFV | variant |
| | FKNIDGYFKIYSKHTPINLVRDLPQGFSALEPLVDLPIGINITRFQTLLALHRSYLTPGD | SARS-CoV-2 |
| | SSSGWTAGAAAYYVGYLQPRTFLLKYNENGTITDAVDCALDPLSETKCTLKSFTVEKGIY | spike |
| | QTSNFRVQPTESIVRFPNITNLCPFGEVFNATRFASVYAWNRKRISNCVADYSVLYNSAS | protein |
| | FSTFKCYGVSPTKLNDLCFTNVYADSFVIRGDEVRQIAPGQTGTIADYNYKLPDDFTGCV | ectodomain |
| | IAWNSNNLDSKVGGNYNYLYRLFRKSNLKPFERDISTEIYQAGSTPCNGVKGFNCYFPLQ | without |
| | SYGFQPTYGVGYQPYRVVVLSFELLHAPATVCGPKKSTNLVKNKCVNFNFNGLTGTGVLT | signal |
| | ESNKKFLPFQQFGRDIADTTDATODPQTLEILDITPCSFGGVSVITPGTNTSNQVAVLYQ | peptide, |
| | GVNCTEVPVAIHADQLTPTWRVYSTGSNVFQTRAGCLIGAEYVNNSYECDIPIGAGICAS | S1/S2 furin |
| | YQTQTNSPRRAASVASOSIIAYTMSLGAENSVAYSNNSIAIPTNFTISVTTEILPVSMTK | cleavage |
| | TSVDCTMYICGDSTECSNLLLQYGSFCTQLNRALTGIAVEQDKNTQEVFAQVKQIYKTPP | site 1 |
| | IKDFGGFNFSQILPDPSKPSKRSFIEDLLFNKVTLADAGFIKQYGDCLGDIAARDLICAQ | mu tant |
| | KFNGLTVLPPLLTDEMIAQYTSALLAGTITSGWTFGAGAALQIPFAMQMAYRFNGIGVTQ | (685R→685A) |
| | NVLYENQKLIANQFNSAIGKIQDSLSSTASALGKLQDVVNQNAQALNTLVKQLSSNFGAI | |
| | SSVLNDILSRLDKVEAEVQIDRLITGRLQSLQTYVIQQLIRAAEIRASANLAAIKMSECV | |
| | LGQSKRVDFCGKGYHLMSFPQSAPHGVVFLHVTYVPAQEKNFTTAPAICHDGKAHFPREG | |
| | VFVSNGTHWFVTQRNFYEPQIITTDNTFVSGNCDVVIGIVNNTVYDPLQPELDSFKEELD | |
| | KYFKNHTSPDVDLGDISGINASWNIQKEIDRLNEVAKNLNESLIDLQELGKYEQ | |
| 42 | QCVNFTNRTQLPSAYTNSFTRGVYYPDKVFRSSVLHSTQDLFLPFFSNVTWFUAIHVSGT | P.1 |
| | NGTKRFDNPVLPFNDGVYFASTEKSNIIRGWIFGTTLDSKTQSLLIVNNATNVVIKVCEF | Brazilian |
| | QFCNYPFLGVYYHKNNKSWMESEFRVYSSANNCTFEYVSQPFLMDLEGKQGNFKNLSEFV | variant |
| | FKNIDGYFKIYSKHTPINLVRDLPQGFSALEPLVDLPIGINITRFQTLLALHRSYLTPGD | SARS-CoV-2 |
| | SSSGWTAGAAAYYVGYLQPRTFLLKYNENGTITDAVDCALDPLSETKCTLKSFTVEKGIY | spike |
| | QTSNFRVOPTESIVRFPNITNLCPFGEVFNATRFASVYAWNRKRISNCVADYSVLYNSAS | protein |
| | FSTFKCYGVSPTKLNDLCFTNVYADSFVIRGDEVRQIAPGQTGTIADYNYKLPDDFTGCV | ectodomain |
| | IAWNSNNLDSKVGGNYNYLYRLFRKSNLKPFERDISTEIYQAGSTPCNGVKGFNCYFPLQ | without |
| | SYGFQPTYGVGYQPYRVVVLSFELLHAPATVCGPKKSTNLVKNKCVNFNFNGLTGTGVLT | signal |
| | ESNKKFLPFQQFGRDIADTTDAVRDPQTLEILDITPCSFGGVSVITPGTNTSNQVAVLYQ | peptide, |
| | GVNCTEVPVAIHADQLTPTWRVYSTGSNVFQTRAGCLIGAEYVNNSYECDIPIGAGICAS | proline |
| | YQTQTNSPRRARSVASQSIIAYTMSLGAENSVAYSNNSIAIPTNFTISVTTEILPVSMTK | mutant |
| | TSVDCTMYICGDSTECSNLLLQYGSFCTQLNRALTGIAVEQDKNTQEVFAQVKQIYKTPP | (986K/987V→ |
| | IKDFGGFNFSQILPDPSKPSKRSFIEDLLFNKVTLADAGFIKQYGDCLGDIAARDLICAQ | 986P/987P) |
| | KFNGLTVLPPLLTDEMIAQYTSALLAGTITSGWTFGAGAALQIPFAMQMAYRFNGIGVTQ | |
| | NVLYENQKLIANQFNSAIGKIQDSLSSTASALGKLQDVVNQNAQALNTLVKQLSSNFGAI | |
| | SSVLNDILSRLDPPEAEVQIDRLITGRLQSLQTYVTQQLIRAAEIRASANLAAIKMSECV | |
| | LGQSKRVDFCGKGYHLMSFPQSAPHGVVFLHVTYVPAQEKNFTTAPAICHDGKAHFPREG | |
| | VFVSNGTHWFVTQRNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQ | |
| 43 | QCVNFTNRTQLPSAYTNSFTRGVYYPDKVFRSSVLHSTQDLFLPFFSNVTWFHAIHVSGT | P.1 |
| | NGTKRFDNPVLPFNDGVYFASTEKSNIIRGWIFGTTLDSKTQSLLIVNNATNVVIKVCEF | Brazilian |
| | QFCNYPFLGVYYHKNNKSWMESEFRVYSSANNCTFEYVSQPFLMDLEGKQGNFKNLSEFV | variant |
| | FKNIDGYFKIYSKHTPINLVRDLPQGFSALEPLVDLPIGINITRFQTLLALHRSYLTPGD | SARS-CoV-2 |
| | SSSGWTAGAAAYYVGYLQPRTFLLKYNENGTITDAVDCALDPLSETKCTLKSFTVEKGIY | spike |
| | QTSNFRVQPTESIVRFPNITNLCPFGEVFNATRFASVYAWNRKRISNCVADYSVLYNSAS | protein |
| | FSTFKCYGVSPTKLNDLCFTNVYADSFVIRGDEVRQIAPGQTGTIADYNYKLPDDFTGCV | ectodomain |
| | IAWNSNNLDSKVGGNYNYLYRLFRKSNLKPFERDISTEIYQAGSTPCNGVKGFNCYFPLQ | without |
| | SYGFQPTYGVGYOPYRVVVLSFELLHAPATVCGPKKSTNLVKNKCVNFNFNGLIGTGVLT | signal |
| | ESNKKFLPFQQFGRDIADTTDAVRDPQTLEILDITPCSFGGVSVITPGTNTSNQVAVLYQ | peptide, |
| | GVNCTEVPVAIHADQLTPTWRVYSTGSNVFQTRAGCLIGAEYVNNSYECDIPIGAGICAS | S1/S2 furin |
| | YQTQTNSPRRAASVASQSIIAYTMSLGAENSVAYSNNSIAIPTNFTISVTTEILPVSMTK | cleavage |
| | TSVDCTMYICGDSTECSNLLLQYGSFCTQLNRALTGIAVEQDKNTQEVFAQVKQIYKTPP | site 1 and |
| | IKDFGGFNFSQILPDPSKPSKRSFIEDLLFNKVTLADAGFTKQYGDCLGDIAARDLICAQ | proline |
| | KFNGLTVLPPLLIDEMIAQYTSALLAGTITSGWTFGAGAALQIPFAMQMAYRFNGIGVTQ | mutant. |
| | NVLYENQKLIANQFNSAIGKIQDSLSSTASALGKLQDVVNQNAQALNTLVKQLSSNFGAI | (685R→685A, |
| | SSVLNDILSRLDPPEAEVQIDRLITGRLQSLQTYVTQQLIRAAEIRASANLAAIKMSECV | 986K/987V→ |
| | LGQSKRVDFCGKGYHLMSFPQSAPHGVVFLHVTYVPAQEKNFTTAPAICHDGKAHFPREG | 986P/987P) |
| | VFVSNGTHWFVTQRNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQ | |
| 44 | QCVNLTTRTQLPPAYTNSFTRGVYYPDKVFRSSVLHSTQDLFLPFFSNVTWFHAISGTNG | B.1.1.7 UK |
| | TKRFDNPVLPFNDGVYFASTEKSNIIRGWIFGTTLDSKTQSLLIVNNATNVVIKVCEFQF | variant |
| | CNDPFLGVYYHKNNKSWMESEFRVYSSANNCTFEYVSQPFLMDLEGKQGNFKNLREFVFK | SARS-CoV-2 |
| | NIDGYFKIYSKHTPINLVRDLPQGFSALEPLVDLPIGINITRFQTLLALHRSYLTPGDSS | spike |
| | SGWTAGAAAYYVGYLQPRTFLLKYNENGTITDAVDCALDPLSETKCTLKSFTVEKGIYQT | protein |
| | SNFRVQPTESIVRFPNITNLCPFGEVFNATRFASVYAWNRKRISNCVADYSVLYNSASFS | ectodomain |
| | TFKCYGVSPTKLNDLCFTNVYADSFVIRGDEVRQIAPGQTGKIADYNYKLPDDFTGCVIA | without |
| | WNSNNLDSKVGGNYNYLYRLFRKSNLKPFERDISTEIYQAGSTPCNGVEGFNCYFPLQSY | signal |
| | GFQPTYGVGYQPYRVVVLSFELLHAPATVCGPKKSTNLVKNKCVNFNFNGLTGTGVLTES | peptide |
| | NKKFLPFQQFGRDIADTTDAVRDPQTLEILDITPCSFGGVSVITPGTNTSNQVAVLYQGV | |
| | NCTEVPVAIHADQLTPTWRVYSTGSNVFQTRAGCLIGAEHVNNSYECDIPIGAGICASYQ | |
| | TQTNSHRRARSVASQSIIAYTMSLGAENSVAYSNNSIAIPTNFTISVTTEILPVSMTKTS | |
| | VDCTMYICGDSTECSNLLLQYGSFCTQLNRALTGIAVEQDKNTQEVFAQVKQIYKTPPIK | |
| | DFGGFNFSQILPDPSKPSKRSFIEDLLFNKVTLADAGFIKQYGDCLGDIAARDLICAQKE | |
| | NGLTVLPPLLIDEMIAQYISALLAGTITSGWTFGAGAALQIPFAMQMAYRFNGIGVTQNV | |
| | LYENQKLIANQFNSAIGKIQDSLSSTASALGKLQDVVNQNAQALNTLVKQLSSNFGAISS | |
| | VLNDILSRLDKVEAEVQIDRLITGRLQSLQTYVTQQLIRAAEIRASANLAATKMSECVLG | |
| | QSKRVDFCGKGYHLMSFPQSAPHGVVFLHVTYVPAQEKNFTTAPAICHDGKAHFPREGVF | |
| | VSNGTHWFVTQRNFYEPQIITTDNTFVSGNCDVVIGIVNNTVYDPLQPELDSFKEELDKY | |
| | FKNHTSPDVDLGDISGINASVVNIQKEIDRLNEVAKNLNESLIDLQELGKYEQ | |
| 45 | QCVNLTTRTQLPPAYTNSFTRGVYYPDKVFRSSVLHSTQDLFLPFFSNVTWFHAISGTNG | B.1.1.7 UK |
| | TKRFDNPVLPFNDGVYFASTEKSNIIRGWIFGTTLDSKTQSLLIVNNATNVVIKVCEFQF | variant |
| | CNDPFLGVYYHKNNKSWMESEFRVYSSANNCTFEYVSQPFLMDLEGKQGNFKNLREFVFK | SARS-CoV-2 |
| | NIDGYFK1YSKHTPINLVRBLPQGFSALEPLVDLPIG1NITRFQTLLALHRSYLTPGDSS | spike |
| | SGWTAGAAAYYVGYLQPRTFLLKYNENGTITDAVDCALDPLSETKCTLKSFTVEKGIYQT | protein |
| | SNFRVQPTESIVRFPNITNLCPFGEVFNATRFASVYAWNRKRISNCVADYSVLYNSASFS | ectodomain |
| | TFKCYGVSPTKLNDLCFTNVYADSFVIRGDEVRQIAPGQTGKIADYNYKLPDDFTGCVIA | without |
| | WNSNNLDSKVGGNYNYLYRLFRKSNLKPFERDISTEIYQAGSTPCNGVEGFNCYFPLQSY | signal |
| | GFQPTYGVGYQPYRVVVLSFELLHAPATVCGPKKSTNLVKNKCVNFNFNGLTGTGVLTES | peptide, |
| | NKKFLPFQQFGRDIADTTDAVRDPQTLEILDITPCSFGGVSVITPGTNTSNQVAVLYOGV | S1/S2 furin |
| | NCTEVPVAIHADQLTPTWRVYSTGSNVFQTRAGCLIGAEHVNNSYECDIPIGAGICASYQ | cleavage |
| | TQTNSHRRAASVASOSIIAYTMSLGAENSVAYSNNSIAIPTNFTISVTTEILPVSMTKTS | site 1 |
| | VDCTMYICGDSTECSNLLLQYGSFCTQLNRALTGIAVEQDKNTQEVFAQVKQIYKTPPIK | mutant |
| | DFGGFNFSQILPDPSKPSKRSFIEDLLFNKVTLADAGFIKQYGDCLGDIAARDLICAQKF | (685R→685A) |
| | NGLTVLPPLLTDEMIAQYTSALLAGTITSGWTFGAGAALQIPFAMQMAYRFNGIGVTQNV | |
| | LYENQKLIANQFNSAIGKIQDSLSSTASALGKLQDVVNQNAQALNTLVKQLSSNFGAISS | |
| | VLNDILSRLDKVEAEVQIDRLITGRLQSLQTYVTQQLIRAAEIRASANLAATKMSECVLG | |
| | QSKRVDFCGKGYHLMSFPQSAPHGVVFLHVTYVPAQEKNFTTAPAICHDGKAHFPREGVF | |
| | VSNGTHWFVTQRNFYEPQTITTDNTFVSGNCDVVTGIVNNTVYDPLQPELDSFKEELDKY | |
| | FKNHTSPDVDLGDISGINASVVNIOKEIDRLNEVAKNLNESLIDLQELGKYEQ | |
| 46 | QCVNLTTRTQLPPAYTNSFTRGVYYPDKVFRSSVLHSTQDLFLPFFSNVTWFHAISGTNG | B.1.1.7 UK |
| | TKRFDNPVLPFNDGVYFASTEKSNIIRGWIFGTTLDSKTQSLLIVNNATNVVIKVCEFQF | variant |
| | CNDPFLGVYYHKNNKSWMESEFRVYSSANNCTFEYVSQPFLMDLEGKQGNFKNLREFVFK | SARS-CoV-2 |
| | NIDGYFKIYSKHTPINLVRDLPQGFSALEPLVDLPIGINITRFQILLALHRSYLTPGDSS | spike |
| | SGWTAGAAAYYVGYLQPRTFLLKYNENGTITDAVDCALDPLSETKCTLKSFTVEKGIYQI | protein |
| | SNFRVQPTESIVRFPNITNLCPFGEVFNATRFASVYAWNRKRISNCVADYSVLYNSASFS | ectodomain |
| | TFKCYGVSPTKLNDLCFTNVYADSFVIRGDEVRQIAPGQTGKIADYNYKLPDDFTGCVIA | without |
| | WNSNNLDSKVGGNYNYLYRLFRKSNLKPFERDISTEIYQAGSTPCNGVEGFNCYFPLQSY | signal |
| | GFQPTYGVGYQPYRVVVLSFELLHAPATVCGPKKSTNLVKNKCVNFNFNGLTGTGVLTES | peptide, |
| | NKKFLPFQQFGRDIADTTDAVRDPQTLEILDITPCSFGGVSVITPGTNTSNQVAVLYOGV | proline |
| | NCTEVPVAIHADQLTPTWRVYSTGSNVFQTRAGCLIGAEHVNNSYECDIPIGAGICASYQ | mutant |
| | TQTNSHRRARSVASQSIIAYTMSLGAENSVAYSNNSIAIPTNFTISVTTEILPVSMTKTS | (986K/987V→ |
| | VDCTMYICGDSTECSNLLLQYGSFCTQLNRALTGIAVEQDKNTQEVFAQVKQIYKTPPIK | 986P/987P) |
| | DFGGFNFSQILPDPSKPSKRSFIEDLLFNKVTLADAGFIKQYGDCLGDIAARDLICAQKF | |
| | NGLTVLPPLLTDEMIAQYTSALLAGTITSGWTFGAGAALQIPFAMOMAYRFNGIGVTQNV | |
| | LYENQKLIANQFNSAIGKIQDSLSSTASALGKLQDVVNQNAQALNTLVKQLSSNFGAISS | |
| | VLNDILSRLDPPEAEVQIDRLITGRLQSLQTYVTQQLIRAAEIRASANLAATKMSECVLG | |
| | QSKRVDFCGKGYHLMSFPQSAPHGVVFLHVTYVPAQEKNFTTAPAICHDGKAHFPREGVF | |
| | VSNGTHWFVTQRNFYEPQIITTDNTFVSGNCDVVIGIVNNTVYDPLQPELDSFKEELDKY | |
| | FKNHTSPDVDLGDISGINASVVNIQKEIDRLNEVAKNLNESLIDLQELGKYEQ | |
| 47 | QCVNLTTRTQLPPAYTNSFTRGVYYPDKVFRSSVLHSTQDLFLPFFSNVTWFHAISGTNG | B.1.1.7 UK |
| | TKRFDNPVLPFNDGVYFASTEKSNIIRGWIFGTTLDSKTQSLLIVNNATNVVIKVCEFQF | variant |
| | CNDPFLGVYYHKNNKSWMESEFRVYSSANNCTFEYVSQPFLMDLEGKQGNFKNLREFVFK | SARS-CoV-2 |
| | NIDGYFKIYSKHIPINLVRDLPQGFSALEPLVDLPIGINITRFQTLLALHRSYLTPGDSS | spike |
| | SGWTAGAAAYYVGYLQPRTFLLKYNENGTITDAVDCALDPLSETKCTLKSFTVEKGIYQT | protein |
| | SNFRVQPTESIVRFPNITNLCPFGEVFNATRFASVYAWNRKRISNCVADYSVLYNSASFS | ectodomain |
| | TFKCYGVSPTKLNDLCFTNVYADSFVIRGDEVRQIAPGQTGKIADYNYKLPDDFTGCVIA | without |
| | WNSNNLDSKVGGNYNYLYRLFRKSNLKPFERDISTEIYQAGSTPCNGVEGFNCYFPLQSY | signal |
| | GFQPTYGVGYQPYRVVVLSFELLHAPATVCGPKKSTNLVKNKCVNFNFNGLTGTGVLTES | peptide, |
| | NKKFLPFQQFGRDIADTTDAVRDPQTLEILDITPCSFGGVSVITPGTNTSNQVAVLYQGV | S1/S2 furin |
| | NCTEVPVAIHADQLTPTWRVYSTGSNVFQTRAGCLIGAEHVNNSYECDIPIGAGICASYQ | cleavage |
| | IQTNSHRRAASVASQSIIAYTMSLGAENSVAYSNNSIAIPTNFTISVTTEILPVSMTKTS | site 1 and |
| | VDCTMYICGDSTECSNLLLQYGSFCTQLNRALTGIAVEQDKNTQEVFAQVKQIYKTPPIK | proline |
| | DFGGFNFSQILPDPSKPSKRSFIEDLLFNKVTLADAGFIKQYGDCLGDIAARDLICAQKF | mutant |
| | NGLTVLPPLLIDEMIAQYTSALLAGIITSGWTFGAGAALQIPFAMQMAYRFNGIGVTQNV | (685R→685A, |
| | LYENQKLIANQFNSAIGKIQDSLSSTASALGKLQDVVNQNAQALNTLVKQLSSNFGAISS | 986K/987V→ |
| | VLNDILSRLDPPEAEVQIDRLITGRLQSLQTYVTQQLIRAAEIRASANLAATKMSECVLG | 986P/987P) |
| | QSKRVDFCGKGYHLMSFPQSAPHGVVFLHVTYVPAQEKNFTTAPAICHDGKAHFPREGVF | |
| | VSNGTHWFVTQRNFYEPQIITTDNTFVSGNCDVVIGIVNNTVYDPLQPELDSFKEELDKY | |
| | FKNHTSPDVDLGDISGINASVVNIQKEIDRLNEVAKNLNESLIDLQELGKYEQ | |
| 48 | QCVNLTTRTQLPPAYTNSFTRGVYYPDKVFRSSVLHSTQDLFLPFFSNVTWFHAIHVSGT | D614G |
| | NGTKRFDNPVLPFNDGVYFASTEKSNIIRGWIFGTTLDSKTQSLLIVNNATNVVIKVCEF | variant |
| | QFCNDPFLGVYYHKNNKSWMESEFRVYSSANNCTFEYVSQPFLMDLEGKQGNFKNLREFV | SARS-CoV-2 |
| | FKNIDGYFKIYSKHTPINLVRDLPQGFSALEPLVDLPIGINITRFQTLLALHRSYLTPGD | spike |
| | SSSGWTAGAAAYYVGYLQPRTFLLKYNENGTITDAVDCALDPLSETKCTLKSFTVEKGIY | protein |
| | QTSNFRVQPTESIVRFPNITNLCPFGEVFNATRFASVYAWNRKRISNCVADYSVLYNSAS | ectodomain |
| | FSTFKCYGVSPTKLNDLCFTNVYADSFVIRGDEVRQIAPGQTGKIADYNYKLPDDFTGCV | without |
| | IAWNSNNLDSKVGGNYNYLYRLFRKSNLKPFERDISTEIYQAGSTPCNGVEGFNCYFPLQ | signal |
| | SYGFQPTNGVGYQPYRVVVLSFELLHAPAIVCGPKKSTNLVKNKCVNFNFNGLTGTGVLT | peptide |
| | ESNKKFLPFQQFGRDIADTTDAVRDPQTLEILDITPCSFGGVSV1TPGTNTSNQVAVLYQ | |
| | GVNCTEVPVAIHADQLTPTWRVYSTGSNVFQTRAGCLIGAEHVNNSYECDIPIGAGICAS | |
| | YQTQTNSPRRARSVASQSIIAYTMSLGAENSVAYSNNSIAIPTNFTISVTTEILPVSMTK | |
| | TSVDCTMYICGDSTECSNLLLQYGSFCTQLNRALTGIAVEQDKNTQEVFAQVKQIYKIPP | |
| | IKDFGGFNFSQILPDPSKPSKRSFIEDLLFNKVTLADAGFIKQYGDCLGDIAARDLICAQ | |
| | KFNGLTVLPPLLIDEMIAQYTSALLAGIITSGWTFGAGAALQIPFAMQMAYRFNGIGVTQ | |
| | NVLYENQKLIANQFNSAIGKIQDSLSSTASALGKLQDVVNQNAQALNTLVKQLSSNFGAI | |
| | SSVLNDILSRLDKVEAEVQIDRLITGRLQSLQTYVTQQLIRAAEIRASANLAATKMSECV | |
| | LGQSKRVDFCGKGYHLMSFPQSAPHGVVFLHVTYVPAQEKNFTTAPAICHDGKAHFPREG | |
| | VFVSNGTHWFVTORNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQ | |
| 49 | QCVNLTTRTQLPPAYTNSFTRGVYYPDKVFRSSVLASTQDLFLPFFSNVTWFHAIHVSGT | D614G |
| | NGTKRFDNPVLPFNDGVYFASTEKSNIIRGWIFGTTLDSKTQSLLIVNNATNVVIKVCEF | variant |
| | QFCNDPFLGVYYHKNNKSWMESEFRVYSSANNCTFEYVSQPFLMDLEGKQGNFKNLREFV | SARS-CoV-2 |
| | FKNIDGYFKIYSKHTPINLVRDLPQGFSALEPLVDLPIGINITRFQTLLALHRSYLTPGD | spike |
| | SSSGWTAGAAAYYVGYLQPRTFLLKYNENGTITDAVDCALDPLSETKCTLKSFTVEKGIY | protein |
| | QTSNFRVQPTESIVRFPNITNLCPFGEVFNATRFASVYAWNRKRISNCVADYSVLYNSAS | ectodomain |
| | FSTFKCYGVSPTKLNDLCFTNVYADSFVIRGDEVRQIAPGQTGKIADYNYKLPDDFTGCV | without |
| | IAWNSNNLDSKVGGNYNYLYRLFRKSNLKPFERDISTEIYQAGSIPCNGVEGFNCYFPLQ | signal |
| | SYGFQPTNGVGYQPYRVVVLSFELLHAPATVCGPKKSTNLVKNKCVNFNFNGLTGTGVLI | peptide, |
| | ESNKKFLPFQQFGRDIADTTDAVRDPQTLEILDITPCSFGGVSVITPGTNTSNQVAVLYQ | S1/S2 furin |
| | GVNCTEVPVAIHADQLTPTWRVYSTGSNVFQTRAGCLIGAEHVNNSYECDIPIGAGICAS | cleavage |
| | YQTQINSPRRAASVASQSIIAYTMSLGAENSVAYSNNSIAIPTNFTISVTTEILPVSMTK | site 1 |
| | TSVDCTMYICGDSTECSNLLLQYGSFCTQLNRALTGIAVEQDKNTQEVFAQVKQIYKTPP | mutant |
| | IKDFGGFNFSQILPDPSKPSKRSFIEDLLFNKVTLADAGFIKQYGDCLGDIAARDLICAQ | (685R→685A) |
| | KFNGLTVLPPLLTDEMIAQYTSALLAGTITSGWTFGAGAALQIPFAMQMAYRFNGIGVTQ | |
| | NVLYENQKLIANQFNSAIGKIQDSLSSTASALGKLQDVVNQNAQALNTLVKQLSSNFGAI | |
| | SSVLNDILSRLDKVEAEVQIDRLITGRLQSLQTYVTQQLIRAAEIRASANLAATKMSECV | |
| | LGQSKRVDFCGKGYHLMSFPQSAPHGVVFLHVTYVPAQEKNFTTAPAICHDGKAHFPREG | |
| | VFVSNGTHWFVTQRNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQ | |
| 50 | QCVNLTTRTQLPPAYTNSFTRGVYYPDKVFRSSVLHSTQDLFLPFFSNVTWFHAIHVSGT | D614G |
| | NGTKRFDNPVLPFNDGVYFASTEKSNIIRGWIFGTTLDSKTQSLLIVNNATNVVIKVCEF | variant |
| | QFCNDPFLGVYYHKNNKSWMESEFRVYSSANNCTFEYVSQPFLMDLEGKQGNFKNLREFV | SARS-CoV-2 |
| | FKNIDGYFKIYSKHTPINLVRDLPQGFSALEPLVDLPIGINITRFQTLLALHRSYLTPGD | spike |
| | SSSGWTAGAAAYYVGYLQPRTFLLKYNENGTITDAVDCALDPLSETKCTLKSFTVEKGIY | protein |
| | QTSNFRVQPTESIVRFPNITNLCPFGEVFNATRFASVYAWNRKRISNCVADYSVLYNSAS | ectodomain |
| | FSTFKCYGVSPTKLNDLCFTNVYADSFVIRGDEVRQIAPGQTGKIADYNYKLPDDFTGCV | without |
| | IAWNSNNLDSKVGGNYNYLYRLFRKSNLKPFERDISTEIYQAGSTPCNGVEGFNCYFPLQ | signal |
| | SYGFQPTNGVGYQPYRVVVLSFELLHAPATVCGPKKSTNLVKNKCVNFNFNGLTGTGVLT | peptide, |
| | ESNKKFLPFQQFGRDIADTTDAVRDPQTLEILDITPCSFGGVSVITPGTNTSNQVAVLYQ | proline |
| | GVNCTEVPVAIHADQLTPTWRVYSTGSNVFQTRAGCLIGAEHVNNSYECDIPIGAGICAS | mutant |
| | YQTQTNSPRRARSVASQSIIAYTMSLGAENSVAYSNNSIAIPTNFTISVTTEILPVSMTK | (986K/987V→ |
| | TSVDCTMYICGDSTECSNLLLQYGSFCTQLNRALTGIAVEQDKNTQEVFAQVKQIYKTPP | 986P/987P) |
| | IKDFGGFNFSQILPDPSKPSKRSFIEDLLFNKVTLADAGFIKQYGDCLGDIAARDLICAQ | |
| | KFNGLTVLPPLLTDEMIAQYTSALLAGTITSGWTFGAGAALQIPFAMQMAYRFNGIGVTQ | |
| | NVLYENQKLIANQFNSAIGKIQDSLSSTASALGKLQDVVNQNAQALNTLVKQLSSNFGAI | |
| | SSVLNDILSRLDPPEAEVQIDRLITGRLQSLQTYVTQQLIRAAEIRASANLAATKMSECV | |
| | LGQSKRVDFCGKGYHLMSFPQSAPHGVVFLHVTYVPAQEKNFTTAPAICHDGKAHFPREG | |
| | VFVSNGTHWFVTQRNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQ | |
| 51 | QCVNLTTRTQLPPAYTNSFTRGVYYPDKVFRSSVLHSTQDLFLPFFSNVTWFHAIHVSGT | D614G |
| | NGTKRFDNPVLPFNDGVYFASTEKSNIIRGWIFGTTLDSKTQSLLIVNNATNVVIKVCEF | variant |
| | QFCNDPFLGVYYHKNNKSWMESEFRVYSSANNCTFEYVSQPFLMDLEGKQGNFKNLREFV | SARS-CoV-2 |
| | FKNIDGYFKIYSKHTPINLVRDLPQGFSALEPLVDLPIGINITRFQTLLALHRSYLTPGD | spike |
| | SSSGWTAGAAAYYVGYLQPRTFLLKYNENGTITDAVDCALDPLSETKCTLKSFTVEKGIY | protein |
| | QTSNFRVQPTESIVRFPNITNLCPFGEVFNATRFASVYAWNRKRISNCVADYSVLYNSAS | ectodomain |
| | FSTFKCYGVSPTKLNDLCFTNVYADSFVIRGDEVRQIAPGQTGKIADYNYKLPDDFTGCV | without |
| | IAWNSNNLDSKVGGNYNYLYRLFRKSNLKPFERDISTEIYQAGSTPCNGVEGFNCYFPLQ | signal |
| | SYGFQPTNGVGYQPYRVVVLSFELLHAPATVCGPKKSTNLVKNKCVNFNFNGLTGTGVLI | peptide, |
| | ESNKKFLPFQQFGRDIADTTDAVRDPQTLEILDITPCSFGGVSVITPGTNTSNQVAVLYQ | S1/S2 furin |
| | GVNCTEVPVAIHADQLTPTWRVYSTGSNVFQTRAGCLIGAEHVNNSYECDIPIGAGICAS | cleavage |
| | YQTQTNSPRRAASVASQSIIAYTMSLGAENSVAYSNNSIAIPTNFTISVTTEILPVSMTK | site 1 and |
| | TSVDCTMYICGDSTECSNLLLQYGSFCTQLNRALTGIAVEQDKNTQEVFAQVKQIYKTPP | proline |
| | IKDFGGFNFSQILPDPSKPSKRSFIEDLLFNKVTLADAGFIKQYGDCLGDIAARDLICAQ | mutant |
| | KFNGLTVLPPLLTDEMIAQYTSALLAGTITSGWTFGAGAALQIPFAMQMAYRFNGIGVTQ | (685R→685A, |
| | NVLYENQKLIANQFNSAIGKIQDSLSSTASALGKLQDVVNQNAQALNTLVKQLSSNFGAI | 986K/987V→ |
| | SSVLNDILSRLDPPEAEVQIDRLITGRLQSLQTYVTQQLIRAAEIRASANLAATKMSECV | 986P/987P) |
| | LGQSKRVDFCGKGYHLMSFPQSAPHGVVFLHVTYVPAQEKNFTTAPAICHDGKAHFPREG | |
| | VFVSNGTHWFVTORNFYEPQIITTDNTFVSGNCDVVIGIVNNTVYDPLQPELDSFKEELD | |
| | KYFKNHTSPDVDLGDISGINASVVNIQKEIDRLNEVAKNLNESLIDLQELGKYEQ | |
| 52 | SDLDRCTTFDDVQAPNYTQHTSSMRGVYYPDEIFRSDTLYLTQDLFLPFYSNVTGFHTIN | SARS-CoV-1 |
| | HTFDNPVIPFKDGIYFAATEKSNVVRGWVFGSTMNNKSQSV11INNSTNVVIRACNFELC | spike |
| | DNPFFAVSKPMGTQTHTMIFDNAFNCTFEYISDAFSLDVSEKSGNFKRLREFVFKNKDGF | protein |
| | LYVYKGYQPIDVVRDLPSGFNTLKPIFKLPLGINITNFRAILTAFLPAQDTWGISAAAYF | ectodomain |
| | VGYLKPTTFMLKYDENGTITDAVDCSQNPLAELKCSVKSFEIDKGIYQTSNFRVVPSRDV | witnout |
| | VRFPNITNLCPFGEVFNATKFPSVYAWERKRISNCVADYSVLYNSTFFSTFKCYGVSATK | signal |
| | LNDLCFSNVYADSFVVKGDDVRQIAPGQTGVIADYNYKLPDDFMGCVLAWNTRNIDATST | peptide |
| | GNYNYKYRYLRHGKLRPFERDISNVPFSPDGKPCTPPALNCYWPLNDYGFYTTTGIGYQP | |
| | YRVVVLSFELLNAPATVCGPKLSTDLIKNQCVNFNFNGLTGTGVLTPSSKRFQPFQQFGR | |
| | DVSDFTDSVRDPKTSE1LDISPCSFGGVSVITPGTNASSEVAVLYQDVNCTDVSTAIHAD | |
| | QLTPAWRIYSTGNNVFQTQAGCLlGAEHVDTSYECDIPlGAGlCASYHTVSLLRSTSQKS | |
| | IVAYTMSLGADSSIAYSNNTIAIPTNFSISITTEVMPVSMAKTSVDCNMYICGDSTECAN | |
| | LLLQYGSFCTQLNRALSGIAAEQDRNTREVFAQVKQMYKTPTLKDFGGFNFSQILPDPLK | |
| | PTKRSFIEDLLFNKVTLADAGFMKQYGECLGDINARDLICAQKFNGLIVLPPLLTDDMIA | |
| | AYTAALVSGTATAGWTFGAGAALQIPFAMQMAYRFNGIGVTQNVLYENQKQIANQFNKAI | |
| | SQIQESLTTTSTALGKLQDVVNQNAQALNTLVKQLSSNFGAISSVLNDILSRLDKVEAEV | |
| | QIDRLITGRLQSLQTYVTQQLIRAAEIRASANLAATKMSECVLGQSKRVDFCGKGYHLMS | |
| | FPQAAPHGVVFLHVTYVPSQERNFTTAPAICHEGKAYFPREGVFVFNGTSWFITQRNFFS | |
| | PQIITTDNTFVSGNCDVVIGIINNTVYDPLQPELDSFKEELDKYFKNHTSPDVDLGDISG | |
| | INASVVNIQEEIDRLNEVAKNLNESLIDLQELGKYEQ | |
| 53 | MFIFLLFLILTSG | SARS-CoV-1 |
| | | spike |
| | | protein |
| | | signal |
| | | peptide |
| 54 | MFVFLVLLPLVSS | Prototypic |
| | | SARS-CoV-2 |
| | | spike |
| | | protein |
| | | signal |
| | | peptide |
| 55 | MFVFLVLLPLVSSQCVNLTTRTQLPPAYTNSFTRGVYYPDKVFRSSVLHSTODLFLPFFS | Prototypic |
| | NVTWFHAIHVSGTNGTKRFDNPVLPFNDGVYFASTEKSNIIRGWIFGTTLDSKTQSLLIV | SARS-CoV-2 |
| | NNATNVVIKVCEFQFCNDPFLGVYYHKNNKSWMESEFRVYSSANNCTFEYVSQPFLMDLE | full-length |
| | GKQGNFKNLREFVFKNIDGYFKIYSKHTPINLVRDLPQGFSALEPLVDLPIGINITRFQT | spike |
| | LLALHRSYLTPGDSSSGWTAGAAAYYVGYLQPRTFLLKYNENGTITDAVDCALDPLSETK | protein, |
| | CTLKSFTVEKGIYQTSNFRVQPTESIVRFPNITNLCPFGEVFNATRFASVYAWNRKRISN | 1273 aa |
| | CVADYSVLYNSASFSTFKCYGVSPTKLNDLCFINVYADSFVIRGDEVRQIAPGQTGKIAD | |
| | YNYKLPDDFTGCVIAWNSNNLDSKVGGNYNYLYRLFRKSNLKPFERDISTEIYQAGSTPC | |
| | NGVEGFNCYFPLQSYGFQPTNGVGYQPYRVVVLSFELLHAPATVCGPKKSTNLVKNKCVN | |
| | FNFNGLTGTGVLTESNKKFLPFQQFGRD1ADTTDAVRDPQTLEILDITPCSFGGVSVITP | |
| | GTNTSNQVAVLYQDVNCTEVPVAIHADQLTPTWRVYSTGSNVFQTRAGCLIGAEHVNNSY | |
| | ECDIPIGAGICASYQTQTNSPRRARSVASQSIIAYTMSLGAENSVAYSNNSIAIPTNFTI | |
| | SVTTEILPVSMTKTSVDCTMYICGDSTECSNLLLQYGSFCTQLNRALTGIAVEQDKNTQE | |
| | VFAQVKQIYKTPPIKDFGGFNFSQILPDPSKPSKRSFIEDLLFNKVTLADAGFIKQYGDC | |
| | LGDIAARDLICAQKFNGLTVLPPLLTDEMTAQYTSALLAGTITSGWTFGAGAALQIPFAM | |
| | QMAYRFNGIGVTQNVLYENQKLIANQFNSAIGKIQDSLSSTASALGKLQDVVNQNAQALN | |
| | TLVKQLSSNFGAISSVLNDILSRLDKVEAEVQIDRLITGRLQSLQTYVTQQLIRAAEIRA | |
| | SANLAATKMSECVLGQSKRVDFCGKGYHLMSFPQSAPHGVVFLHVTYVPAQEKNFTTAPA | |
| | ICHDGKAHFPREGVFVSNGTHWFVTQRNFYEPQIITIDNTFVSGNCDVVIGIVNNTVYDP | |
| | LQPELDSFKEELDKYFKNHTSPDVDLGDISGINASVVNIQKEIDRLNEVAKNLNESLIDL | |
| | QELGKYEQYIKWPWYIWLGFIAGLIAIVMVTIMLCCMTSCCSCLKGCCSCGSCCKFDEDD | |
| | SEPVLKGVKLHYT | |
| 56 | MFVFLVLLPLVSSQCVNLTTRTQLPPAYTNSFTRGVYYPDKVFRSSVLHSTQDLFLPFFS | Prototypic |
| | NVTWFHAIAVSGTNGTKRFDNPVLPFNDGVYFASTEKSNIIRGWIFGTTLDSKTQSLLIV | SARS-CoV-2 |
| | NNATNVVIKVCEFQFCNDPFLGVYYHKNNKSWMESEFRVYSSANNCTFEYVSQPFLMDLE | spike |
| | GKQGNFKNLREFVFKNIDGYFKIYSKHTPINLVRDLPQGFSALEPLVDLPIGINIIRFQI | protein |
| | LLALHRSYLTPGDSSSGWTAGAAAYYVGYLQPRTFLLKYNENGTITDAVDCALDPLSETK | ectodomain |
| | CTLKSFTVEKGIYQTSNFRVOPTESIVRFPNITNLCPFGEVFNATRFASVYAWNRKRISN | with signal |
| | CVADYSVLYNSASFSTFKCYGVSPTKLNDLCFTNVYADSFVIRGDEVRQIAPGQTGKIAD | peptide |
| | YNYKLPDDFTGCVIAWNSNNLDSKVGGNYNYLYRLFRKSNLKPFERDISTEIYQAGSTPC | |
| | NGVEGFNCYFPLQSYGFQPTNGVGYQPYRVVVLSFELLHAPATVCGPKKSTNLVKNKCVN | |
| | FNFNGLTGTGVLTESNKKFLPFQQFGRDIADTTDAVRDPQTLEILDITPCSFGGVSVITP | |
| | GTNTSNQVAVLYQDVNCTEVPVAIHADQLTPTWRVYSTGSNVFQTRAGCLIGAEHVNNSY | |
| | ECDIPIGAGICASYQTQTNSPRRARSVASQSIIAYTMSLGAENSVAYSNNSIAIPTNFTI | |
| | SVTTEILPVSMTKTSVDCTMYICGDSTECSNLLLQYGSFCTQLNRALTGIAVEQDKNTQE | |
| | VFAQVKQIYKTPPIKDFGGFNFSQILPDPSKPSKRSFLEDLLFNKVTLADAGFIKQYGDC | |
| | LGDIAARDLICAQKFNGLTVLPPLLTDEMIAQYTSALLAGTITSGWTFGAGAALQIPFAM | |
| | QMAYRFNGIGVTQNVLYENQKLIANQFNSAIGKIQDSLSSTASALGKLQDVVNQNAQALN | |
| | TLVKQLSSNFGAISSVLNDILSRLDKVEAEVQIDRLITGRLQSLQTYVTQQLIRAAEIRA | |
| | SANLAATKMSECVLGQSKRVDFCGKGYHLMSFPQSAPHGVVFLHVTYVPAQEKNFTTAPA | |
| | ICHDGKAHFPREGVFVSNGTHWFVTQRNFYEPQIITTDNTFVSGNCDVVIGIVNNTVYDP | |
| | LQPELDSFKEELDKYFKNHTSPDVDLGDISGINASVVNIQKEIDRLNEVAKNLNESLIDL | |
| | QELGKYEQ | |
| 57 | VNLTTRTQLPPAYTNSFTRGVYYPDKVFRSSVLHSTQDLFLPFFSNVTWFHAIHVSGTNG | Prototypic |
| | TKRFDNPVLPFNDGVYFASTEKSNIIRGWIFGTTLDSKTQSLLIVNNATNVVIKVCEFQF | SARS-CoV-2 |
| | CNDPFLGVYYHKNNKSWMESEFRVYSSANNCTFEYVSQPFLMDLEGKQGNFKNLREFVFK | spike |
| | NIDGYFKIYSKHTPINLVRDLPQGFSALEPLVDLPIGINITRFQILLALHRSYLTPGDSS | protein NTD |
| | SGWTAGAAAYYVGYLQPRTFLLKYNENGTITDAVDCALDPLSETKCTLKS | without |
| | | signal |
| | | peptide, |
| | | 290 aa |
| 58 | PNITNLCPFGEVFNATRFASVYAWNRKRISNCVADYSVLYNSASFSTFKCYGVSPTKLND | Prototypic |
| | LCFTNVYADSFVIRGDEVRQIAPGQTGKIADYNYKLPDDFTGCVIAWNSNNLDSKVGGNY | SARS-CoV-2 |
| | NYLYRLFRKSNLKPFERDISTEIYQAGSTPCNGVEGFNCYFPLQSYGFQPTNGVGYQPYR | spike |
| | VVVLSFELLHAP | protein |
| | | RBD, 192 aa. |
| 59 | RRAR | Prototypic |
| | | SARS-CoV-2 |
| | | spike |
| | | protein |
| | | S1/S2 |
| 60 | GSAG | Prototypic |
| | | SARS-CoV-2 |
| | | spike |
| | | protein |
| | | S1/S2 |
| | | mutant |
| 61 | SFIEDLLFNKVTLADAGF | Prototypic |
| | | SARS-CoV-2 |
| | | spike |
| | | protein |
| | | fusion |
| | | peptide |
| | | (FP) |
| | | sequence |
| 62 | GIGVTQNVLYENQKLIANQFNSAIGKIQDSLSSTASALGKLQDVVNQNAQALNTLVKQLS | Prototypic |
| | SNFGAISSVLNDILSRLD | SARS-CoV-2 |
| | | spike |
| | | protein |
| | | heptad |
| | | repeat 1 |
| | | (HR1) |
| 63 | KVEAEVQIDRLITGRLQSLQTYVTQQLIRAAEIRASANLAATKMSECVLG | Prototypic |
| | | SARS-CoV-2 |
| | | spike |
| | | protein |
| | | central |
| | | helix (CH) |
| 64 | TTAPAICHDGKAHFPREGVFVSNGTHWFVTQRNFYEPQIITTDNTFVSGNCDVVIGIVNN | Prototypic |
| | TVYDPL | SARS-CoV-2 |
| | | spike |
| | | protein |
| | | connector |
| | | domain (CD) |
| 65 | EELDKYFKNHTSPDVDLGDISGINASVVNIQKEIDRLNEVAKNLNESLIDLQELGKYEQ | Prototypic |
| | | SARS-CoV-2 |
| | | spike |
| | | protein |
| | | heptad |
| | | repeat 2 |
| | | (HR2) |
| 66 | WPWYIWLGFIAGLIAIVMVTIML | Prototypic |
| | | SARS-CoV-2 |
| | | spike |
| | | protein |
| | | transmembrane |
| | | (TM) domain |
| 67 | ANVVRDRDLEVDTTLKSLSQQIENIRSPEGSRKNPARTCRDLKMCHSDWKSGEYWIDPNQ | Trimerization |
| | GCNLDAIKVFCNMETGETCVYPTQPSVAQKNWYISKNPKDKRHVWFGESMTDGFQFEYGG | peptide |
| | QGSDPADVAIQLTFLRLMSTEASQNITYHCKNSVAYMDQQTGNLKKALLLQGSNEIEIRA | (Type I), |
| | EGNSRFTYSVTVDGCTSHTGAWGKTVIEYKTTKTSRLPIIDVAPLDVGAPDQEFGFDVGP | QT version |
| | VCFL | |
| 68 | NGLPGPIGPPGPRGRTGDAGPVGPPGPPGPPGPPGPPSAGFDFSFLPQPPQEKAHDGGRY | Trimerization |
| | YRANDANVVRDRDLEVDTTLKSLSQQIENIRSPEGSRKNPARTCRDLKMCHSDWKSGEYW | peptide |
| | IDPNQGCNLDAIKVFCNMETGETCVYPTQPSVAQKNWYISKNPKDKRHVWFGESMTDGFQ | (Type 1), |
| | FEYGGQGSDPADVAIQLTFLRLMSTEASQNITYHCKNSVAYMDQQTGNLKKALLLQGSNE | with |
| | IEIRAEGNSRFTYSVTVDGCTSHTGAWGKTVIEYKTTKTSRLPIIDVAPLDVGAPDQEFG | glycine-X-Y |
| | FDVGPVCFL | repeats and |
| | | D→N |
| | | mutation at |
| | | BMP-1 site, |
| | | QT version |
| 69 | NGLPGPIGPPGPRGRTGDAGPVGPPGPPGPPGPPGPPSAGFDFSFLPQPPQEKAHDGGRY | Trimerization |
| | YRNDDANVVRDRDLEVDTTLKSLSQQIENIRSPEGSRKNPARTCRDLKMCHSDWKSGEYW | peptide |
| | IDPNQGCNLDAIKVFCNMETGETCVYPTQPSVAQKNWYISKNPKDKRHVWFGESMTDGFQ | (Type 1), |
| | FEYGGQGSDPADVAIQLTFLRLMSTEASQNITYHCKNSVAYMDQQTGNLKKALLLQGSNE | with |
| | IEIRAEGNSRFTYSVTVDGCTSHTGAWGKTVIEYKTTKTSRLPIIDVAPLDVGAPDQEFG | glycine-X-Y |
| | FDVGPVCFL | repeats and |
| | | A→N |
| | | mutation at |
| | | BMP-1 site, |
| | | QT version |
| 70 | RSNGLPGPIGPPGPRGRTGDAGPVGPPGPPGPPGPPGPPSAGFDFSFLPQPPQEKAHDGG | Trimerization |
| | RYYRANDANVVRDRDLEVDTTLKSLSQQIENIRSPEGSRKNPARTCRDLKMCHSDWKSGE | peptide |
| | YWIDPNQGCNLDAIKVFCNMETGETCVYPTQPSVAQKNWYISKNPKDKRHVWFGESMTDG | (Type 1), |
| | FQFEYGGQGSDPADVAIQLTFLRLMSTEASQNITYHCKNSVAYMDQQTGNLKKALLLQGS | with |
| | NEIEIRAEGNSRFTYSVTVDGCTSHTGAWGKTVIEYKTTKTSRLPIIDVAPLDVGAPDQE | glycine-X-Y |
| | FGFDVGPVCFL | repeats and |
| | | D→N |
| | | mutation at |
| | | BMP-1 site, |
| | | QT version |
| 71 | GSNGLPGPIGPPGPRGRTGDAGPVGPPGPPGPPGPPGPPSAGFDFSFLPQPPQEKAHDGG | Trimerization |
| | RYYRANDANVVRDRDLEVDTTLKSLSQQIENIRSPEGSRKNPARTCRDLKMCHSDWKSGE | peptide |
| | YWIDPNQGCNLDAIKVFCNMETGETCVYPTQPSVAQKNWYISKNPKDKRHVWFGESMIDG | (Type 1), |
| | FQFEYGGQGSDPADVAIQLTFLRLMSTEASQNITYHCKNSVAYMDQQTGNLKKALLLQGS | with |
| | NEIEIRAEGNSRFTYSVTVDGCTSHTGAWGKTVIEYKTTKTSRLPIIDVAPLDVGAPDQE | glycine-X-Y |
| | FGFDVGPVCFL | repeats and |
| | | D→N |
| | | mutation at |
| | | BMP-1 site, |
| | | QT version |
| 72 | ANVVRDRDLEVDTTLKSLSQQIENIRSPEGSRKNPARTCRDLKMCHSDWKSGEYWIDPNQ | Trimerization |
| | GCNLDAIKVFCNMETGETCVYPTQPSVAQKNWYISKNPKDKRHVWFGESMTDGFQFEYGG | peptide |
| | QGSDPADVAIQLTFLRLMSTEASQNITYHCKNSVAYMDQQTGNLKKALLLKGSNEIEIRA | (Type 1), |
| | EGNSRFTYSVTVDGCTSHTGAWGKTVIEYKTTKSSRLPIIDVAPLDVGAPDQEFGFDVGP | KS version |
| | VCFL | |
| 73 | NGLPGPIGPPGPRGRTGDAGPVGPPGPPGPPGPPGPPSAGFDFSFLPQPPQEKAHDGGRY | Trimerization |
| | YRANDANVVRDRDLEVDTTLKSLSQQIENIRSPEGSRKNPARTCRDLKMCHSDWKSGEYW | peptide |
| | IDPNQGCNLDAIKVFCNMETGETCVYPTQPSVAQKNWYISKNPKDKRHVWFGESMTDGFQ | (Type 1) |
| | FEYGGQGSDPADVAIQLTFLRLMSTEASQNITYHCKNSVAYMDQQTGNLKKALLLKGSNE | with |
| | IEIRAEGNSRFTYSVTVDGCTSHTGAWGKTVIEYKTTKSSRLPIIDVAPLDVGAPDQEFG | glycine-X-Y |
| | FDVGPVCFL | repeats and |
| | | D→N |
| | | mutation at |
| | | BMP-1 site, |
| | | KS version |
| 74 | NGLPGPIGPPGPRGRTGDAGPVGPPGPPGPPGPPGPPSAGFDFSFLPQPPQEKAHDGGRY | Trimerization |
| | YRNDDANVVRDRDLEVDTTLKSLSQQIENIRSPEGSRKNPARTCRDLKMCHSDWKSGEYW | peptide |
| | IDPNQGCNLDAIKVFCNMETGETCVYPTOPSVAQKNWYISKNPKDKRHVWFGESMIDGFQ | (Type 1) |
| | FEYGGQGSDPADVAIQLTFLRLMSTEASQNITYHCKNSVAYMDQQTGNLKKALLLKGSNE | with |
| | IEIRAEGNSRFTYSVTVDGCTSHTGAWGKTVIEYKTTKSSRLPIIDVAPLDVGAPDQEFG | glycine-X-Y |
| | FDVGPVCFL | repeats and |
| | | A→N |
| | | mutation at |
| | | BMP-1 site, |
| | | KS version |
| 75 | RSNGLPGPIGPPGPRGRTGDAGPVGPPGPPGPPGPPGPPSAGFDFSFLPQPPQEKAHDGG | Trimerization |
| | RYYRANDANVVRDRDLEVDTTLKSLSQQIENIRSPEGSRKNPARICRDLKMCHSDWKSGE | peptide |
| | YWIDPNQGCNLDAIKVFCNMETGETCVYPTQPSVAQKNWYISKNPKDKRHVWFGESMTDG | (Type 1) |
| | FQFEYGGQGSDPADVAIQLTFLRLMSTEASQNITYHCKNSVAYMDQQTGNLKKALLLKGS | glycine-X-Y |
| | NEIEIRAEGNSRFTYSVTVDGCTSHTGAWGKTVIEYKTTKSSRLPIIDVAPLDVGAPDQE | repeats and |
| | I'GEDVGE¹VCFL | D→N |
| | | mutation at |
| | | BMP-1 site, |
| | | KS version |
| 76 | GSNGLPGPIGPPGPRGRTGDAGPVGPPGPPGPPGPPGPPSAGFDFSFLPQPPQEKAHDGG | Trimerization |
| | RYYRANDANVVRDRDLEVDTTLKSLSQQIENIRSPEGSRKNPARTCRDLKMCHSDWKSGE | peptide |
| | YWIDPNQGCNLDAIKVFCNMETGETCVYPTQPSVAQKNWYISKNPKDKRHVWFGESMTDG | (Type 1) |
| | FQFEYGGQGSDPADVAIQLTFLRLMSTEASQNITYHCKNSVAYMDQQTGNLKKALLLKGS | with |
| | NEIEIRAEGNSRFTYSVTVDGCTSHTGAWGKTVIEYKTTKSSRLPIIDVAPLDVGAPDQE | glycine-X-Y |
| | FGFDVGPVCFL | repeats and |
| | | D→N |
| | | mutation at |
| | | BMP-1 site, |
| | | KS version |
| 77 | DEIMTSLKSVNGQIESLISPDGSRKNPARNCRDLKFCHPELKSGEYWVDPNQGCKLDAIK | Trimerization |
| | VFCNMETGETCISANPLNVPRKHWWTDSSAEKKHVWFGESMDGGFQFSYGNPELPEDVLD | peptide |
| | VQLAFLRLLSSRASQNITYHCKNSIAYMDQASGNVKKALKLMGSNEGEFKAEGNSKFTYT | (Type III) |
| | VLEDGCTKHTGEWSKTVFEYRTRKAVRLPIVDIAPYDIGGPDQEFGVDVGPVCF | |
| 78 | EPMDFKINTDEIMTSLKSVNGQIESLISPDGSRKNPARNCRDLKFCHPELKSGEYWVDPN | Trimerization |
| | QGCKLDAIKVFCNMETGETCISANPLNVPRKHWWTDSSAEKKHVWFGESMDGGFQFSYGN | peptide |
| | PELPEDVLDVQLAFLRLLSSRASQNITYHCKNSIAYMDQASGNVKKALKLMGSNEGEFKA | (Type III) |
| | EGNSKFTYTVLEDGCTKHTGEWSKTVFEYRTRKAVRLPIVDIAPYDIGGPDQEFGVDVGP | |
| 79 | SEPMDFKINTDEIMTSLKSVNGQIESLISPDGSRKNPARNCRDLKFCHPELKSGEYWVDP | Trimerization |
| | NQGCKLDAIKVFCNMETGETCISANPLNVPRKHWWTDSSAEKKHVWFGESMDGGFQFSYG | peptide |
| | NPELPEDVLDVQLAFLRLLSSRASQNITYHCKNSIAYMDQASGNVKKALKLMGSNEGEFK | (Type III) |
| | AEGNSKFTYTVLEDGCTKHTGEWSKTVFEYRTRKAVRLPIVDIAPYDIGGPDQEFGVDVG | |
| | PVCFL | |
| 80 | RSEPMDFKINTDEIMTSLKSVNGQIESLISPDGSRKNPARNCRDLKFCHPELKSGEYWVD | Trimerization |
| | PNQGCKLDAIKVFCNMETGETCISANPLNVPRKHWWTDSSAEKKHVWFGESMDGGFQFSY | peptide |
| | GNPELPEDVLDVQLAFLRLLSSRASQNITYHCKNSIAYMDQASGNVKKALKLMGSNEGEF | (Type III) |
| | KAEGNSKFTYTVLEDGCTKHTGEWSKTVFEYRTRKAVRLPIVDIAPYDIGGPDQEFGVDV | |
| | GPVCFL | |

1. A method for analyzing a sample, comprising:
contacting a sample with an antigen comprising a plurality of recombinant polypeptides, each recombinant polypeptide comprising a surface spike protein of a coronavirus linked to a C-terminal propeptide of collagen, wherein the C-terminal propeptides form inter-polypeptide disulfide bonds, and
wherein the sample contains or is suspected of containing an analyte capable of specific binding to the spike protein of the coronavirus, and a binding between the antigen and the analyte is detected.

2. The method of claim 1, wherein the analyte is an antibody, a receptor, a cell recognizing the antigen, and/or the sample is a body fluid, including but not limited to sera or plasma, which contain the analyte.

3. The method of claim 1, wherein the binding indicates the presence of the analyte in the sample, and/or an infection by the coronavirus in a subject from which the sample is derived.

4. The method of claim 1, wherein the method is a lateral flow method.

5. The method of any of claim 4, wherein the antigen is labeled with colloidal gold particles and dried within a conjugate pad on a test strip.

6. The method of claim 4, wherein a secondary antibody specific to the analyte is immobilized within a test zone of a chromatographic membrane on a test strip.

7. The method of claim 6, wherein the test strip further comprises a control zone wherein an antibody specific to a C-terminal propeptide of collagen is immobilized.

8. The method of claim 5, wherein the test strip further comprises a sample pad to which an analyte is loaded for analysis on one end of the test strip, and an absorbent pad on the opposite end which is in capillary communication with the sample pad.

9. The method of claim 4, wherein any successful retention of antigen-labeled colloidal gold particles at test zone, upon an analyte loading on to the sample pad as it migrates on the chromatographic membrane towards the absorbent pad via capillary force, indicates positive detection of an analyte, whereas retention of any antigen-labeled colloidal gold particles only at control zone indicates negative readout of the analyte.

10. The method of claim 1, wherein the analyte is an antibody against the surface antigen of a coronavirus.

11. The method of claim 1, wherein the analyte is a neutralizing antibody against the surface antigen of a coronavirus.

12. The method of any of claim 1, wherein the analyte is an IgG antibody or an IgM antibody.

13. (canceled)

14. The method of claim 1, wherein the analyte is a human antibody.

15. The method of claim 1, wherein the analyte is derived from a subject infected with the coronavirus.

16. The method of claim 1, wherein the analyte is serum from a subject infected with the coronavirus and has recovered.

17. The method of any of claim 1, wherein the analyte is derived from a subject immunized with a coronavirus vaccine.

18. The method of claim 1, wherein a receptor for the surface antigen of an coronavirus, optionally the receptor is a receptor-Fc, such as ACE2-Fc, is immobilized within a second test zone of a chromatographic membrane on a test strip.

19. The method of claim 17, wherein any reduction in retention of antigen-labeled colloidal gold particles at the second test zone upon loading an analyte, compared to vehicle control without analyte, indicates positive detection of neutralizing antibody or antibodies that is capable blocking the interaction between the receptor and the surface antigen of a coronavirus.

20. The method of any of claim 1, wherein the coronavirus is a Severe Acute Respiratory Syndrome (SARS)-coronavirus (SARS-CoV), a SARS-coronavirus 2 (SARS-CoV-2), a SARS-like coronavirus, a Middle East Respiratory Syndrome (MERS)-coronavirus (MERS-CoV), a MERS-like coronavirus, NL63-CoV, 229E-CoV, OC43-CoV, HKU1-CoV, WIV1-CoV, MHV, HKU9-CoV, PEDV-CoV, or SDCV.

21. The method of claim 1, wherein the antigen comprises a coronavirus spike (S) protein or a fragment or epitope thereof, wherein the epitope is optionally a linear epitope or a conformational epitope, and wherein the antigen comprises three recombinant antigen polypeptides linked by C-terminal propeptide of collagen.

22-52. (canceled)


SEQUENCE LISTING (80 sequences)

SEQ ID NO: 1 - protein, 1509 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 2 - protein, 1509 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 3 - protein, 1509 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 4 - protein, 1509 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 5 - protein, 836 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 6 - protein, 979 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 7 - protein, 837 aa, Artificial Sequence
  Synthetic construct
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 8 - protein, 827 aa, Artificial Sequence
  Synthetic construct
TMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 9 - protein, 707 aa, Artificial Sequence
  Synthetic construct
SFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 10 - protein, 1509 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 11 - protein, 1509 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 12 - protein, 1509 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 13 - protein, 1509 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 14 - protein, 1509 aa, Artificial Sequence
  Synthetic construct
QCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 15 - protein, 1509 aa, Artificial Sequence
  Synthetic construct
QCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 16 - protein, 1509 aa, Artificial Sequence
  Synthetic construct
QCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 17 - protein, 1509 aa, Artificial Sequence
  Synthetic construct
QCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 18 - protein, 1507 aa, Artificial Sequence
  Synthetic construct
QCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 19 - protein, 1507 aa, Artificial Sequence
  Synthetic construct
QCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 20 - protein, 1507 aa, Artificial Sequence
  Synthetic construct
QCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 21 - protein, 1507 aa, Artificial Sequence
  Synthetic construct
QCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 22 - protein, 1509 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 23 - protein, 1509 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 24 - protein, 1509 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 25 - protein, 1509 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 26 - protein, 1491 aa, Artificial Sequence
  Synthetic construct
SDLDRCTTFDDVQAPNYTQHTSSMRGVYYPDEIFRSDTLYLTQDLFLPFYSNVTGFHTINHTFDNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFLPAQDTWGTSAAAYFVGYLKPTTFMLKYDENGTITDAVDCSQNPLAELKCSVKSFEIDKGIYQTSNFRVVPSRDVVRFPNITNLCPFGEVFNATKFPSVYAWERKRISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKDFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQEEIDRLNEVAKNLNESLIDLQELGKYEQYIKRSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 27 - protein, 1195 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 28 - protein, 1195 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 29 - protein, 1195 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 30 - protein, 1195 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 31 - protein, 525 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKC

SEQ ID NO: 32 - protein, 668 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSP

SEQ ID NO: 33 - protein, 523 aa, Artificial Sequence
  Synthetic construct
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 34 - protein, 513 aa, Artificial Sequence
  Synthetic construct
TMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 35 - protein, 393 aa, Artificial Sequence
  Synthetic construct
SFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 36 - protein, 1195 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 37 - protein, 1195 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 38 - protein, 1195 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 39 - protein, 1195 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 40 - protein, 1195 aa, Artificial Sequence
  Synthetic construct
QCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 41 - protein, 1195 aa, Artificial Sequence
  Synthetic construct
QCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 42 - protein, 1195 aa, Artificial Sequence
  Synthetic construct
QCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 43 - protein, 1195 aa, Artificial Sequence
  Synthetic construct
QCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 44 - protein, 1193 aa, Artificial Sequence
  Synthetic construct
QCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 45 - protein, 1193 aa, Artificial Sequence
  Synthetic construct
QCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 46 - protein, 1193 aa, Artificial Sequence
  Synthetic construct
QCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 47 - protein, 1193 aa, Artificial Sequence
  Synthetic construct
QCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 48 - protein, 1195 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 49 - protein, 1195 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 50 - protein, 1195 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 51 - protein, 1195 aa, Artificial Sequence
  Synthetic construct
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 52 - protein, 1177 aa, Artificial Sequence
  Synthetic construct
SDLDRCTTFDDVQAPNYTQHTSSMRGVYYPDEIFRSDTLYLTQDLFLPFYSNVTGFHTINHTFDNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFLPAQDTWGTSAAAYFVGYLKPTTFMLKYDENGTITDAVDCSQNPLAELKCSVKSFEIDKGIYQTSNFRVVPSRDVVRFPNITNLCPFGEVFNATKFPSVYAWERKRISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKDFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQEEIDRLNEVAKNLNESLIDLQELGKYEQ

SEQ ID NO: 53 - protein, 13 aa, Artificial Sequence
  Synthetic construct
MFIFLLFLTLTSG

SEQ ID NO: 54 - protein, 13 aa, Artificial Sequence
  Synthetic construct
MFVFLVLLPLVSS

SEQ ID NO: 55 - protein, 1273 aa, Artificial Sequence
  Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 56 - protein, 1208 aa, Artificial Sequence
  Synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 57 - protein, 290 aa, Artificial Sequence
  Synthetic construct
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKS

SEQ ID NO: 58 - protein, 192 aa, Artificial Sequence
  Synthetic construct
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAP

SEQ ID NO: 59 - protein, 4 aa, Artificial Sequence
  Synthetic construct
RRAR

SEQ ID NO: 60 - protein, 4 aa, Artificial Sequence
  Synthetic construct
GSAG

SEQ ID NO: 61 - protein, 18 aa, Artificial Sequence
  Synthetic construct
SFIEDLLFNKVTLADAGF

SEQ ID NO: 62 - protein, 78 aa, Artificial Sequence
  Synthetic construct
GIGVTQNVLYENQKLIANQFNSAIGKIQDSLSSTASALGKLQDVVNQNAQALNTLVKQLSSNFGAISSVLNDILSRLD

SEQ ID NO: 63 - protein, 50 aa, Artificial Sequence
  Synthetic construct
KVEAEVQIDRLITGRLQSLQTYVTQQLIRAAEIRASANLAATKMSECVLG

SEQ ID NO: 64 - protein, 66 aa, Artificial Sequence
  Synthetic construct
TTAPAICHDGKAHFPREGVFVSNGTHWFVTQRNFYEPQIITTDNTFVSGNCDVVIGIVNNTVYDPL

SEQ ID NO: 65 - protein, 59 aa, Artificial Sequence
  Synthetic construct
EELDKYFKNHTSPDVDLGDISGINASVVNIQKEIDRLNEVAKNLNESLIDLQELGKYEQ

SEQ ID NO: 66 - protein, 23 aa, Artificial Sequence
  Synthetic construct
WPWYIWLGFIAGLIAIVMVTIML

SEQ ID NO: 67 - protein, 244 aa, Artificial Sequence
  Synthetic construct
ANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 68 - protein, 309 aa, Artificial Sequence
  Synthetic construct
NGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 69 - protein, 309 aa, Artificial Sequence
  Synthetic construct
NGLPGPIGPPGPRGRTGDAGPVGPPGPPGPPGPPGPPSAGFDFSFLPQPPQEKAHDGGRYYRNDDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 70 - protein, 311 aa, Artificial Sequence
  Synthetic construct
RSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 71 - protein, 311 aa, Artificial Sequence
  Synthetic construct
GSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLQGSNEIEIRAEGNSRFTYSVTVDGCTSHTGAWGKTVIEYKTTKTSRLPIIDVAPLDVGAPDQEFGFDVGPVCFL

SEQ ID NO: 72 - protein, 244 aa, Artificial Sequence
  Synthetic construct
ANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 73 - protein, 309 aa, Artificial Sequence
  Synthetic construct
NGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 74 - protein, 309 aa, Artificial Sequence
  Synthetic construct
NGLPGPIGPPGPRGRTGDAGPVGPPGPPGPPGPPGPPSAGFDFSFLPQPPQEKAHDGGRYYRNDDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 75 - protein, 311 aa, Artificial Sequence
  Synthetic construct
RSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 76 - protein, 311 aa, Artificial Sequence
  Synthetic construct
GSNGLPGPIGPPGPRGRTGDAGPVGPPGPPGPPGPPGPPSAGFDFSFLPQPPQEKAHDGGRYYRANDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKDKRHVWFGESMTDGFQFEYGGQGSDPADVAIQLTFLRLMSTEASQNITYHCKNSVAYMDQQTGNLKKALLLKGSNEIEIRAEGNSRFTYSVTVDGCTSHTGAWGKTVIEYKTTKSSRLPIIDVAPLDVGAPDQEFGFDVGPVCFL

SEQ ID NO: 77 - protein, 234 aa, Artificial Sequence
  Synthetic construct
DEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCF

SEQ ID NO: 78 - protein, 244 aa, Artificial Sequence
  Synthetic construct
EPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 79 - protein, 245 aa, Artificial Sequence
  Synthetic construct
SEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 80 - protein, 246 aa, Artificial Sequence
  Synthetic construct
RSEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVQLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL